SEQUENCE LISTING (83 sequences)

SEQ ID NO: 1 - protein, 153 aa, synthetic construct
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 2 - protein, 133 aa, synthetic construct [sequence omitted]

SEQ ID NO: 3 - protein, 133 aa, synthetic construct
APTSSSTKKTQLQLEHLLLDLQMILNGINNYKNPKLTRMLTFKFYMPKKATELKHLQCLEEELKPLEEALNLAQSKNFHLRPRDLISNINVIVLELKGSETTFMCEYADETATIVEFLNRWITFCQSIISTLT

SEQ ID NO: 4 - protein, 133 aa, synthetic construct [sequence omitted]

SEQ ID NO: 5 - protein, 133 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFCQSIISTLT

SEQ ID NO: 6 - protein, 133 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISRINVIVLELKGSETTFMCEYADETATIVEFLNRWITFCQSIISTLT

SEQ ID NO: 7 - protein, 133 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISSINVIVLELKGSETTFMCEYADETATIVEFLNRWITFCQSIISTLT

SEQ ID NO: 8 - protein, 133 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 9 - protein, 133 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFSQSIISTLT

SEQ ID NO: 10 - protein, 133 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISRINVIVLELKGSETTFMCEYADETATIVEFLNRWITFSQSIISTLT

SEQ ID NO: 11 - protein, 133 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISSINVIVLELKGSETTFMCEYADETATIVEFLNRWITFSQSIISTLT

SEQ ID NO: 12 - protein, 133 aa, synthetic construct
APTSSSTKKTQLQLEHLLLDLQMILNGINNYKNPKLTRMLTFKFYMPKKATELKHLQCLEEELKPLEEALNLAQSKNFHLRPRDLISDINVIVLELKGSETTFMCEYADETATIVEFLNRWITFSQSIISTLT

SEQ ID NO: 13 - protein, 133 aa, synthetic construct
APTSSSTKKTQLQLEHLLLDLQMILNGINNYKNPKLTRMLTFKFYMPKKATELKHLQCLEEELKPLEEALNLAQSKNFHLRPRDLISRINVIVLELKGSETTFMCEYADETATIVEFLNRWITFSQSIISTLT

SEQ ID NO: 14 - protein, 133 aa, synthetic construct
APTSSSTKKTQLQLEHLLLDLQMILNGINNYKNPKLTRMLTFKFYMPKKATELKHLQCLEEELKPLEEALNLAQSKNFHLRPRDLISSINVIVLELKGSETTFMCEYADETATIVEFLNRWITFSQSIISTLT

SEQ ID NO: 15 - protein, 133 aa, synthetic construct
APTSSSTKKTQLQLEHLLLDLQMILNGINNYKNPKLTRMLTFKFYMPKKATELKHLQCLEEELKPLEEVLNLAPSKNFHLRPRDLISDINVIVLELKGSETTFMCEYADETATIVEFLNRWITFSQSIISTLT

SEQ ID NO: 16 - protein, 133 aa, synthetic construct; identical to SEQ ID NO: 15

SEQ ID NO: 17 - protein, 133 aa, synthetic construct; identical to SEQ ID NO: 15

SEQ ID NO: 18 - protein, 133 aa, synthetic construct
APTSSSTKKTQLQLEHLLLDLQMILNGINNYKNPKLTRMLTFKFYMPKKATELKHLQCLEEELKPLEEALNLAPSKNFHLRPRDLISDINVIVLELKGSETTFMCEYADETATIVEFLNRWITFSQSIISTLT

SEQ ID NO: 19 - protein, 133 aa, synthetic construct
APTSSSTKKTQLQLEHLLLDLQMILNGINNYKNPKLTRMLTFKFYMPKKATELKHLQCLEEELKPLEEALNLAPSKNFHLRPRDLISRINVIVLELKGSETTFMCEYADETATIVEFLNRWITFSQSIISTLT

SEQ ID NO: 20 - protein, 133 aa, synthetic construct
APTSSSTKKTQLQLEHLLLDLQMILNGINNYKNPKLTRMLTFKFYMPKKATELKHLQCLEEELKPLEEALNLAPSKNFHLRPRDLISSINVIVLELKGSETTFMCEYADETATIVEFLNRWITFSQSIISTLT

SEQ ID NO: 21 - protein, 159 aa, synthetic construct
SITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 22 - protein, 159 aa, synthetic construct
SITGISPITEYLASLSTYNDQSITFALEDESYEIYVEDLKKDEKKDKVLLSYYESQHPSSESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 23 - protein, 159 aa, synthetic construct
SITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNFVSFECKTDPGVFIGVKDNHLALIKVDSSENLCTENILFKLSET

SEQ ID NO: 24 - protein, 159 aa, synthetic construct
SITGISPITEYLASLSTYNDQSITFALEDESYEIYVEDLKKDEKKDKVLLSYYESQHPSNESGDGVDGKMLMVTLSPTKDFWLHANNKEHSVELHKGEKPLPDQAFFVLHNMHSNCVSFECKTDPGVFIGVKDNHLALIKVDSSENLCTENILFKLSET

SEQ ID NO: 25 - protein, 159 aa, synthetic construct
SITGISPITEYLASLSTYNDQSITFALEDESYEIYVEDLKKDEKKDKVLLSYYESQHPSSESGDGVDGKMLMVTLSPTKDFWLHANNKEHSVELHKCEKPLPDQAFFVLHNMHSNFVSFECKTDPGVFIGVKDNHLALIKVDSSENLCTENILFKLSET

SEQ ID NO: 26 - protein, 159 aa, synthetic construct
SITGISPITEYLASLSTYNDQSITFALEDESYEIYVEDLKKDEKKDKVLLSYYESQHPSSESGDGVDGKMLMVTLSPTKDFWLHANNKEHSVELHKGEKPLPDQAFFVLHNMHSNCVSFECKTDPGVFIGVKDNHLALIKVDSSENLCTENILFKLSET

SEQ ID NO: 27 - protein, 159 aa, synthetic construct
SITGISPITEYLASLSTYNDQSITFALEDESYEIYVEDLKKDEKKDKVLLSYYESQHPSNESGDGVDGKMLMVTLSPTKDFWLHANNKEHSVELHKGEKPLPDQAFFVLHNMHSNFVSFECKTDPGVFIGVKDNHLALIKVDSSENLCTENILFKLSET

SEQ ID NO: 28 - protein, 159 aa, synthetic construct
SITGISPITEYLASLSTYNDQSITFALEDESYEIYVEDLKKDEKKDKVLLSYYESQHPSSESGDGVDGKMLMVTLSPTKDFWLHANNKEHSVELHKGEKPLPDQAFFVLHNMHSNFVSFECKTDPGVFIGVKDNHLALIKVDSSENLCTENILFKLSET

SEQ ID NO: 29 - protein, 5 aa, synthetic construct
GGGGS

SEQ ID NO: 30 - protein, 15 aa, synthetic construct
GGGGSGGGGSGGGGS

SEQ ID NO: 31 - protein, 7 aa, synthetic construct
GGSGGGS

SEQ ID NO: 32 - protein, 5 aa, synthetic construct
EAAAK

SEQ ID NO: 33 - protein, 8 aa, synthetic construct
GGGGGGGG

SEQ ID NO: 34 - protein, 10 aa, synthetic construct
GSGSGSGSGS

SEQ ID NO: 35 - protein, 20 aa, synthetic construct
GSGSGSGSGSGSGSGSGSGS

SEQ ID NO: 36 - protein, 9 aa, synthetic construct
GGSGGSGGS

SEQ ID NO: 37 - protein, 5 aa, synthetic construct
GSGGS

SEQ ID NO: 38 - protein, 5 aa, synthetic construct
GSSGS

SEQ ID NO: 39 - protein, 4 aa, synthetic construct
GSAT

SEQ ID NO: 40 - protein, 10 aa, synthetic construct
SSSSGSSSSG

SEQ ID NO: 41 - protein, 22 aa, synthetic construct
LEGSGQGPGSGQGSGSPGSGQG

SEQ ID NO: 42 - protein, 10 aa, synthetic construct
GGGGSEAAAK

SEQ ID NO: 43 - protein, 20 aa, synthetic construct
GGGGSEAAAKGGGGSEAAAK

SEQ ID NO: 44 - protein, 14 aa, synthetic construct
EGKSSGSGSESKST

SEQ ID NO: 45 - protein, 16 aa, synthetic construct
GGGGSLVPRGSGGGGS

SEQ ID NO: 46 - protein, 18 aa, synthetic construct
KESGSVSSEQLAQFRSLD

SEQ ID NO: 47 - protein, 18 aa, synthetic construct
GGGSEGGGSEGGGSEGGG

SEQ ID NO: 48 - protein, 5 aa, synthetic construct
PAPAP

SEQ ID NO: 49 - protein, 12 aa, synthetic construct
AEAAAKEAAAKA

SEQ ID NO: 50 - protein, 46 aa, synthetic construct
AEAAAKEAAAKEAAAKEAAAKALEAEAAAKEAAAKEAAAKEAAAKA

SEQ ID NO: 51 - protein, 24 aa, synthetic construct
KAPPPSLPSPSRLPGPSDTPILPQ

SEQ ID NO: 52 - protein, 19 aa, synthetic construct
MGWSCIILFLVATATGVHS

SEQ ID NO: 53 - protein, 8 aa, synthetic construct
DYKDDDDK

SEQ ID NO: 54 - protein, 326 aa, synthetic construct
MGWSCIILFLVATATGVHSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 55 - protein, 326 aa, synthetic construct
MGWSCIILFLVATATGVHSAPTSSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFCQSIISTLTGGGGSGGGGSGGGGSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 56 - protein, 326 aa, synthetic construct
MGWSCIILFLVATATGVHSAPTSSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFCQSIISTLTGGGGSGGGGSGGGGSSITGISPITEYLASLSTYNDQSITFALEDESYEIYVEDLKKDEKKDKVLLSYYESQHPSSESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 57 - protein, 326 aa, synthetic construct
MGWSCIILFLVATATGVHSAPTSSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFCQSIISTLTGGGGSGGGGSGGGGSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNFVSFECKTDPGVFIGVKDNHLALIKVDSSENLCTENILFKLSET

SEQ ID NO: 58 - protein, 326 aa, synthetic construct
MGWSCIILFLVATATGVHSAPTSSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFCQSIISTLTGGGGSGGGGSGGGGSSITGISPITEYLASLSTYNDQSITFALEDESYEIYVEDLKKDEKKDKVLLSYYESQHPSNESGDGVDGKMLMVTLSPTKDFWLHANNKEHSVELHKGEKPLPDQAFFVLHNMHSNCVSFECKTDPGVFIGVKDNHLALIKVDSSENLCTENILFKLSET

SEQ ID NO: 59 - protein, 326 aa, synthetic construct
MGWSCIILFLVATATGVHSAPTSSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFCQSIISTLTGGGGSGGGGSGGGGSSITGISPITEYLASLSTYNDQSITFALEDESYEIYVEDLKKDEKKDKVLLSYYESQHPSSESGDGVDGKMLMVTLSPTKDFWLHANNKEHSVELHKGEKPLPDQAFFVLHNMHSNFVSFECKTDPGVFIGVKDNHLALIKVDSSENLCTENILFKLSET

SEQ ID NO: 60 - protein, 326 aa, synthetic construct
MGWSCIILFLVATATGVHSAPTSSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFCQSIISTLTGGGGSGGGGSGGGGSSITGISPITEYLASLSTYNDQSITFALEDESYEIYVEDLKKDEKKDKVLLSYYESQHPSNESGDGVDGKMLMVTLSPTKDFWLHANNKEHSVELHKGEKPLPDQAFFVLHNMHSNFVSFECKTDPGVFIGVKDNHLALIKVDSSENLCTENILFKLSET

SEQ ID NO: 61 - protein, 326 aa, synthetic construct
MGWSCIILFLVATATGVHSAPTSSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFSQSIISTLTGGGGSGGGGSGGGGSSITGISPITEYLASLSTYNDQSITFALEDESYEIYVEDLKKDEKKDKVLLSYYESQHPSNESGDGVDGKMLMVTLSPTKDFWLHANNKEHSVELHKGEKPLPDQAFFVLHNMHSNFVSFECKTDPGVFIGVKDNHLALIKVDSSENLCTENILFKLSET

SEQ ID NO: 62 - protein, 326 aa, synthetic construct
MGWSCIILFLVATATGVHSAPTSSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFSQSIISTLTGGGGSGGGGSGGGGSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNFVSFECKTDPGVFIGVKDNHLALIKVDSSENLCTENILFKLSET

SEQ ID NO: 63 - protein, 567 aa, synthetic construct
MGWSCIILFLVATATGVHSAPTSSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFSQSIISTLTGGGGSGGGGSGGGGSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNFVSFECKTDPGVFIGVKDNHLALIKVDSSENLCTENILFKLSETGGGGSGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 64 - protein, 326 aa, synthetic construct
MGWSCIILFLVATATGVHSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNFVSFECKTDPGVFIGVKDNHLALIKVDSSENLCTENILFKLSET

SEQ ID NO: 65 - protein, 567 aa, synthetic construct
MGWSCIILFLVATATGVHSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTGGGGSGGGGSGGGGSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNFVSFECKTDPGVFIGVKDNHLALIKVDSSENLCTENILFKLSETGGGGSGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 66 - protein, 568 aa, synthetic construct
MGWSCIILFLVATATGVHSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTGGGGSGGGGSGGGGSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNFVSFECKTDPGVFIGVKDNHLALIKVDSSENLCTENILFKLSET

SEQ ID NO: 67 - protein, 568 aa, synthetic construct
MGWSCIILFLVATATGVHSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFSQSIISTLTGGGGSGGGGSGGGGSSITGISPITEYLASLSTYNDQSITFALEDESYEIYVEDLKKDEKKDKVLLSYYESQHPSNESGDGVDGKMLMVTLSPTKDFWLHANNKEHSVELHKGEKPLPDQAFFVLHNMHSNFVSFECKTDPGVFIGVKDNHLALIKVDSSENLCTENILFKLSET

SEQ ID NO: 68 - protein, 326 aa, synthetic construct
MGWSCIILFLVATATGVHSAPTSSSTKKTQLQLEHLLLDLQMILNGINNYKNPKLTRMLTFKFYMPKKATELKHLQCLEEELKPLEEALNLAPSKNFHLRPRDLISDINVIVLELKGSETTFMCEYADETATIVEFLNRWITFSQSIISTLTGGGGSGGGGSGGGGSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNFVSFECKTDPGVFIGVKDNHLALIKVDSSENLCTENILFKLSET

SEQ ID NO: 69 - protein, 326 aa, synthetic construct
MGWSCIILFLVATATGVHSAPTSSSTKKTQLQLEHLLLDLQMILNGINNYKNPKLTRMLTFKFYMPKKATELKHLQCLEEELKPLEEALNLAPSKNFHLRPRDLISDINVIVLELKGSETTFMCEYADETATIVEFLNRWITFSQSIISTLTGGGGSGGGGSGGGGSSITGISPITEYLASLSTYNDQSITFALEDESYEIYVEDLKKDEKKDKVLLSYYESQHPSNESGDGVDGKMLMVTLSPTKDFWLHANNKEHSVELHKGEKPLPDQAFFVLHNMHSNFVSFECKTDPGVFIGVKDNHLALIKVDSSENLCTENILFKLSET

SEQ ID NO: 70 - protein, 326 aa, synthetic construct
MGWSCIILFLVATATGVHSAPTSSSTKKTQLQLEHLLLDLQMILNGINNYKNPKLTRMLTFKFYMPKKATELKHLQCLEEELKPLEEALNLAPSKNFHLRPRDLISDINVIVLELKGSETTFMCEYADETATIVEFLNRWITFCQSIISTLTGGGGSGGGGSGGGGSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNFVSFECKTDPGVFIGVKDNHLALIKVDSSENLCTENILFKLSET

SEQ ID NO: 71 - protein, 334 aa, synthetic construct
MGWSCIILFLVATATGVHSDYKDDDDKAPTSSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFSQSIISTLTGGGGSGGGGSGGGGSSITGISPITEYLASLSTYNDQSITFALEDESYEIYVEDLKKDEKKDKVLLSYYESQHPSNESGDGVDGKMLMVTLSPTKDFWLHANNKEHSVELHKGEKPLPDQAFFVLHNMHSNFVSFECKTDPGVFIGVKDNHLALIKVDSSENLCTENILFKLSET

SEQ ID NO: 72 - protein, 334 aa, synthetic construct
MGWSCIILFLVATATGVHSDYKDDDDKAPTSSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFSQSIISTLTGGGGSGGGGSGGGGSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNFVSFECKTDPGVFIGVKDNHLALIKVDSSENLCTENILFKLSET

SEQ ID NO: 73 - protein, 334 aa, synthetic construct
MGWSCIILFLVATATGVHSDYKDDDDKAPTSSSTKKTQLQLEHLLLDLQMILNGINNYKNPKLTRMLTFKFYMPKKATELKHLQCLEEELKPLEEALNLAPSKNFHLRPRDLISDINVIVLELKGSETTFMCEYADETATIVEFLNRWITFSQSIISTLTGGGGSGGGGSGGGGSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNFVSFECKTDPGVFIGVKDNHLALIKVDSSENLCTENILFKLSET

SEQ ID NO: 74 - protein, 334 aa, synthetic construct
MGWSCIILFLVATATGVHSDYKDDDDKAPTSSSTKKTQLQLEHLLLDLQMILNGINNYKNPKLTRMLTFKFYMPKKATELKHLQCLEEELKPLEEALNLAPSKNFHLRPRDLISDINVIVLELKGSETTFMCEYADETATIVEFLNRWITFSQSIISTLTGGGGSGGGGSGGGGSSITGISPITEYLASLSTYNDQSITFALEDESYEIYVEDLKKDEKKDKVLLSYYESQHPSNESGDGVDGKMLMVTLSPTKDFWLHANNKEHSVELHKGEKPLPDQAFFVLHNMHSNFVSFECKTDPGVFIGVKDNHLALIKVDSSENLCTENILFKLSET

SEQ ID NO: 75 - protein, 334 aa, synthetic construct
MGWSCIILFLVATATGVHSDYKDDDDKAPTSSSTKKTQLQLEHLLLDLQMILNGINNYKNPKLTRMLTFKFYMPKKATELKHLQCLEEELKPLEEALNLAPSKNFHLRPRDLISDINVIVLELKGSETTFMCEYADETATIVEFLNRWITFCQSIISTLTGGGGSGGGGSGGGGSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNFVSFECKTDPGVFIGVKDNHLALIKVDSSENLCTENILFKLSET

SEQ ID NO: 76 - protein, 186 aa, synthetic construct
MGWSCIILFLVATATGVHSDYKDDDDKSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 77 - protein, 186 aa, synthetic construct
MGWSCIILFLVATATGVHSDYKDDDDKSITGISPITEYLASLSTYNDQSITFALEDESYEIYVEDLKKDEKKDKVLLSYYESQHPSNESGDGVDGKMLMVTLSPTKDFWLHANNKEHSVELHKGEKPLPDQAFFVLHNMHSNFVSFECKTDPGVFIGVKDNHLALIKVDSSENLCTENILFKLSET

SEQ ID NO: 78 - protein, 159 aa, synthetic construct
SITGISPITEYLASLSTYNDQSITFALEDESYEIYVEDLKKDEKKDKVLLSYYESQHPSSESGDGVDGKMLMVTLSPTKDFWLHANNKEHSVELHKGEKPLPDQAFFVLHNMHSNFVSFECKTDPGVFIGVKDNHLALIKVDSSENLGTENILFKLSET

SEQ ID NO: 79 - protein, 159 aa, synthetic construct
SITGISPITEYLASLSTYNDQSITFALEDESYEIYVEDLKKDEKKDKVLLSYYESQHPSSESGDGVDGKMLMVTLSPTKDFWLHANNKEHSVELHKGEKPLPDQAFFVLHNMHSNFVSFESKTDPGVFIGVKDNHLALIKVDSSENLGTENILFKLSET

SEQ ID NO: 80 - protein, 326 aa, synthetic construct
MGWSCIILFLVATATGVHSAPTSSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFSQSIISTLTGGGGSGGGGSGGGGSSITGISPITEYLASLSTYNDQSITFALEDESYEIYVEDLKKDEKKDKVLLSYYESQHPSNESGDGVDGKMLMVTLSPTKDFWLHANNKEHSVELHKGEKPLPDQAFFVLHNMHSNFVSFECKTDPGVFIGVKDNHLALIKVDSSENLGTENILFKLSET

SEQ ID NO: 81 - protein, 326 aa, synthetic construct
MGWSCIILFLVATATGVHSAPTSSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFSQSIISTLTGGGGSGGGGSGGGGSSITGISPITEYLASLSTYNDQSITFALEDESYEIYVEDLKKDEKKDKVLLSYYESQHPSNESGDGVDGKMLMVTLSPTKDFWLHANNKEHSVELHKGEKPLPDQAFFVLHNMHSNFVSFESKTDPGVFIGVKDNHLALIKVDSSENLGTENILFKLSET

SEQ ID NO: 82 - protein, 186 aa, synthetic construct
MGWSCIILFLVATATGVHSDYKDDDDKSITGISPITEYLASLSTYNDQSITFALEDESYEIYVEDLKKDEKKDKVLLSYYESQHPSNESGDGVDGKMLMVTLSPTKDFWLHANNKEHSVELHKGEKPLPDQAFFVLHNMHSNFVSFECKTDPGVFIGVKDNHLALIKVDSSENLGTENILFKLSET

SEQ ID NO: 83 - protein, 186 aa, synthetic construct
MGWSCIILFLVATATGVHSDYKDDDDKSITGISPITEYLASLSTYNDQSITFALEDESYEIYVEDLKKDEKKDKVLLSYYESQHPSNESGDGVDGKMLMVTLSPTKDFWLHANNKEHSVELHKGEKPLPDQAFFVLHNMHSNFVSFESKTDPGVFIGVKDNHLALIKVDSSENLGTENILFKLSET

(19) United States
(12) Patent Application Publication
(10) Pub. No.: US 2026/0258109 A1
(43) Pub. Date: Sep. 3, 2026

(54) BIFUNCTIONAL IL-2 AND IL-33 PROTEINS FOR TREATING AUTOIMMUNE DISEASE

(71) Applicants: State Bio, Inc., Charlottesville, VA (US); UNIVERSITY OF VIRGINIA PATENT FOUNDATION, Charlottesville, VA (US)

(72) Inventors: Michelle SOLTERO HIGGIN, Charlottesville, VA (US); Rahul SHARMA, Charlottesville, VA (US); Thomas Charles BOONE, Charlottesville, VA (US); John P. WELSH, Charlottesville, VA (US)

(21) Appl. No.: 19/676,467

(22) Filed: May 13, 2026

Related U.S. Application Data
(63) Continuation of application No. PCT/US2024/055931, filed on Nov. 14, 2024.
(60) Provisional application No. 63/599,170, filed on Nov. 15, 2023.

(51) Int. Cl. C07K 14/55; A61K 38/00; A61P 37/06

(52) U.S. Cl. CPC C07K 14/55; A61P 37/06; A61K 38/00; C07K 2319/02

(57) ABSTRACT

The present invention provides, among other things, compositions and methods for prophylaxis and treatment of autoimmune disease. The present invention provides, in part, bifunctional proteins comprising interleukin-2 (IL-2) and interleukin-33 (IL-33) variants having increased manufacturability and activity, which synergistically expand or stimulate tissue targeting or reparative regulatory T cells. The present invention provides compositions and methods for proliferation and activity of ST2+ regulatory T cells.

### CROSS-REFERENCE TO PRIORITY APPLICATIONS

The present application is a Continuation Application of International Application No. PCT/US2024/055931, filed on Nov. 14, 2024, which claims priority to U.S. Provisional Application No. 63/599,170 filed on Nov. 15, 2023; the contents of each of which are incorporated herein by reference for all purposes.

### INCORPORATION BY REFERENCE OF SEQUENCE LISTING

The present application is being filed along with a Sequence Listing submitted electronically in XML format. The Sequence Listing file “SLB-006US1_SL.xml” is created on May 11, 2026, which is 86,236 bytes in size; the contents of which are incorporated herein for all purposes.

### BACKGROUND OF THE INVENTION

Autoimmune disease typically occurs due to a failure of the immune system to distinguish between self and non-self tissue, thereby attacking and destroying cells and tissues of the body. Regulatory T cells (Treg) play a role in suppressing pathological activation of the immune system and prevent autoimmune disease. Treg cell numbers or cell function are reduced in several autoimmune and inflammatory diseases.

CD4+CD25+ Treg cells suppress the activity of other immune cells, and maintain tolerance to self-antigens, modulating responses to foreign antigens and regulation of the immune system. T cells that express CD25 include FOXP3+CD4+ regulatory T cells (Treg cells), which are essential for suppressing autoimmune inflammation. On the other hand, FOXP3− T effector cells activated to express CD25, may be either CD4+ or CD8+ and contribute to inflammation, autoimmunity, organ graft rejection, or graft-versus-host disease. IL-2-stimulated STAT5 signaling is believed to be important for normal T-reg cell growth and survival and for high FOXP3 expression.

Treg cells maintain inflammatory responses in various disease states. A subset of suppressive CD4+ Foxp3+ Treg cells, ST2+ Treg cells binds Interleukin-33 (IL-33) through an IL-33 receptor (IL-33R also known as ST2) and are enriched in inflammatory diseases and tissues. ST2+ Treg cells are tissue targeting, more suppressive and reparative than systemic Treg cells. IL-33 signals through NFkB and MAPK pathways and independently stimulates Treg cells.

One approach for treating autoimmune diseases is the transplantation of autologous, ex vivo expanded Treg cells. Although successful in animal models and early stage human clinical trials, this approach is challenging since it is technically complex and invasive because it requires personalized treatment with the patient's own T-cells.

Recombinant IL-2, Proleukin (Prometheus Laboratories, San Diego) is approved for treatment of metastatic melanoma and metastatic renal cancer, but it is associated with severe side-effects due to high toxicity. Clinical treatment with low dose IL-2 has been used in chronic GVHD and HCV-associated autoimmune vasculitis and demonstrated increased Treg levels. However, therapeutic administration of recombinant IL-2 results in undesirable toxicity and short half life, for example, due to the non-specific activation of NK cells. IL-2 alone, even with extended half-life and altered receptor binding specificity is not sufficient for optimum expansion and activation of Tregs for tissue targeting. Therefore, there is a need for a therapeutic agent for preventing and/or treating autoimmune disease that is safe and tolerable for treating humans.

### SUMMARY OF THE INVENTION

The present invention provides, among other things, bifunctional proteins comprising an interleukin-2 (IL-2) polypeptide and an interleukin-33 (IL-33) variant having improved activity and/or manufacturability, that preferentially activate proliferation and/or activity of tissue resident, suppressive or reparative subset of regulatory T cells (Treg), also known as ST2+ Treg cells. Provided herein are compositions and methods for proliferation and activity of these suppressive reparative Treg cells, which are mobilized to local tissues, and facilitate homoeostasis in the local tissue microenvironment resulting in a safe, potent and durable disease response.

The IL-33 variants of the present disclosure are engineered for reduced oxidation, improved activity, better selectivity and/or manufacturability relative to wild-type IL-33. The IL-2 polypeptides of the present disclosure function to enhance selectivity of the ratio of Treg to T effector cell activity for treating autoimmune or inflammatory disease.

The present invention is based, in part, on the surprising discovery that bifunctional proteins comprising IL-2 polypeptides associated with exemplary IL-33 variants synergistically expand or stimulate ST2+ Treg cells or reparative Treg cells. IL-2 systemically upregulates the IL-33 receptor (also known as ST2) on Treg cells while IL-33, which is released in tissues, also expands Treg cells independently of IL-2. The inventors of the present application found that the bifunctional protein showed a complementary and synergistic effect in expanding ST2+ tissue targeting Treg cells that was surprisingly and unexpectedly more efficacious than either cytokine alone.

Among other things, the bifunctional protein results in colocalization and coexpression of IL-2 and IL-33 variant in the same tissue. The bifunctional protein is active on both IL-2Ra (CD25) receptor and IL-33 (ST2) receptor, either simultaneously or sequentially, increasing local concentration and avidity and effectively driving downstream signaling activity through two pathways—IL-2 through the Jak-STAT pathway and IL-33 through the NFκB/MAPK pathway, thereby, greatly increasing activity and efficacy of the bifunctional fusion protein. The bifunctional fusion protein preferentially proliferates ST2+ Treg cells both systemically, in peripheral blood, and importantly, at the local site of tissue injury or inflammation producing a tolerogenic tissue microenvironment. The bifunctional protein of the present disclosure expands total Tregs and increases the proportion of ST2+ and CD25 bright Treg subsets that localize to tissue.

Provided herein are improved compositions and methods having increased efficacy, potency, tissue targeting and avidity for prophylaxis and treatment of autoimmune disease, including, as non-limiting examples, Type 1 diabetes (T1D), Systemic Lupus Erythematosus (SLE), Acute Kidney Injury (AKI) and Graft vs. Host Disease (GVHD).

In some aspects, provided herein is a bifunctional protein comprising: an IL-2 polypeptide, and an IL-33 variant that has improved activity and/or manufacturability as compared to human WT IL-33, wherein the IL-2 polypeptide is associated with the IL-33 variant, and wherein the bifunctional protein preferentially expands ST2+ Treg cells.

In some embodiments of the bifunctional protein, the IL-2 polypeptide is linked to the IL-33 variant via a peptide linker or a chemical linker. In some embodiments, the IL-2 is at the N-terminus. In some embodiments, the IL-2 is at the C-terminus. In some embodiments, the IL-33 is at the N-terminus. In some embodiments, the IL-33 is at the C-terminus.

In some embodiments, the linker is a peptide linker comprising 3-30 amino acids.

In some embodiments, the bifunctional protein comprises an IL-2 polypeptide and an IL-33 polypeptide, wherein the IL-2 polypeptide and the IL-33 polypeptide are associated with each other at a distance of between 1 nm to 10 nm.

In some aspects, provided herein is a bifunctional protein comprising an IL-2 polypeptide and an IL-33 polypeptide, wherein the IL-2 polypeptide and the IL-33 polypeptide are associated with each other at a distance of between 10 Å to 100 Å.

In some embodiments, the distance is about 1.5 nm to 10 nm, 3.5 nm to 10 nm, 3.5 nm to 7 nm, 5 nm to 10 nm, or 5 nm to 7 nm.

In some embodiments, the distance is about 15 Å to 100 Å, 35 Å to 100 Å, 35 Å to 70 Å, 50 Å to 100 Å, or 50 Å to 70 Å.

In some embodiments, the IL-2 polypeptide and the IL-33 polypeptide are linked by a linker.

In some embodiments, the linker is a peptide linker or a chemical linker.

In some aspects, provided herein is a bifunctional protein comprising from the N-terminus to the C-terminus, an IL-2 polypeptide, a linker, and an IL-33 variant that has improved activity and/or manufacturability as compared to human WT IL-33, wherein the linker comprises 3-30 amino acids.

In some embodiments, the IL-33 variant or polypeptide is a truncated IL-33 and at least one amino acid substitution at positions selected from N60, C97, C116, C121, and C148 according to the sequence of SEQ ID NO: 21.

In some embodiments, the IL-33 variant or polypeptide comprises two, three, four or five amino acid substitutions selected from positions of N60, C97, C116, C121 and C148.

In some embodiments, the IL-33 variant or polypeptide comprises an amino acid substitution at position N60, wherein the substitution is N60S, N60T, N60D N60E or N60A.

In some embodiments, the IL-33 variant or polypeptide comprises an amino acid substitution at position C97, wherein the substitution is C97G, C97A or C97V.

In some embodiments, the IL-33 variant or polypeptide comprises an amino acid substitution at position C116, wherein the substitution is C116F, C116Y or C116A.

In some embodiments, the IL-33 variant or polypeptide comprises an amino acid substitution at position C121, wherein the substitution is C121S or C121A.

In some embodiments, the IL-33 variant or polypeptide comprises an amino acid substitution at position C148, wherein the substitution is C148G, C148S, C148Y, C148N or C148A.

In some embodiments, the IL-33 variant or polypeptide comprises the amino acid substitutions of N60S, C97G and C116F.

In some embodiments, the IL-33 variant or polypeptide further comprises an amino acid substitution C148G.

In some embodiments, the IL-33 variant or polypeptide further comprises an amino acid substitution C121S.

In some embodiments of the bifunctional protein, the IL-2 polypeptide comprises one or more amino acid substitutions selected from the group consisting of T3N, T3A, L12G, L12K, L12Q, L12S, Q13G, E15A, E15G, E15S, H16A, H16D, H16E, H16G, H16K, H16M, H16N, H16R, H16S, H16T, H16V, H16Y, L19A, L19D, L19E, L19G, L19N, L19R, L19S, L19T, L19V, D20A, D20E, D20H, D201, D20Y, D20F, D20G, D20T, D20W, M23R, V69A, Q47P, R81A, R81G, R81S, R81T, D84A, D84E, D84G, D84K, D841, D84M, D84Q D84R, D84S, D84T, S87R, N88A, N88D, N88R, N88E, N88I, N88F, N88G, N88M, N88R, N88S, N88V, N88W, V91D, V91E, V91K, V91G, V91N, V91R, V91S, 192K, 192R, E95G, C125A, C125S, Q1261, Q126L, and Q126F.

In some embodiments, the IL-2 polypeptide comprises one or more amino acid substitutions selected from V69A, Q47P, N88D, N88R and C125S.

In some embodiments, the IL-2 polypeptide comprises amino acid substitutions of N88D and C125S.

In some embodiments, the bifunctional protein is capable of binding to CD25 and ST2 on the surface of a single cell. In some embodiments, the bifunctional protein is capable of binding to CD25 and ST2 on the surface of a single cell at the same time. In some embodiments, the bifunctional protein is capable of binding to CD25 and ST2 on the surface of a single cell at different times.

In some embodiments, the bifunctional protein is capable of binding to CD25 and ST2 on the surface of different cells.

In some embodiments, the bifunctional protein expands circulating FoxP3+ Treg cells, CD25^{bright} Treg cells, tissue targeting ST2+ Treg cells and/or CD25^{bright}ST2^{bright} Treg cells. In some embodiments, the bifunctional protein expands non-traditional, Foxp3− immune-regulatory T-cells (e.g., Tr1 cells and Th3 cells). In some embodiments, the Foxp3− immune regulatory T-cells are Tr1 cells. In some embodiments, the Foxp3− immune regulatory T-cells are Th3 cells.

In some aspects, provided herein is a fusion protein comprising, an IL-2 polypeptide, and an IL-33 polypeptide comprising amino acid substitutions of N60S, C97G, and C116F, according to the sequence of SEQ ID NO: 1, wherein the IL-2 polypeptide is linked to the IL-33 polypeptide via a linker.

In some embodiments, the IL-33 polypeptide further comprises an amino acid substitution C148G.

In some embodiments, the IL-33 polypeptide further comprises an amino acid substitution C121S.

In some aspects, provided herein is a fusion protein comprising an IL-2 polypeptide, and an IL-33 polypeptide comprising the amino acid substitution C116F, C116Y or C116A according to the sequence of SEQ ID NO: 21, wherein the IL-2 polypeptide is linked to the IL-33 polypeptide via a linker.

In some embodiments of the fusion protein, the IL-33 polypeptide further comprises amino acid substitutions at positions N60, C97, C121 and C148.

In some embodiments, the IL-33 polypeptide comprises one or more amino acid substitutions N60S, N60D, C97G, C116F, C121S or C148G.

In some embodiments, the IL-33 polypeptide comprises the amino acid substitutions N60S, C97G, and C116F.

In some embodiments, the IL-33 polypeptide further comprises the amino acid substitution C148G.

In some embodiments, the IL-33 polypeptide further comprises the amino acid substitution C121S.

In some embodiments, the IL-2 polypeptide is an IL-2 variant biased to IL2Rα.

In some embodiments, the IL-2 variant comprises one or more amino acid substitutions selected from T3N, T3A, L12G, L12K, L12Q, L12S, Q13G, E15A, E15G, E15S, H16A, H16D, H16E, H16G, H16K, H16M, H16N, H16R, H16S, H16T, H16V, H16Y, L19A, L19D, L19E, L19G, L19N, L19R, L19S, L19T, L19V, D20A, D20E, D20H, D201, D20Y, D20F, D20G, D20T, D20W, M23R, V69A, Q47P, R81A, R81G, R81S, R81T, D84A, D84E, D84G, D84K, D841, D84M, D84Q D84R, D84S, D84T, S87R, N88A, N88D, N88R, N88E, N88I, N88F, N88G, N88M, N88R, N88S, N88V, N88W, V91D, V91E, V91K, V91G, V91N, V91R, V91S, 192K, I92R, E95G, C125A, C125S Q1261, Q126L, and Q126F.

In some embodiments, the IL-2 variant comprises one or more amino acid substitutions selected from V69A, Q47P, N88D and C125S.

In some embodiments, the IL-2 variant comprises the amino acid substitution N88D.

In some embodiments, the IL-2 variant comprises the amino acid substitutions of N88D and C125S.

In some embodiments, the IL-2 variant comprises the amino acid substitutions of V69A, Q47P, N88D and C125S.

In some embodiments, provided herein is the bifunctional protein or the fusion protein, wherein the IL-2 variant or polypeptide comprises an amino acid sequence selected from SEQ ID NO: 3 to SEQ ID NO:20, and wherein the IL-33 variant or polypeptide comprises an amino acid sequence selected from SEQ ID NO: 22 to SEQ ID NO: 28.

In some embodiments, provided herein is the bifunctional protein or the fusion protein, wherein the peptide linker is selected from GGGGS (SEQ ID NO: 29; n repeats of SEQ ID NO: 29, n=1-5), GGGGSGGGGSGGGGS (SEQ ID NO: 31), EAAAK (SEQ ID NO: 32; n repeats of SEQ ID NO: 32, n=1-3), GGGGGGGG (SEQ ID NO: 33), GSGSGSGSGS (SEQ ID NO: 34), GSGSGSGSGSGSGSGSGSGS (SEQ ID NO: 35), GGSGGSGGS (SEQ ID NO: 36), GSGGS (SEQ ID NO: 37), GSSGS (SEQ ID NO: 38), a SEG-linker, a GSAT (SEQ ID NO: 39), SSSSGSSSSG (SEQ ID NO: 40), a flexible 22 amino acid linker LEGSGQGPGSGQGSGSPGSGQG (SEQ ID NO: 41), and GGGGSEAAAK (SEQ ID NO: 42, n=1-2), EGKSSGSGSESKST (SEQ ID NO: 44), GGGGSLVPRGSGGGGS (SEQ ID NO: 45), KESGSVSSEQLAQFRSLD (SEQ ID NO: 46), GGGSEGGGSEGGGSEGGG (SEQ ID NO: 47) or rigid peptide linkers PAPAP (SEQ ID NO: 48), (Ala-Pro)ₙ, AEAAAKEAAAKA (SEQ ID NO: 49), AEAAAKEAAAKEAAAKEAAAKALEAEAAAKEAAAKEAAAKEAAAKA (SEQ ID NO: 50).

In some embodiments, the linker comprises GGGGSGGGGSGGGGS (SEQ ID NO: 31).

In some embodiments, the bifunctional protein or the fusion protein further comprises a signal peptide.

In some embodiments of bifunctional protein or the fusion protein, the IL-33 variant or polypeptide has increased activity and/or manufacturability as compared to human WT IL-33.

In some embodiments of the bifunctional protein, the IL-2 variant has amino acid substitution N88D, and the IL-33 variant has amino acid substitution C116F.

In some embodiments of the bifunctional protein, the IL-2 variant has amino acid substitutions N88D and C125S and the IL-33 variant has amino acid substitutions C97G, C116F, C121S and/or C148G.

In some embodiments, the IL-2 variant is linked to the IL-33 variant by a linker.

In some embodiments, the linker is a peptide linker or a chemical linker.

In some embodiments, the bifunctional protein, upon administration in vivo, results in eosinophil level at less than 5-fold, less than 4 fold, less than 3 fold or less than 2 fold as compared to the level before the treatment.

In some aspects, provided herein is a bifunctional fusion protein comprising an amino acid sequence having at least 85%, 90%, 95%, 96%, 97%, 98%, or 100% identity to any one of the sequences from SEQ ID NO: 54 to SEQ ID NO: 75 and SEQ ID NO: 80 to SEQ ID NO: 81.

In some aspects, provided herein is a bifunctional fusion protein comprising an amino acid sequence having at least 90% identity to SEQ ID NO: 57.

In some embodiments, the bifunctional fusion protein comprises an amino acid sequence having at least 95% identity to SEQ ID NO: 57.

In some embodiments, the bifunctional fusion protein comprises an amino acid sequence having 100% identity to SEQ ID NO: 57.

In some aspects, provided herein is a bifunctional fusion protein comprising an amino acid sequence having at least 90% identity to SEQ ID NO: 61.

In some embodiments, the bifunctional fusion protein comprises an amino acid sequence having at least 95% identity to SEQ ID NO: 61.

In some embodiments, the bifunctional fusion protein comprises an amino acid sequence having 100% identity to SEQ ID NO: 61.

In some aspects, provided herein is a bifunctional fusion protein comprising an amino acid sequence having at least 90% identity to SEQ ID NO: 69.

In some embodiments, the bifunctional fusion protein comprises an amino acid sequence having at least 95% identity to SEQ ID NO: 69.

In some embodiments, the bifunctional fusion protein comprises an amino acid sequence having 100% identity to SEQ ID NO: 69.

In some aspects, provided herein is a bifunctional fusion protein comprising an amino acid sequence having at least 90% identity to SEQ ID NO: 70.

In some embodiments, the bifunctional fusion protein comprises an amino acid sequence having at least 95% identity to SEQ ID NO: 70.

In some embodiments, the bifunctional fusion protein comprises an amino acid sequence having 100% identity to SEQ ID NO: 70.

In some embodiments, the bifunctional fusion protein comprises an amino acid sequence having at least 90% identity to SEQ ID NO: 80.

In some embodiments, the bifunctional fusion protein comprises an amino acid sequence having at least 95% identity to SEQ ID NO: 80.

In some embodiments, the bifunctional fusion protein comprises an amino acid sequence having 100% identity to SEQ ID NO: 80.

In some embodiments, the bifunctional fusion protein comprises an amino acid sequence having at least 90% identity to SEQ ID NO: 81.

In some embodiments, the bifunctional fusion protein comprises an amino acid sequence having at least 95% identity to SEQ ID NO: 81.

In some embodiments, the bifunctional fusion protein comprises an amino acid sequence having 100% identity to SEQ ID NO: 81.

In some embodiments, provided herein is a polynucleotide encoding the bifunctional protein or the fusion protein of the present disclosure.

In some embodiments, provided herein is a composition comprising the bifunctional protein or the fusion protein of the present disclosure.

In some embodiments, provided herein is a pharmaceutical composition comprising the bifunctional protein or the fusion protein of the present disclosure, and at least a pharmaceutically acceptable carrier.

In some embodiments, provided herein is a vector comprising the polynucleotide of the present disclosure.

In some embodiments, the vector is a viral vector or a non-viral vector.

In some embodiments, provided herein is an engineered cell comprising the polynucleotide or the vector of the present disclosure.

In some embodiments, the cell is a mammalian cell.

In some embodiments, the cell is a bacterial cell, yeast cell or insect cell.

In some embodiments, provided herein is a method for modulating regulatory T cells (Treg) in a subject comprising administering to the subject the bifunctional protein or the fusion protein, or the composition of the present disclosure.

In some embodiments, the method expands ST2+ Treg cells, CD25^{bright} Treg cells and/or circulating Foxp3+ Treg and Foxp3-negative Tr1 or Th3 cells.

In some embodiments, the ST2+ Treg cells comprises ST2⁺CD25^{bright} double positive Tregs.

In some embodiments, a proportion of ST2+ Treg and/or ST2⁺CD25^{bright} double positive Tregs traffic to a tissue at the site of disease.

In some embodiments, the method increases immune tolerance in the tissue microenvironment.

In some embodiments, provided herein is a method for promoting local immune homeostasis in a tissue microenvironment to support Treg durability in a subject comprising administering to the subject the bifunctional protein or the fusion protein, or the composition of the present disclosure.

In some embodiments of the method, the fusion protein upregulates ST2+ Treg cells in the tissue microenvironment.

In some embodiments, ST2+ Treg cells comprises ST2⁺CD25^{bright} double positive Treg cells.

In some embodiments, the fusion protein further activates M2 macrophages, eosinophils, type 2 innate lymphoid cells (ILC2), and/or Th17+ Treg cells in the tissue microenvironment.

In some embodiments, the fusion protein inhibits NK cells, M1 macrophages, T effector cells, and/or pro-inflammatory cytokines. In some embodiments, the T effector cells are Th1 cells. In some embodiments, the T effector cells are Th17 cells. In some embodiments, the T effector cells are a combination of Th1 and Th17 cells.

In some embodiments, the fusion protein further decreases immunogenic response in the tissue.

In some embodiments, the tolerogenic tissue environment restores durable response to defend a disease.

In some embodiments, the durable immune response modulates inflammatory suppression in the tissues at the site of disease.

In some aspects, provided herein is a method for selectively expanding ST2+ Treg cells in a subject comprising administering to the subject a bifunctional protein comprising an IL-2 variant associated with an IL-33 variant, wherein the IL-2 variant has amino acid substitutions N88D and the IL-33 variant has amino acid substitution C116F, wherein the administration of the bifunctional protein results in eosinophil level at less than 5 fold, less than 4 fold, less than 3 fold or less than 2 fold as compared to the level before the treatment.

In some embodiments, the IL-2 variant has amino acid substitutions N88D and C125S and the IL-33 variant has amino acid substitutions selected from: (i) C97G and C116F; (ii) C97G, C116F and C148G; and (iii) C97G, C116F, C121S and C148G.

In some embodiments, the administration of the bifunctional protein results in eosinophil levels at less than 3 fold or less than 2 fold as compared to the level before the treatment.

In some embodiments, provided herein is a method of treating an autoimmune or inflammatory disease in a subject comprising administering to the subject the bifunctional protein or the fusion protein, or the composition of the present disclosure.

In some embodiments, the disease is an autoimmune disease.

In some embodiments, provided herein is a population of Treg cells generated by contacting a T cell containing sample the bifunctional protein or the fusion protein, or the composition of the present disclosure.

In some embodiments, the Treg cells are ST2+ Treg cells.

In some embodiments, T cell containing sample is a blood sample, a cell culture-derived cell sample, an iPSC-derived cell sample, an engineered cell sample and/or CAR-Treg cell sample modified to express CD25 and/or ST2.

Any aspect or embodiment described herein can be combined with any other aspect or embodiment as disclosed herein. While the disclosure has been described in conjunction with the detailed description thereof, the foregoing description is intended to illustrate and not limit the scope of the disclosure, which is defined by the scope of the appended claims. Other aspects, advantages, and modifications are within the scope of the following claims.

All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference.

Other features and advantages of the invention will be apparent from the detailed description, drawings and claims that follow. It should be understood, however, that the detailed description, the drawings, and the claims, while indicating embodiments of the present invention, are given by way of illustration only, not limitation. Various changes and modifications within the scope of the invention will become apparent to those skilled in the art.

### BRIEF DESCRIPTION OF THE DRAWINGS

The following figures are for illustration purposes only and not for limitation.

FIG. 1A is a graph showing IL-33 activity of IL-33 WT, IL-33 variant, and a bifunctional protein comprising IL-33 variant and an IL-2 variant as measured by secreted embryonic alkaline phosphatase (SEAP) levels in an IL-33 reporter assay plotted relative to a log of the concentration of IL-33 variant. FIG. 1B is a graph showing IL-2 activity of IL-33 variant, IL-2 WT and a bifunctional protein comprising IL-33 variant and an IL-2 variant as measured by SEAP levels in an IL-2 reporter assay plotted relative to a log of the concentration of IL-2 variant.

FIG. 2A is an SDS PAGE gel showing expression of an exemplary IL233 bifunctional protein, SLT-8, in reducing and non-reducing conditions. The SLT-8 protein purified from CHO cells showed greater than 95% purity. FIG. 2B is chromatogram of an exemplary bifunctional protein purified from bacteria (e.g., E. coli) showing a major peak of 96% purity, comprising no high molecular weight (HMW) species and about 4% low molecular weight (LMW) species. FIG. 2C is a graph showing potency of an exemplary bifunctional protein, SLT8 purified from E. coli as measured by SEAP levels plotted relative to a log of the concentration of the protein in a STAT5− inducible secreted embryonic alkaline phosphatase (SEAP) reporter gene levels in an HEK-Blue™ IL-2 reporter cell line (InvivoGen). From the graph, the EC50 was determined as 0.5972, indicative of high potency. FIG. 2D is a graph showing potency of an exemplary bifunctional protein, SLT8 purified from E. coli as measured by SEAP levels plotted relative to a log of the concentration of the protein in a NF-κB/AP-1− inducible secreted embryonic alkaline phosphatase (SEAP) reporter gene levels in an HEK-Blue™ IL-33 reporter cell line (InvivoGen). From the graph, the EC50 was determined as 0.1787, indicative of high potency.

FIG. 3A shows graphs of potency of exemplary bifunctional protein, SLT8 as measured by SEAP levels plotted relative to a log of the concentration of the protein in a STAT5− inducible secreted embryonic alkaline phosphatase (SEAP) reporter gene levels (indicative of IL-2 signaling) in an HEK-Blue™ IL-2 reporter cell line (InvivoGen) at day 0, and upon storage until day 7 at 37° C., day 28 at 25° C. and day 90 at between 2-8° C. From the graph, the EC50 was determined as 0.9334 at day 0, 1.588 at day 7 upon storage at 37° C., 0.6399 at day 28 upon storage at 25° C. and 0.6271 at day 90 upon storage at between 2-8° C., indicative of maintenance of high potency and stability upon storage, for example, upon storage at 2-8° C. and 25° C. FIG. 3B shows graphs of potency of exemplary bifunctional protein, SLT8 as measured by SEAP levels plotted relative to a log of the concentration of the protein in a NF-κB/AP-1− inducible secreted embryonic alkaline phosphatase (SEAP) reporter gene levels (indicative of IL-33 signaling) in an HEK-Blue™ IL-33 reporter cell line (InvivoGen) at day 0, and upon storage until day 7 at 37° C., day 28 at 25° C. and day 90 at between 2-8° C. From the graph, the EC50 was determined as 0.06481 at day 0, 0.1805 at day 7 upon storage at 37° C., 0.06605 at day 28 upon storage at 25° C. and 0.05220 at day 90 upon storage at between 2-8° C., indicative of maintenance of high potency and stability upon storage, for example, upon storage at 2-8° C. and 25° C.

FIG. 4A is a bar graph showing percentage of regulatory T cells (Tregs) as measured in exemplary bifunctional proteins, SLT4, SLT8, SLT16, SLT17 relative to vehicle control, which was comparable in all the bifunctional proteins shown in this graph. FIG. 4B is a bar graph showing percentage of helper T cells as measured in exemplary bifunctional proteins, SLT4, SLT8, SLT16, SLT17 relative to vehicle control, which was comparable in all the bifunctional proteins shown in this graph. FIG. 4C is a bar graph showing percentage of eosinophils as measured in exemplary bifunctional proteins, SLT4, SLT8, SLT16, SLT17 relative to vehicle control. SLT4 and SLT8 showed a lower relative percentage of eosinophils.

FIG. 5A is a graph showing downstream IL-2 signaling in human PBMCs by measuring phosphorylated STAT5 levels of an exemplary bifunctional protein, SLT4. Whole blood cells from healthy donors were collected and stimulated with SLT4. The pSTAT5 levels were indicative of primarily IL-2 activity. FIG. 5B is a graph showing downstream IL-33 signaling in human PBMCs by measuring phosphorylated p38 levels indicative of activation of MAPK signaling of an exemplary bifunctional protein, SLT4. Whole blood cells from healthy donors were collected and stimulated with SLT4. The p38 levels were indicative of primarily IL-33 activity.

FIG. 6A is a graph showing percentage of ST2+ Tregs in fresh human PBMCs stimulated with an exemplary bifunctional protein, SLT4 or stimulated with IL-2 as a control. FIG. 6B is a graph showing induction of total Treg proliferation by SLT4 as well as the percentage of Treg subsets in SLT4 stimulated PBMCs, comprising mainly ST2+CD25 bright Tregs and ST2+ Tregs. CD25 bright Tregs were induced at higher concentrations of SLT4.

FIG. 7A-7D are bar graphs measuring the percentage or absolute number of total Tregs and ST2+ Tregs in leukocytes isolated from fresh synovial fluid from injured knee. Leukocytes were cultured for 24 hours in the presence of equimolar amounts of human IL-2 or an exemplary bifunctional protein, SLT4. Cells were analyzed by flow cytometry for the proportion and number of total and ST2+ Tregs. FIG. 7A is a bar graph showing percentage of Fox3+ Tregs at various concentrations of SLT4 and IL-2 ranging from 0 ng/ml, 50 ng/ml and 100 ng/ml. The percentage of total Tregs is increased in the SLT4 sample at both 50 ng/ml and 100 ng/ml concentrations. FIG. 7B is a bar graph showing total number of Fox3+ Tregs at various concentrations of SLT4 and IL-2 ranging from 0 ng/ml, 50 ng/ml and 100 ng/ml. The number of total Tregs is increased in the SLT4 sample at both 50 and 100 ng/ml concentrations. FIG. 7C is a bar graph showing percentage of ST2+ Fox3+ Tregs at various concentrations of SLT4 and IL-2 ranging from 0 ng/ml, 50 ng/ml and 100 ng/ml. The percentage of ST2+ Tregs is increased in the SLT4 sample at both 50 ng/ml and 100 ng/ml concentrations. FIG. 7D is a bar graph showing total number of ST2+ Fox3+ Tregs at various concentrations of SLT4 and IL-2 ranging from 0 ng/ml, 50 ng/ml and 100 ng/ml. The number of total Tregs is increased in the SLT4 sample at both 50 ng/ml and 100 ng/ml concentrations.

FIG. 8A is a bar graph showing the percentage of Treg subsets at various concentrations of an exemplary bifunctional protein, SLT4 ranging from 0 μg/kg, 20 μg/kg, 40 μg/kg and 100 μg/kg in healthy non-human primates. The graph shows that tissue targeting Treg subsets are all expanded in a dose-dependent manner, including predominantly ST2+ Tregs. FIG. 8B is a bar graph showing the percentage of Treg subsets at various concentrations of IL-2 ranging from 0 μg/kg, 20 μg/kg, 40 μg/kg and 100 μg/kg in healthy non-human primates. The graph shows that IL-2 is not as effective at expanding tissue targeting Treg subsets, including ST2+ Tregs, as compared to SLT4.

FIG. 9 is a bar graph showing fold change of Treg subsets on day 6 relative to day 0 at a low dose of 20 μg/kg in healthy non-human primates. Baseline levels of Treg cells stimulated by IL-2 treatment are shown for comparison. The graphs show that even at a low dose, an exemplary bifunctional protein, SLT4 shows induction of ST2+ and ST2+CD25 bright Tregs. In addition, CD25+ bright Tregs are also increased.

FIG. 10 is a graph showing fold change of ST2+ Treg cells relative to day 0 at a low dose of 20 μg/kg of SLT4 and IL-2 as a control in a time course from day 0 to day 14. Relative to IL-2, the SLT4 sample showed a greater fold change induction of ST2+ Treg cells starting at day 0 and increasing until day 6, staying consistently high until 10, when it started decreasing and was approaching comparable levels, although still higher than IL-2 by day 14. On day 28, ST2+ Tregs were higher with SLT4 than IL-2 alone. The IL-2 control is indicative of circulating ST2+ Treg cells, while the SLT4 indicates tissue-targeted ST2+ Tregs in inguinal lymph node suggestive of trafficking of ST2+ Treg cells.

FIG. 11 is a bar graph showing fold change of total Treg and Treg subsets by SLT4 relative to IL-2 on day 28 in lymph. Total Tregs were increased by SLT4 relative to IL-2 control. The greatest induction was of ST2+ Treg cells, followed by CD25bright Tregs. ST2+CD25 bright cells induced by SLT4 on day 28 were comparable to IL-2 control.

FIG. 12 is a bar graph showing percentage of ST2+ Treg cells on day 28 in lymph relative to ST2+ circulating systemically on day 6 at a low dose of 20 μg/kg of SLT4 relative to an IL-2 control. The percentage of ST2+ Tregs in inguinal lymph node tissue remains much higher than ST2+ Tregs circulating in blood, indicative of tissue targeting and a sustained and durable expression and response.

FIG. 13 is a bar graph showing fold change expansion of total Tregs and ST2+ Treg subsets relative to other immune cells, such as T effector cells (CD8, CD4), B cells and NK cells, over day 0 in non-human primates. The greatest expansion was of the ST2+ Treg cells at a dose of 100 μg/kg of SLT4, showing selective expansion of ST2+ Tregs in non-human primates without concomitant expansion of effector cell types.

FIG. 14 is a bar graph showing fold change expansion of total Tregs and ST2+ Treg subsets relative to other immune cells, such as T effector cells (CD8, CD4), B cells and NK cells, over day 0, in non-human primates. The greatest expansion was of the ST2+ Treg cells at a dose of 40 μg/kg of SLT4, showing selective expansion of ST2+ Tregs in non-human primates without concomitant expansion of effector cell types.

FIG. 15 is a bar graph of ratios of Tregs to subgroups of T effector cells, i.e., Tregs relative to FN, Tregs relative to TNFα and Tregs relative to IL-17, at 20 μg/kg, 40 μg/kg and 100 μg/kg of SLT4 in non-human primates. The ratios obtained with SLT4 are normalized to ratios obtained with 50 μg/kg of IL-2. The graph indicates selectivity of SLT4 to Tregs in non-human primates.

FIG. 16A-16C shows a comparison of Treg subsets upon SLT4 administration in non-human primates (FIG. 16A) relative to IL-2 (FIG. 16B) and IL-2 Fc fusion (FIG. 16C). SLT4 showed the largest expansion of ST2+ Treg cells followed by ST2+CD25bright and was more effective than IL-2 or IL-2 Fc fusion.

FIG. 17A is an SDS PAGE gel showing expression of an exemplary IL233 bifunctional protein, SLT-27, in reducing and non-reducing conditions. The SLT-27 protein purified from CHO cells showed greater than 99% purity. FIG. 17B is a chromatogram of an exemplary bifunctional protein purified from mammalian culture (e.g., CHO) showing a major peak of 99.45% purity, comprising minimal high molecular weight (HMW) species (about 0.55%) and no low molecular weight (LMW) species. FIG. 17C is a graph showing comparable potency of exemplary bifunctional proteins, SLT8, SLT27 and SLT28 purified from CHO as measured by SEAP levels plotted relative to a log of the concentration of the protein in a STAT5− inducible secreted embryonic alkaline phosphatase (SEAP) reporter gene levels in an HEK-Blue™ IL-2 reporter cell line (InvivoGen). From the graph, the EC50 was determined as 4.494 (SLT8), 1.319 (SLT27) and 0.8233 (SLT28), indicative of high potency. FIG. 17D is a graph showing comparable potency of exemplary bifunctional proteins, SLT8, SLT27 and SLT28 purified from CHO as measured by SEAP levels plotted relative to a log of the concentration of the protein in a NF-κB/AP-1− inducible secreted embryonic alkaline phosphatase (SEAP) reporter gene levels in an HEK-Blue™ IL-33 reporter cell line (InvivoGen). From the graph, the EC50 was determined as 0.1608 (SLT8), 0.07780 (SLT27) and 0.07512 (SLT28), indicative of high potency.

FIG. 18A is a bar graph showing the number of ST2+ Treg cells at day 10, measured from mice subcutaneously injected with a dose of 10 μg/kg or 100 μg/kg of exemplary bifunctional protein, SLT8 or SLT27 at days 0, 1 or 2. Both SLT8 and SLT27 showed a dose-dependent expansion of ST2+ Treg cells by about 5-10 fold. FIG. 18B is a bar graph showing the amount of total Treg cells at day 10, measured from mice subcutaneously injected with a dose of 10 μg/kg or 100 μg/kg of exemplary bifunctional protein, SLT8 or SLT27 at days 0, 1 or 2. Both SLT8 and SLT27 showed a dose-dependent expansion of total Treg cells by about 2-3 fold.

FIG. 19A is a bar graph of the number of eosinophils measured at days 4, 6 and 10 from mice subcutaneously injected with a dose of 10 μg/kg or 100 μg/kg of exemplary bifunctional protein, SLT8 or SLT27 at days 0, 1 or 2. SLT8 and SLT27 showed comparable amounts of eosinophils at the timepoints tested. FIG. 19B is a bar graph of the percentage of T effector cells measured at days 4, 6 and 10 from mice subcutaneously injected with a dose of 10 μg/kg or 100 μg/kg of exemplary bifunctional protein, SLT8 or SLT27 at days 0, 1 or 2. SLT8 and SLT27 showed comparable amounts of T effector cells at the timepoints tested.

FIG. 20A is a bar graph showing aggregation as determined by percentage of high molecular weight products produced from exemplary bifunctional protein, SLT27 on day 0 and after storage for two weeks at either 25° C. or 30° C. From the graph, the percentage of high molecular weight products was determined as 2.09 at day 0, 1.62 upon storage at 25° C. and 1.42 upon storage at 30° C., indicative of maintenance of stability upon storage. FIG. 20B is a graph of percentage of high molecular weight products produced from exemplary bifunctional protein, SLT27 at 0, 2, 4, 6, 8 and 24 hours.

### DEFINITIONS

In order for the present invention to be more readily understood, certain terms are first defined below. Additional definitions for the following terms and other terms are set forth throughout the Specification.

As used in this Specification and the appended claims, the singular forms “a,” “an” and “the” include plural referents unless the context clearly dictates otherwise.

Unless specifically stated or obvious from context, as used herein, the term “or” is understood to be inclusive and covers both “or” and “and”.

The terms “e.g.,” and “i.e.,” as used herein, are used merely by way of example, without limitation intended, and should not be construed as referring only those items explicitly enumerated in the specification.

The terms “or more”, “at least”, “more than”, and the like, e.g., “at least one” are understood to include but not be limited to at least 1, 2, 3, 4, 5, 6, 7, 8, 9, 10, 11, 12, 13, 14, 15, 16, 17, 18, 1920, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more than the stated value. Also included is any greater number or fraction in between.

Conversely, the term “no more than” includes each value less than the stated value. For example, “no more than 100 nucleotides” includes 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, and 0 nucleotides. Also included is any lesser number or fraction in between.

The terms “plurality”, “at least two”, “two or more”, “at least second”, and the like, are understood to include but not limited to at least 2, 3, 4, 5, 6, 7, 8, 9, 10, 11, 12, 13, 14, 15, 16, 17, 18, 1920, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more. Also included is any greater number or fraction in between.

Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps.

Unless specifically stated or evident from context, as used herein, the term “about” is understood as within a range of normal tolerance in the art, for example within 2 standard deviations of the mean. “About” can be understood to be within 10%, 9%, 8%, 7%, 6%, 5%, 4%, 3%, 2%, 1%, 0.5%, 0.1%, 0.05%, 0.01%, or 0.001% of the stated value. Unless otherwise clear from the context, all numerical values provided herein reflects normal fluctuations that can be appreciated by a skilled artisan.

Throughout the specification and drawings, SLT8 is used interchangeably with SLT-8, SLT-008 or SLT008 and they are materially the same. It is to be understood that similar interchangeable use of nomenclature applies to all SLT constructs described herein.

Fusion protein, as used herein, generally refers to a fusion polypeptide molecule comprising an IL-33 molecule and an IL-2 molecule, wherein the components of the fusion protein are linked to each other by peptide-bonds, either directly or through peptide linkers. In some embodiments, the fusion protein also comprises a half-life extension moiety (e.g., an Fc region, PEG, etc.) and a linker.

Fused refers to components that are linked by peptide bonds, either directly or via one or more peptide linkers.

Affinity or binding affinity refers to the strength of the sum total of non-covalent interactions between a single binding site of a molecule and its binding partner. Unless indicated otherwise, as used herein, binding affinity refers to intrinsic binding affinity which reflects a 1:1 interaction between members of a binding pair. The affinity of a molecule X for its partner Y can generally be represented by the dissociation constant (KD), which is the ratio of dissociation and association rate constants (Koff and Kon, respectively). Thus, equivalent affinities may comprise different rate constants, as long as the ratio of the rate constants remains the same. Affinity can be measured by common methods known in the art, including those described herein. A particular method for measuring affinity is Surface Plasmon Resonance (SPR). Specific binding means that the binding is selective for the protein and can be discriminated from unwanted or non-specific interactions. The ability of a protein to bind to a specific binding partner can be measured either through an enzyme-linked immunosorbent assay (ELISA) or other techniques familiar to one of skill in the art, e.g., Surface Plasmon Resonance (SPR) technique (analyzed on a BIAcore instrument), and traditional binding assays. In some embodiments, the protein has a dissociation constant (D) of <1 M, <100 nM, <10 nM, <1 nM, <0.1 nM, <0.01 nM, or <0.001 nM (e.g., 10-8 M or less, e.g., from 10-8 M to 10-13 M, e.g., from 10-9 M to 10-13 M). Reduced binding, for example reduced binding of a cytokine (e.g. IL-2 or IL-33) to an IL-2 or an IL-33 receptor, refers to a decrease in affinity for the respective interaction, as measured for example by SPR. For clarity the term includes also reduction of the affinity to zero (or below the detection limit of the analytic method), i.e., complete abolishment of the interaction. Conversely, increased binding refers to an increase in binding affinity for the respective interaction.

Autoimmune disease refers to a non-malignant disease or disorder arising from and directed against an individual's own tissues. Examples of autoimmune diseases or disorders include, but are not limited to, inflammatory responses such as inflammatory skin diseases including psoriasis and dermatitis (e.g., atopic dermatitis); responses associated with inflammatory bowel disease (such as Crohn's disease and ulcerative colitis); dermatitis; allergic conditions such as eczema and asthma; rheumatoid arthritis; systemic lupus erythematosus (SLE) (including but not limited to lupus nephritis, cutaneous lupus); diabetes mellitus (e.g., type 1 diabetes mellitus or insulin dependent diabetes mellitus); multiple sclerosis and juvenile onset diabetes. Additional examples of autoimmune diseases include, for example, multiple sclerosis (MS), lupus, ankylosing spondylitis, arthritis, colitis, type 1 diabetes, Crohn's disease, heart disease, graft versus host disease, complications from immune response in pregnancy, allergies, rejection of cell or solid organ transplant, Amyotrophic lateral sclerosis (ALS), and myasthenia gravis.

Interleukin-2 or IL-2 as used herein, refers to any native IL-2 from any vertebrate source, including mammals such as primates (e.g., humans) and rodents (e.g., mice and rats), unless otherwise indicated. The term encompasses unprocessed IL-2 as well as any form of IL-2 that results from processing in the cell. The term also encompasses naturally occurring variants of IL-2, e.g., splice variants or allelic variants. The amino acid sequence of an exemplary human IL-2 is shown in SEQ ID NO: 2. Unprocessed human IL-2 additionally comprises an N-terminal 20 amino acid signal peptide (SEQ ID NO: 1), which is absent in the mature IL-2 molecule. Wild-type IL-2 or native IL-2, also termed wild-type IL-2, refers to a naturally occurring IL-2. The sequence of a native human IL-2 molecule is shown in SEQ ID NO: 2.

Interleukin-2 or IL-2 variant as used herein, refers to an IL-2 mutein that comprises one or more amino acid mutations relative to wild-type. In some embodiments, the one or more mutations do not alter IL-2 receptor binding compared to the naturally occurring, native IL-2, such as e.g., a substitution of cysteine at a position corresponding to residue 125 of human IL-2 to alanine. In some embodiments wild-type IL-2 for the purpose of the present invention comprises one or more amino acid substitutions selected from T3N, T3A, L12G, L12K, L12Q, L12S, Q13G, E15A, E15G, E15S, H16A, H16D, H16E, H16G, H16K, H16M, H16N, H16R, H16S, H16T, H16V, H16Y, L19A, L19D, L19E, L19G, L19N, L19R, L19S, L19T, L19V, D20A, D20E, D20H, D201, D20Y, D20F, D20G, D20T, D20W, M23R, V69A, Q47P, R81A, R81G, R81S, R81T, D84A, D84E, D84G, D84K, D841, D84M, D84Q D84R, D84S, D84T, S87R, N88A, N88D, N88R, N88E, N88I, N88F, N88G, N88M, N88R, N88S, N88V, N88W, V91D, V91E, V91K, V91G, V91N, V91R, V91S, 192K, I92R, E95G, C125A, C125S Q1261, Q126L, and Q126F. In some embodiments of the present invention, the IL-2 polypeptide comprises one or more amino acid substitutions selected from V69A, Q47P, N88D, N88R and C125S. In some embodiments, the IL-2 polypeptide comprises amino acid substitutions of N88D and C125S.

CD25 or IL-2 receptor α as used herein, refers to any native CD25 from any vertebrate source, including mammals such as primates (e.g., humans) and rodents (e.g., mice and rats), unless otherwise indicated. The term encompasses “full-length”, unprocessed CD25 as well as any form of CD25 that results from processing in the cell. The term also encompasses naturally occurring variants of CD25, e.g., splice variants or allelic variants. In some embodiments CD25 is human CD25.

High-affinity IL-2 receptor as used herein refers to the heterotrimeric form of the IL-2 receptor, consisting of the receptor γ-subunit (also known as common cytokine receptor γ-subunit, yc, or CD132), the receptor β-subunit (also known as CD122 or p70) and the receptor α-subunit (also known as CD25 or p55).

Intermediate-affinity IL-2 receptor or IL-2 receptor fly refers to the IL-2 receptor including only the γ-subunit and the β-subunit, without the α-subunit (Olejniczak and Kasprzak, Med Sci Monit 14, RA179-189 (2008)).

Regulatory T cell or Treg cell refers to a specialized type of CD4+ T cell that can suppress the responses of other T cells (effector T cells). Treg cells are characterized by expression of CD4, the α-subunit of the IL-2 receptor (CD25), and the transcription factor forkhead box P3 (FOXP3) (Sakaguchi, Annu Rev Immunol 22, 531-62 (2004)) and play a critical role in the induction and maintenance of peripheral self-tolerance to antigens, including those expressed by tumors.

CD4+ T cells means CD4+ T cells other than regulatory T cells. Conventional CD4+ memory T cells are characterized by expression of CD4, CD3, but not FOXP3. Conventional CD4+ memory T cells are a subset of conventional CD4+ T cells, further characterized by lack of expression of CD45RA, in contrast to conventional CD4+ naive T cells which do express CD45RA.

Selective activation of Treg cells is meant activation of Treg cells essentially without concomitant activation of other T cell subsets (such as CD4+T helper cells, CD8+ cytotoxic T cells, NK T cells) or natural killer (NK) cells. Activation may include induction of IL-2 receptor signaling (as measured e.g., by detection of phosphorylated STAT5a), induction of proliferation (as measured e.g., by detection of Ki-67) and/or up-regulation of expression of activation markers (such as e.g., CD25).

Peptide linker refers to a peptide comprising one or more amino acids, typically about 2-20 amino acids. Peptide linkers are known in the art or are described herein, as non-limiting examples, at Table 4.

Potency refers to the amount of a therapeutic agent needed to produce a given effect. Efficacy is the maximal effect that a drug produces irrespective of concentration (dose). Potency refers to the rate of drug-receptor binding and dissociation whereas efficacy refers to the resultant biological response. Potency is calculated by median effective concentration/dose KD, or EC50, or ED50. It refers to the concentration of therapeutic agent needed to activate 50% of the available receptors.

Modification refers to any manipulation of the peptide backbone (e.g., amino acid sequence) or the post-translational modifications (e.g., glycosylation) of a polypeptide.

Amino acid substitution refers to the replacement in a polypeptide of one amino acid with another amino acid. In one embodiment, an amino acid is replaced with another amino acid having similar structural and/or chemical properties, e.g., conservative amino acid replacements.

Conservative amino acid substitutions may be made on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid.

Non-conservative amino acid substitutions will entail exchanging a member of one of these classes for another class. For example, amino acid substitutions can also result in replacing one amino acid with another amino acid having different structural and/or chemical properties, for example, replacing an amino acid from one group (e.g., polar) with another amino acid from a different group (e.g., basic). Amino acid substitutions can be generated using genetic or chemical methods well known in the art. Genetic methods may include site-directed mutagenesis, PCR, gene synthesis and the like. It is contemplated that methods of altering the side chain group of an amino acid by methods other than genetic engineering, such as chemical modification, may also be useful. Various designations may be used herein to indicate the same amino acid substitution. A position N60 be indicated as N60S, 60S, S60, or Asn60Ser.

Percent (%) amino acid sequence identity with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, California, or may be compiled from the source code. The ALIGN-2 program should be compiled for use on a UNIX operating system, including digital UNIX V4.0D. All sequence comparison parameters are set by the ALIGN-2 program and do not vary. In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

Polynucleotide or nucleic acid as used interchangeably herein, refers to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. A sequence of nucleotides may be interrupted by non-nucleotide components. A polynucleotide may comprise modification(s) made after synthesis, such as conjugation to a label.

Percent identity of nucleic acid sequence: By a nucleic acid or polynucleotide having a nucleotide sequence at least, for example, 95% “identity” to a reference nucleotide sequence of the present invention, it is intended that the nucleotide sequence of the polynucleotide is identical to the reference sequence except that the polynucleotide sequence may include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These alterations of the reference sequence may occur at the 5′ or 3′ terminal positions of the reference nucleotide sequence or anywhere between those terminal positions, interspersed either individually among residues in the reference sequence or in one or more contiguous groups within the reference sequence. As a practical matter, whether any particular polynucleotide sequence is at least 80%, 85%, 90%, 95%, 96%, 97%, 98% or 99% identical to a nucleotide sequence of the present invention is determined conventionally using known computer programs, such as the ones discussed above for polypeptides (e.g., ALIGN-2).

Variant as used herein, refers to a mutein comprising one or more amino acid substitutions relative to wild-type sequence. Mutations maybe any mutation described in the present application, or equivalents thereof. Interleukin-2 or IL-2 variant as used herein, refers to an IL-2 mutein that comprises one or more amino acid mutations relative to wild-type. Interleukin-33 or IL-33 variant as used herein, refers to an IL-33 mutein that comprises one or more amino acid mutations relative to wild-type.

Vector as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors”.

Host cell, host cell line, and host cell culture are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. A host cell is any type of cellular system that can be used to generate the fusion proteins of the present invention. Host cells include cultured cells, e.g., mammalian cultured cells, such as CHO cells, BH cells, NSO cells, SP2/0 cells, YO myeloma cells, P3X63 mouse myeloma cells, PER cells, PER.C6 cells or hybridoma cells, bacterial cells yeast cells, insect cells, and plant cells, to name only a few, but also cells comprised within a transgenic animal, transgenic plant or cultured plant or animal tissue.

Effective amount of an agent refers to the amount that is necessary to result in a physiological change in the cell or tissue to which it is administered.

Therapeutically effective amount of an agent, e.g., a pharmaceutical composition, refers to an amount effective, at dosages and for periods of time necessary, to achieve the desired therapeutic or prophylactic result. A therapeutically effective amount of an agent for example eliminates, decreases, delays, minimizes or prevents adverse effects of a disease.

Individual or subject is a mammal (e.g., human) administered an effective dose of the bifunctional protein.

Manufacturability as used herein, in the context of recombinant proteins (e.g. IL-2, IL-33, including variants, and IL233 fusion proteins), refers to biological activity, oxidation, solubility, stability and/or yield for making “druggable” recombinant proteins for therapeutic use.

Stability: As used herein, the term “stable” refers to the ability of the therapeutic agent (e.g., recombinant IL-2, IL-33, and IL233 fusion proteins) to maintain its therapeutic efficacy (e.g., all or the majority of its intended biological activity and/or physiochemical integrity) over extended periods of time. The stability of a therapeutic agent, and the capability of the pharmaceutical composition to maintain stability of such therapeutic agent, may be assessed over extended periods of time (e.g., for at least 1, 3, 6, 12, 18, 24, 30, 36 months or more). The therapeutic agent essentially retains its physical and/or chemical integrity and biological activity upon storage and during processes subjected to the composition or formulation (such as freeze/thaw, mechanical mixing and lyophilization). For example, protein stability can be measured by formation of high molecular weight (HMW) aggregates, loss of enzyme activity, generation of peptide fragments and shift of charge profiles.

Pharmaceutical composition refers to a preparation which is in such form as to permit the biological activity of an active ingredient contained therein to be effective, and which contains no additional components which are unacceptably toxic to a subject to which the formulation would be administered. As used herein the term “pharmaceutical composition” refers to compositions comprising at least one active ingredient and optionally one or more pharmaceutically acceptable excipients.

Pharmaceutically acceptable carrier refers to an ingredient in a pharmaceutical composition, other than an active ingredient, which is nontoxic to a subject. A pharmaceutically acceptable carrier includes, but is not limited to, a buffer, excipient, stabilizer, or preservative.

Regulatory T cells also referred to as T Regulatory cells or Treg cells are a specialized subpopulation of T cells that act to suppress immune response, thereby maintaining homeostasis and self-tolerance. Treg cells prevent excessive inflammation and autoimmune diseases. The most reliable cell-specific marker of Treg is Forkhead box P3 (FOXP3), which is essential for the maturation and function of Treg. The main subsets of CD4+ regulatory T (Tr) cells are the CD4+CD25+ Tr1 cells and the type 1 regulatory (Tr1) cells. Tregs inhibit T cell proliferation and cytokine production and play a critical role in preventing autoimmunity. At the onset of inflammation, Treg cells neutralize inflammatory cytokine secretion (e.g., IL-6, IFN-7, TNF-α, and IL-10). In addition, Treg cells promote apoptosis of neutrophils and encourage phagocytosis of dead neutrophils by macrophages. Tregs further inhibit monocyte activity, survival, and stimulate macrophage polarization toward an anti-inflammatory phenotype (M2) via the release of anti-inflammatory cytokines (e.g., IL-4, IL-10, IL-13). Tregs also suppress CD4 and CD8 T cell-mediated inflammation (via IL-10, TGF-β, and IL-35). Collectively, these Treg-mediated mechanisms result in the inhibition of neutrophil, inflammatory macrophage, CD4 and CD8 T-cell activity, promoting tissue repair and regeneration, leading to Tregs also being called “reparative Tregs” or “suppressive Tregs” or “tissue targeting Tregs”.

ST2+ Treg cells are a subset of Treg cells that contain the ST2 cell surface marker, which is a component of a cytokine receptor also known interleukin 1 receptor-like 1 protein (IL1RL1), which is a subunit of the IL-33 receptor. In addition, immune cells may not show ST2 expression, but cells that are suppressive, tissue resident or reparative subsequent to treatment with the said bifunctional protein, are included in this subset, because ST2 can be internalized and degraded subsequent to engagement with IL-33.

Signal peptide: As used herein, a “signal sequence” or “signal peptide” is a polynucleotide or polypeptide, respectively, which is from about 9 to 200 nucleotides (about 3-66 amino acids) in length, that is incorporated at the 5′ terminus of the coding region or the N-terminus polypeptide encoded, respectively. In some embodiments, addition of these sequences result in trafficking of the encoded polypeptide to the endoplasmic reticulum through one or more secretory pathways. Some signal peptides are cleaved from the protein by signal peptidase after the proteins are transported.

Substantially refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term substantially is therefore used herein to capture the potential lack of completeness inherent in many biological and chemical phenomena.

Synergistic Effect: As used herein, the term “synergistic effect” refers to an overall effect of a biological moiety, such as, for example, a fusion protein, that is greater than the sum of individual effects of the components, such as, the peptides included in the fusion protein. For the present invention, an example of synergistic effect is a greater effect of a bifunctional protein comprising an IL-33 variant and IL-2 than either cytokine by itself.

Treatment (and grammatical variations thereof such as “treat” or “treating”) refers to clinical intervention in an attempt to alter the natural course of a disease in the individual being treated, and can be performed either for prophylaxis or during the course of clinical pathology. Desirable effects of treatment include, but are not limited to, preventing occurrence or recurrence of disease, alleviation of symptoms, diminishing of any direct or indirect pathological consequences of the disease, preventing autoimmune disease, decreasing the rate of disease progression, amelioration or palliation of the disease state, and remission or improved prognosis. In some embodiments, antibodies of the invention are used to delay development of a disease or to slow the progression of a disease.

Unless otherwise defined, all technical and scientific terms used herein have the same meaning as commonly understood by one of ordinary skill in the art to which this application belongs and as commonly used in the art to which this application belongs; such art is incorporated by reference in its entirety. In the case of conflict, the present Specification, including definitions, will control.

### DETAILED DESCRIPTION OF THE INVENTION

The present invention provides, among other things, bifunctional proteins comprising human interleukin-2 (IL-2) and human interleukin-3 (IL-33) variants that are associated together and synergistically activate proliferation of immune cells, for example, regulatory T cells. Without wishing to be bound by any particular theory, it is contemplated that the bifunctional protein disclosed herein improves targeting of the activities of the IL-2 and IL-33 to tissue resident Tregs and other anti-inflammatory cells. The bifunctional protein of the invention can bind to two receptors independently and not necessarily in cis, resulting in a peptide with two separate activities based on the interaction with two different receptors. Disclosed herein are the unexpected results of a synergistic effect of the combination of IL-2 and IL-33 in a single bifunctional protein.

More specifically, IL-2 and IL-33 preferentially expand or stimulate a subset of Treg cells known as ST2+ Treg cells or reparative Treg cells. Without wishing to be bound by any particular theory, it is contemplated that following acute tissue injury (e.g., muscle), large numbers of ST2+ Tregs are rapidly recruited to injured tissues, where among other things, they produce a growth factor amphiregulin (AREG) among expression of several other pro-reparative and resolutionary factors (for example and not limited to IL-10, IL-13, TGFβ, PDGF, Granzyme A/B, Perforin Adenosine, Sphingosine1-phosphate, EGFR, TSLPR, CD39, CD73, PD-1, TIM-3, LAG-3, CTLA-4, Jag-1, Nrp-1, TNFRII, GATA3, BATF, chemokine receptors (adhesion molecules—LFA-1, VLA-4, CD103, αEb7, α4b7, αvb3, αvb8), leading to migration to the sites of injury as well as activation and recruitment of progenitor cells for tissue repair in multiple organ systems. Treatment of ST2+ Treg with the ligand for the ST2 receptor, IL-33, increases AREG production and induce tissue repair. Therefore, enhancing the number of ST2+ Tregs or the activity of ST2+ Tregs by the methods of the present invention treats, or prevents, autoimmune and inflammatory diseases, and improves tissue healing after injury or stress.

By providing compounds and methods that are able to selectively expand ST2+ Tregs numbers and/or enhance ST2+ Treg activity, the present disclosure makes possible new treatments of inflammatory and degenerative diseases. Provided herein are compositions and methods for proliferation and activity of suppressive reparative regulatory Treg cells, which are mobilized to local tissues, resulting in a safe, potent and durable immune response facilitating local tissue microenvironment homeostasis. For example, the present invention provides a bifunctional protein that comprises IL-2 and IL-33, which synergistically expand and localize ST2 cells to tissues.

The present invention provides compositions and methods for selective proliferation of tissue-resident suppressive regulatory T cells, which are also referred to as reparative Treg cells or ST2+ Treg cells. The present invention provides, among other things, compositions and methods for prophylaxis and treatment of autoimmune disease and inflammatory disorders.

The present invention provides, among other things, compositions and methods for prophylaxis and treatment of autoimmune disease, including, as non-limiting examples, Type 1 diabetes (T1D), Systemic Lupus Erythematosus (SLE), Acute Kidney Injury (AKI) and Graft vs. Host Disease (GVHD).

Among other things, the IL-33 variants of the present invention have enhanced manufacturability (e.g., easily expressed recombinantly, show reduced aggregation, leading to improved purification and yield) and/or activity. The variants described herein also showed increased stability (e.g., after storage), increased activity and increased potency.

Various aspects of the invention are described in detail in the following sections. The use of sections is not meant to limit the invention. Each section can apply to any aspect of the invention. In this application, the use of “or” means “and/or” unless stated otherwise.

### Bifunctional Protein Comprising IL-2 and IL-33 Variant (IL233 Variant)

In some aspects, provided herein is a bifunctional protein comprising an IL-2 polypeptide, and an IL-33 variant that has improved activity and/or manufacturability as compared to human WT IL-33, wherein the IL-2 polypeptide is associated with the IL-33 variant, and wherein the bifunctional protein preferentially expands ST2+ Treg cells.

In some embodiments of the bifunctional protein, the IL-2 polypeptide is linked to the IL-33 variant via a peptide linker or a chemical linker. In some embodiments of the bifunctional protein, the IL-2 polypeptide is linked to the IL-33 variant via a peptide linker. In some embodiments of the bifunctional protein, the IL-2 polypeptide is linked to the IL-33 variant via a chemical linker. In some embodiments, the IL-2 is at the N-terminus. In some embodiments, the IL-2 is at the C-terminus. In some embodiments, the IL-33 is at the N-terminus. In some embodiments, the IL-33 is at the C-terminus.

In some embodiments of the bifunctional protein, the linker is a peptide linker comprising 3-30 amino acids. In some embodiments, the peptide linker comprises 3 amino acids. In some embodiments, the peptide linker comprises 4 amino acids. In some embodiments, the peptide linker comprises 5 amino acids. In some embodiments, the peptide linker comprises 6 amino acids. In some embodiments, the peptide linker comprises 7 amino acids. In some embodiments, the peptide linker comprises 8 amino acids. In some embodiments, the peptide linker comprises 9 amino acids. In some embodiments, the peptide linker comprises 10 amino acids. In some embodiments, the peptide linker comprises 11 amino acids. In some embodiments, the peptide linker comprises 12 amino acids. In some embodiments, the peptide linker comprises 13 amino acids. In some embodiments, the peptide linker comprises 14 amino acids. In some embodiments, the peptide linker comprises 15 amino acids. In some embodiments, the peptide linker comprises 16 amino acids. In some embodiments, the peptide linker comprises 17 amino acids. In some embodiments, the peptide linker comprises 18 amino acids. In some embodiments, the peptide linker comprises 19 amino acids. In some embodiments, the peptide linker comprises 20 amino acids. In some embodiments, the peptide linker comprises 21 amino acids. In some embodiments, the peptide linker comprises 22 amino acids. In some embodiments, the peptide linker comprises 23 amino acids. In some embodiments, the peptide linker comprises 24 amino acids. In some embodiments, the peptide linker comprises 25 amino acids. In some embodiments, the peptide linker comprises 26 amino acids. In some embodiments, the peptide linker comprises 27 amino acids. In some embodiments, the peptide linker comprises 28 amino acids. In some embodiments, the peptide linker comprises 29 amino acids. In some embodiments, the peptide linker comprises 30 amino acids.

In some aspects, provided herein is a bifunctional protein comprising an IL-2 polypeptide and an IL-33 polypeptide, wherein the IL-2 polypeptide and the IL-33 polypeptide are associated with each other at a distance of between 1 nm to 10 nm. In some aspects, provided herein is a bifunctional protein comprising an IL-2 polypeptide and an IL-33 polypeptide, wherein the IL-2 polypeptide and the IL-33 polypeptide are associated with each other at a distance of 1 nm. In some aspects, provided herein is a bifunctional protein comprising an IL-2 polypeptide and an IL-33 polypeptide, wherein the IL-2 polypeptide and the IL-33 polypeptide are associated with each other at a distance of 2 nm. In some aspects, provided herein is a bifunctional protein comprising an IL-2 polypeptide and an IL-33 polypeptide, wherein the IL-2 polypeptide and the IL-33 polypeptide are associated with each other at a distance of 3 nm. In some aspects, provided herein is a bifunctional protein comprising an IL-2 polypeptide and an IL-33 polypeptide, wherein the IL-2 polypeptide and the IL-33 polypeptide are associated with each other at a distance of 4 nm. In some aspects, provided herein is a bifunctional protein comprising an IL-2 polypeptide and an IL-33 polypeptide, wherein the IL-2 polypeptide and the IL-33 polypeptide are associated with each other at a distance of 5 nm. In some aspects, provided herein is a bifunctional protein comprising an IL-2 polypeptide and an IL-33 polypeptide, wherein the IL-2 polypeptide and the IL-33 polypeptide are associated with each other at a distance of 6 nm. In some aspects, provided herein is a bifunctional protein comprising an IL-2 polypeptide and an IL-33 polypeptide, wherein the IL-2 polypeptide and the IL-33 polypeptide are associated with each other at a distance of 7 nm. In some aspects, provided herein is a bifunctional protein comprising an IL-2 polypeptide and an IL-33 polypeptide, wherein the IL-2 polypeptide and the IL-33 polypeptide are associated with each other at a distance of 8 nm. In some aspects, provided herein is a bifunctional protein comprising an IL-2 polypeptide and an IL-33 polypeptide, wherein the IL-2 polypeptide and the IL-33 polypeptide are associated with each other at a distance of 9 nm. In some aspects, provided herein is a bifunctional protein comprising an IL-2 polypeptide and an IL-33 polypeptide, wherein the IL-2 polypeptide and the IL-33 polypeptide are associated with each other at a distance of 10 nm.

In some aspects, provided herein is a bifunctional protein comprising an IL-2 polypeptide and an IL-33 polypeptide, wherein the IL-2 polypeptide and the IL-33 polypeptide are associated with each other at a distance of between 10 Å to 100 Å. In some aspects, provided herein is a bifunctional protein comprising an IL-2 polypeptide and an IL-33 polypeptide, wherein the IL-2 polypeptide and the IL-33 polypeptide are associated with each other at a distance of 10 Å. In some aspects, provided herein is a bifunctional protein comprising an IL-2 polypeptide and an IL-33 polypeptide, wherein the IL-2 polypeptide and the IL-33 polypeptide are associated with each other at a distance of 20 Å. In some aspects, provided herein is a bifunctional protein comprising an IL-2 polypeptide and an IL-33 polypeptide, wherein the IL-2 polypeptide and the IL-33 polypeptide are associated with each other at a distance of 30 Å. In some aspects, provided herein is a bifunctional protein comprising an IL-2 polypeptide and an IL-33 polypeptide, wherein the IL-2 polypeptide and the IL-33 polypeptide are associated with each other at a distance of 40 Å. In some aspects, provided herein is a bifunctional protein comprising an IL-2 polypeptide and an IL-33 polypeptide, wherein the IL-2 polypeptide and the IL-33 polypeptide are associated with each other at a distance of 50 Å. In some aspects, provided herein is a bifunctional protein comprising an IL-2 polypeptide and an IL-33 polypeptide, wherein the IL-2 polypeptide and the IL-33 polypeptide are associated with each other at a distance of 60 Å. In some aspects, provided herein is a bifunctional protein comprising an IL-2 polypeptide and an IL-33 polypeptide, wherein the IL-2 polypeptide and the IL-33 polypeptide are associated with each other at a distance of 70 Å. In some aspects, provided herein is a bifunctional protein comprising an IL-2 polypeptide and an IL-33 polypeptide, wherein the IL-2 polypeptide and the IL-33 polypeptide are associated with each other at a distance of 80 Å. In some aspects, provided herein is a bifunctional protein comprising an IL-2 polypeptide and an IL-33 polypeptide, wherein the IL-2 polypeptide and the IL-33 polypeptide are associated with each other at a distance of 90 Å. In some aspects, provided herein is a bifunctional protein comprising an IL-2 polypeptide and an IL-33 polypeptide, wherein the IL-2 polypeptide and the IL-33 polypeptide are associated with each other at a distance of 100 Å.

In some embodiments of the bifunctional protein, the distance is about 1.5 nm to 10 nm, 3.5 nm to 10 nm, 3.5 nm to 7 nm, 5 nm to 10 nm, or 5 nm to 7 nm. In some embodiments of the bifunctional protein, the distance is about 1.5 nm to 10 nm. In some embodiments of the bifunctional protein, the distance is about 1.5 nm. In some embodiments of the bifunctional protein, the distance is about 2 nm. In some embodiments of the bifunctional protein, the distance is about 2.5 nm. In some embodiments of the bifunctional protein, the distance is about 3 nm. In some embodiments of the bifunctional protein, the distance is about 3.5 nm. In some embodiments of the bifunctional protein, the distance is about 4 nm. In some embodiments of the bifunctional protein, the distance is about 4.5 nm. In some embodiments of the bifunctional protein, the distance is about 5 nm. In some embodiments of the bifunctional protein, the distance is about 5.5 nm. In some embodiments of the bifunctional protein, the distance is about 6 nm. In some embodiments of the bifunctional protein, the distance is about 6.5 nm. In some embodiments of the bifunctional protein, the distance is about 7 nm. In some embodiments of the bifunctional protein, the distance is about 7.5 nm. In some embodiments of the bifunctional protein, the distance is about 8 nm. In some embodiments of the bifunctional protein, the distance is about 8.5 nm. In some embodiments of the bifunctional protein, the distance is about 9 nm. In some embodiments of the bifunctional protein, the distance is about 9.5 nm. In some embodiments of the bifunctional protein, the distance is about 10 nm.

In some embodiments of the bifunctional protein, the distance is about 15 Å to 100 Å, 35 Å to 100 Å, 35 Å to 70 Å, 50 Å to 100 Å, or 50 Å to 70 Å. In some embodiments of the bifunctional protein, the distance is about 15 Å to 100 Å. In some embodiments of the bifunctional protein, the distance is about 15 Å. In some embodiments of the bifunctional protein, the distance is about 20 Å. In some embodiments of the bifunctional protein, the distance is about 15 Å to 25 Å. In some embodiments of the bifunctional protein, the distance is about 30 Å. In some embodiments of the bifunctional protein, the distance is about 35 Å. In some embodiments of the bifunctional protein, the distance is about 40 Å. In some embodiments of the bifunctional protein, the distance is about 45 Å. In some embodiments of the bifunctional protein, the distance is about 50 Å. In some embodiments of the bifunctional protein, the distance is about 55 Å. In some embodiments of the bifunctional protein, the distance is about 60 Å. In some embodiments of the bifunctional protein, the distance is about 65 Å. In some embodiments of the bifunctional protein, the distance is about 70 Å. In some embodiments of the bifunctional protein, the distance is about 75 Å. In some embodiments of the bifunctional protein, the distance is about 80 Å. In some embodiments of the bifunctional protein, the distance is about 85 Å. In some embodiments of the bifunctional protein, the distance is about 90 Å. In some embodiments of the bifunctional protein, the distance is about 95 Å. In some embodiments of the bifunctional protein, the distance is about 100 Å.

In some embodiments of the bifunctional protein, the IL-2 polypeptide and the IL-33 polypeptide are linked by a linker.

In some embodiments, the linker is a peptide linker or a chemical linker. In some embodiments, the linker is a peptide linker. In some embodiments, the linker is a chemical linker.

In some aspects, provided herein is a bifunctional protein comprising from the N-terminus to the C-terminus, an IL-2 polypeptide, a linker, and an IL-33 variant that has improved activity and/or manufacturability as compared to human WT IL-33, wherein the linker comprises 3-30 amino acids. In some aspects, provided herein is a bifunctional protein comprising from the N-terminus to the C-terminus, an IL-2 polypeptide, a linker, and an IL-33 variant that has improved activity and/or manufacturability as compared to human WT IL-33, wherein the linker comprises 3 amino acids. In some aspects, provided herein is a bifunctional protein comprising from the N-terminus to the C-terminus, an IL-2 polypeptide, a linker, and an IL-33 variant that has improved activity and/or manufacturability as compared to human WT IL-33, wherein the linker comprises 5 amino acids. In some aspects, provided herein is a bifunctional protein comprising from the N-terminus to the C-terminus, an IL-2 polypeptide, a linker, and an IL-33 variant that has improved activity and/or manufacturability as compared to human WT IL-33, wherein the linker comprises 6 amino acids. In some aspects, provided herein is a bifunctional protein comprising from the N-terminus to the C-terminus, an IL-2 polypeptide, a linker, and an IL-33 variant that has improved activity and/or manufacturability as compared to human WT IL-33, wherein the linker comprises 7 amino acids. In some aspects, provided herein is a bifunctional protein comprising from the N-terminus to the C-terminus, an IL-2 polypeptide, a linker, and an IL-33 variant that has improved activity and/or manufacturability as compared to human WT IL-33, wherein the linker comprises 8 amino acids. In some aspects, provided herein is a bifunctional protein comprising from the N-terminus to the C-terminus, an IL-2 polypeptide, a linker, and an IL-33 variant that has improved activity and/or manufacturability as compared to human WT IL-33, wherein the linker comprises 9 amino acids. In some aspects, provided herein is a bifunctional protein comprising from the N-terminus to the C-terminus, an IL-2 polypeptide, a linker, and an IL-33 variant that has improved activity and/or manufacturability as compared to human WT IL-33, wherein the linker comprises 10 amino acids. In some aspects, provided herein is a bifunctional protein comprising from the N-terminus to the C-terminus, an IL-2 polypeptide, a linker, and an IL-33 variant that has improved activity and/or manufacturability as compared to human WT IL-33, wherein the linker comprises 11 amino acids. In some aspects, provided herein is a bifunctional protein comprising from the N-terminus to the C-terminus, an IL-2 polypeptide, a linker, and an IL-33 variant that has improved activity and/or manufacturability as compared to human WT IL-33, wherein the linker comprises 12 amino acids. In some aspects, provided herein is a bifunctional protein comprising from the N-terminus to the C-terminus, an IL-2 polypeptide, a linker, and an IL-33 variant that has improved activity and/or manufacturability as compared to human WT IL-33, wherein the linker comprises 13 amino acids. In some aspects, provided herein is a bifunctional protein comprising from the N-terminus to the C-terminus, an IL-2 polypeptide, a linker, and an IL-33 variant that has improved activity and/or manufacturability as compared to human WT IL-33, wherein the linker comprises 14 amino acids. In some aspects, provided herein is a bifunctional protein comprising from the N-terminus to the C-terminus, an IL-2 polypeptide, a linker, and an IL-33 variant that has improved activity and/or manufacturability as compared to human WT IL-33, wherein the linker comprises 15 amino acids. In some aspects, provided herein is a bifunctional protein comprising from the N-terminus to the C-terminus, an IL-2 polypeptide, a linker, and an IL-33 variant that has improved activity and/or manufacturability as compared to human WT IL-33, wherein the linker comprises 16 amino acids. In some aspects, provided herein is a bifunctional protein comprising from the N-terminus to the C-terminus, an IL-2 polypeptide, a linker, and an IL-33 variant that has improved activity and/or manufacturability as compared to human WT IL-33, wherein the linker comprises 17 amino acids. In some aspects, provided herein is a bifunctional protein comprising from the N-terminus to the C-terminus, an IL-2 polypeptide, a linker, and an IL-33 variant that has improved activity and/or manufacturability as compared to human WT IL-33, wherein the linker comprises 18 amino acids. In some aspects, provided herein is a bifunctional protein comprising from the N-terminus to the C-terminus, an IL-2 polypeptide, a linker, and an IL-33 variant that has improved activity and/or manufacturability as compared to human WT IL-33, wherein the linker comprises 19 amino acids. In some aspects, provided herein is a bifunctional protein comprising from the N-terminus to the C-terminus, an IL-2 polypeptide, a linker, and an IL-33 variant that has improved activity and/or manufacturability as compared to human WT IL-33, wherein the linker comprises 20 amino acids. In some aspects, provided herein is a bifunctional protein comprising from the N-terminus to the C-terminus, an IL-2 polypeptide, a linker, and an IL-33 variant that has improved activity and/or manufacturability as compared to human WT IL-33, wherein the linker comprises 21 amino acids. In some aspects, provided herein is a bifunctional protein comprising from the N-terminus to the C-terminus, an IL-2 polypeptide, a linker, and an IL-33 variant that has improved activity and/or manufacturability as compared to human WT IL-33, wherein the linker comprises 22 amino acids. In some aspects, provided herein is a bifunctional protein comprising from the N-terminus to the C-terminus, an IL-2 polypeptide, a linker, and an IL-33 variant that has improved activity and/or manufacturability as compared to human WT IL-33, wherein the linker comprises 23 amino acids. In some aspects, provided herein is a bifunctional protein comprising from the N-terminus to the C-terminus, an IL-2 polypeptide, a linker, and an IL-33 variant that has improved activity and/or manufacturability as compared to human WT IL-33, wherein the linker comprises 24 amino acids. In some aspects, provided herein is a bifunctional protein comprising from the N-terminus to the C-terminus, an IL-2 polypeptide, a linker, and an IL-33 variant that has improved activity and/or manufacturability as compared to human WT IL-33, wherein the linker comprises 25 amino acids. In some aspects, provided herein is a bifunctional protein comprising from the N-terminus to the C-terminus, an IL-2 polypeptide, a linker, and an IL-33 variant that has improved activity and/or manufacturability as compared to human WT IL-33, wherein the linker comprises 26 amino acids. In some aspects, provided herein is a bifunctional protein comprising from the N-terminus to the C-terminus, an IL-2 polypeptide, a linker, and an IL-33 variant that has improved activity and/or manufacturability as compared to human WT IL-33, wherein the linker comprises 27 amino acids. In some aspects, provided herein is a bifunctional protein comprising from the N-terminus to the C-terminus, an IL-2 polypeptide, a linker, and an IL-33 variant that has improved activity and/or manufacturability as compared to human WT IL-33, wherein the linker comprises 28 amino acids. In some aspects, provided herein is a bifunctional protein comprising from the N-terminus to the C-terminus, an IL-2 polypeptide, a linker, and an IL-33 variant that has improved activity and/or manufacturability as compared to human WT IL-33, wherein the linker comprises 29 amino acids. In some aspects, provided herein is a bifunctional protein comprising from the N-terminus to the C-terminus, an IL-2 polypeptide, a linker, and an IL-33 variant that has improved activity and/or manufacturability as compared to human WT IL-33, wherein the linker comprises 30 amino acids.

In some embodiments of the bifunctional protein, the IL-33 variant or polypeptide is a truncated IL-33 and comprises at least one amino acid substitution at positions selected from N60, C97, C116, C121, and C148 according to the sequence of SEQ ID NO: 21. In some embodiments of the bifunctional protein, the IL-33 variant or polypeptide is a truncated IL-33 and comprises at least one amino acid substitution at position N60 according to the sequence of SEQ ID NO: 21. In some embodiments of the bifunctional protein, the IL-33 variant or polypeptide is a truncated IL-33 and comprises at least one amino acid substitution at position C97 according to the sequence of SEQ ID NO: 21. In some embodiments of the bifunctional protein, the IL-33 variant or polypeptide is a truncated IL-33 and comprises at least one amino acid substitution at position C116 according to the sequence of SEQ ID NO: 21. In some embodiments of the bifunctional protein, the IL-33 variant or polypeptide is a truncated IL-33 and comprises at least one amino acid substitution at position C121 according to the sequence of SEQ ID NO: 21. In some embodiments of the bifunctional protein, the IL-33 variant or polypeptide is a truncated IL-33 and comprises at least one amino acid substitution at position C148 according to the sequence of SEQ ID NO: 21.

In some embodiments, the IL-33 variant or polypeptide comprises two, three, four or five amino acid substitutions selected from positions of N60, C97, C116, C121 and C148. In some embodiments, the IL-33 variant or polypeptide comprises one amino acid substitution at N60. In some embodiments, the IL-33 variant or polypeptide comprises one amino acid substitution at C97. In some embodiments, the IL-33 variant or polypeptide comprises one amino acid substitution at C116. In some embodiments, the IL-33 variant or polypeptide comprises one amino acid substitution at C121. In some embodiments, the IL-33 variant or polypeptide comprises one amino acid substitution at C148. In some embodiments, the IL-33 variant or polypeptide comprises two amino acid substitutions at positions of N60 and C97. In some embodiments, the IL-33 variant or polypeptide comprises two amino acid substitutions at positions of N60 and C116. In some embodiments, the IL-33 variant or polypeptide comprises two amino acid substitutions at positions of N60 and C121. In some embodiments, the IL-33 variant or polypeptide comprises two amino acid substitutions at positions of N60 and C148. In some embodiments, the IL-33 variant or polypeptide comprises two amino acid substitutions at positions of C97 and C116. In some embodiments, the IL-33 variant or polypeptide comprises two amino acid substitutions at positions of C97 and C121. In some embodiments, the IL-33 variant or polypeptide comprises two amino acid substitutions at positions of C97 and C148. In some embodiments, the IL-33 variant or polypeptide comprises two amino acid substitutions at positions of C116 and C121. In some embodiments, the IL-33 variant or polypeptide comprises two amino acid substitutions at positions of C116 and C148. In some embodiments, the IL-33 variant or polypeptide comprises two amino acid substitutions at positions of C121 and C148. In some embodiments, the IL-33 variant or polypeptide comprises three amino acid substitutions. In some embodiments, the IL-33 variant or polypeptide comprises four amino acid substitutions at N60, C97 and C116. In some embodiments, the IL-33 variant or polypeptide comprises four amino acid substitutions at N60, C97 and C121. In some embodiments, the IL-33 variant or polypeptide comprises four amino acid substitutions at N60, C97 and C148. In some embodiments, the IL-33 variant or polypeptide comprises four amino acid substitutions at N60, C116 and C121. In some embodiments, the IL-33 variant or polypeptide comprises four amino acid substitutions at N60, C116 and C148. In some embodiments, the IL-33 variant or polypeptide comprises four amino acid substitutions at N60, C121 and C148. In some embodiments, the IL-33 variant or polypeptide comprises four amino acid substitutions at N60, C97, C116 and C121. In some embodiments, the IL-33 variant or polypeptide comprises four amino acid substitutions at N60, C97, C116 and C148. In some embodiments, the IL-33 variant or polypeptide comprises four amino acid substitutions at N60, C97, C121 and C148. In some embodiments, the IL-33 variant or polypeptide comprises four amino acid substitutions at N60, C116, C121 and C148. In some embodiments, the IL-33 variant or polypeptide comprises four amino acid substitutions at C97, C116, C121 and C148. In some embodiments, the IL-33 variant or polypeptide comprises five amino acid substitutions at N60, C97, C116, C121 and C148. In some embodiments, the IL-33 variant or polypeptide comprises an amino acid substitution at positions N60, wherein the substitution is N60S, N60T, N60Q, N60D, N60E or N60A.

In some embodiments, the IL-33 variant or polypeptide comprises one amino acid substitution selected from the group consisting of N60S(T, Q, D, E, A), C97G(A,V), and C116F(Y, A). In some embodiments, the IL-33 variant or polypeptide comprises an amino acid substitution of N60S. In some embodiments, the IL-33 variant or polypeptide comprises an amino acid substitution of N60T. In some embodiments, the IL-33 variant or polypeptide comprises an amino acid substitution of N60Q. In some embodiments, the IL-33 variant or polypeptide comprises an amino acid substitution of N60D. In some embodiments, the IL-33 variant or polypeptide comprises an amino acid substitution of N60E. In some embodiments, the IL-33 variant or polypeptide comprises an amino acid substitution of N60A.

In some embodiments, the IL-33 variant or polypeptide comprises an amino acid substitution at positions C97, wherein the substitution is selected from C97G, C97A or C97V. In some embodiments, the IL-33 variant or polypeptide comprises an amino acid substitution of C97G. In some embodiments, the IL-33 variant or polypeptide comprises an amino acid substitution of C97A. In some embodiments, the IL-33 variant or polypeptide comprises an amino acid substitution of C97V.

In some embodiments, the IL-33 variant or polypeptide comprises an amino acid substitution at position C116, wherein the substitution is C116F, C116Y or C116A. In some embodiments, the IL-33 variant or polypeptide comprises an amino acid substitution of C116F. In some embodiments, the IL-33 variant or polypeptide comprises an amino acid substitution of C116Y. In some embodiments, the IL-33 variant or polypeptide comprises an amino acid substitution of C116A.

In some embodiments, the IL-33 variant or polypeptide comprises an amino acid substitution of C121S or C121A. In some embodiments, the IL-33 variant or polypeptide comprises an amino acid substitution of C121S. In some embodiments, the IL-33 variant or polypeptide comprises an amino acid substitution of C121A.

In some embodiments, the IL-33 variant or polypeptide comprises an amino acid substitution of C148G, C148S, C148Y, C148N or C148A. In some embodiments, the IL-33 variant or polypeptide comprises an amino acid substitution of C148G. In some embodiments, the IL-33 variant or polypeptide comprises an amino acid substitution of C148S. In some embodiments, the IL-33 variant or polypeptide comprises an amino acid substitution of C148Y. In some embodiments, the IL-33 variant or polypeptide comprises an amino acid substitution of C148N. In some embodiments, the IL-33 variant or polypeptide comprises an amino acid substitution of C148A.

In some embodiments, the IL-33 variant or polypeptide comprises two amino acid substitutions selected from the group consisting of N60S, C97G and C116F. In some embodiments, the IL-33 variant or polypeptide comprises two amino acid substitutions of N60S and C97G. In some embodiments, the IL-33 variant or polypeptide comprises two amino acid substitutions of N60S and C116F. In some embodiments, the IL-33 variant or polypeptide comprises two amino acid substitutions of C97G and C116F.

In some embodiments, the IL-33 variant or polypeptide comprises the amino acid substitutions of N60S, C97G and C116F.

In some embodiments, the IL-33 variant or polypeptide further comprises the amino acid substitution C148G.

In some embodiments, the IL-33 variant or polypeptide further comprises the amino acid substitution C121S.

In some embodiments, the IL-2 polypeptide comprises one or more amino acid substitutions selected from T3N, T3A, L12G, L12K, L12Q, L12S, Q13G, E15A, E15G, E15S, H16A, H16D, H16E, H16G, H16K, H16M, H16N, H16R, H16S, H16T, H16V, H16Y, L19A, L19D, L19E, L19G, L19N, L19R, L19S, L19T, L19V, D20A, D20E, D20H, D201, D20Y, D20F, D20G, D20T, D20W, M23R, V69A, Q47P, R81A, R81G, R81S, R81T, D84A, D84E, D84G, D84K, D841, D84M, D84Q D84R, D84S, D84T, S87R, N88A, N88D, N88R, N88E, N88I, N88F, N88G, N88M, N88R, N88S, N88V, N88W, V91D, V91E, V91K, V91G, V91N, V91R, V91S, 192K, I92R, E95G, C125A, C125S Q1261, Q126L, and Q126F. It is to be understood that the present disclosure includes IL-2 variants that comprise any number and any combination of the amino acid substitutions listed above.

In some embodiments, the IL-2 polypeptide comprises one or more amino acid substitutions selected from V69A, Q47P, N88D, N88R and C125S.

In some embodiments, the IL-2 polypeptide comprises amino acid substitutions of N88D and C125S.

In some embodiments, the bifunctional protein is capable of binding to CD25 and ST2 on the surface of a single cell. In some embodiments, the bifunctional protein is capable of binding to CD25 and ST2 on the surface of a single cell at the same time. In some embodiments, the bifunctional protein is capable of binding to CD25 and ST2 on the surface of a single cell at different times.

In some embodiments, the bifunctional protein is capable of binding to CD25 and ST2 on the surface of different cells.

In some embodiments, the bifunctional protein expands circulating FoxP3+ Treg cells, CD25^{bright} Treg cells, tissue targeting ST2+ Treg cells and/or CD25^{bright}ST2^{bright}Treg cells. In some embodiments, the bifunctional protein expands circulating FoxP3+ Treg cells. In some embodiments, the bifunctional protein expands circulating CD25^{bright} Treg cells. In some embodiments, the bifunctional protein expands circulating tissue targeting ST2+ Treg cells. In some embodiments, the bifunctional protein expands circulating CD25^{bright}ST2^{bright}Treg cells. In some embodiments, the bifunctional protein expands Foxp3− cells. In some embodiments, the Foxp3− cells are Tr1 cells. In some embodiments, the Foxp3− cells are Th3 cells.

### Interleukin-2 (IL-2)

In some embodiments of the bifunctional fusion protein, the interleukin-2 (IL-2) polypeptide is a wild-type IL-2 polypeptide. Interleukin-2 (IL2) is a Type 1 immunostimulatory cytokine for key immune cells including T cells and natural killer (NK) cells. Systemic IL-2 supplementation enhances immunity in a variety of diseases ranging from neoplasms to viral infection. However, its systemic use is restricted by its serious side effects and its efficacy may be limited by a low half-life in vivo of about 10 minutes. IL-2 signaling is mediated through interactions with a high affinity multi-subunit receptor complex. IL-2 is secreted as a single glycosylated polypeptide, and cleavage of a signal sequence is required for its activity. Structurally, IL-2 comprises a bundle of 4 helices (termed A-D), flanked by 2 shorter helices and several loop regions. Residues in helix A, and in the loop region between helices A and B, are important for receptor binding. Secondary structure analysis has suggested similarity to IL-4 and granulocyte-macrophage colony stimulating factor (GMCSF).

IL-2R is a heterotrimeric protein expressed on a variety of different immune cell types, including T cells, NK cells, eosinophils, and monocytes. This broad expression pattern provides a pleiotropic effect on the immune system and a high systemic toxicity of IL-2 treatments, which makes targeting IL-2R+ cells challenging.

IL2-R has three forms, generated by different combinations of three different IL-2R proteins: α (alpha), β (beta), and γ (gamma). These receptor chains assemble to generate the three different receptor forms: (1) the low affinity receptor, IL2Rα, which does not signal; (2) the intermediate affinity receptor (IL2Rβγ), composed of IL2Rβ and IL2Rγ, which is broadly expressed on CD4+ T cells, NK cells, eosinophils, and monocytes; and (3) the high affinity receptor (IL2Rαβγ), composed of IL2Rα, IL2Rβ, and IL2Rγ, which is expressed transiently on activated T cells and constitutively on Treg cells. Mutations in IL-2 can change the binding affinity of IL-2 to different IL-2R receptor forms. Thus, the present disclosure provides compounds that selectively activate and expand Tregs, for example ST2+ Tregs, by synergistically increasing binding of IL-2 to its alpha receptor, and also including an IL-33 variants. An exemplary moiety includes, but is not limited to, an IL-2 variant comprising one or more mutations that modifies the binding relative to WT.

In some embodiments of the bifunctional fusion protein, the IL-2 polypeptide is a variant IL-2 or IL-2 mutein.

### IL-2 Variants

Described herein are various IL-2 variants, interchangeably referred to as IL-2 muteins or modified IL-2, used to expand proliferation and/or activity of ST2+ Treg cells. The IL-2 variants described herein are used to treat autoimmune disease.

In some embodiments, the IL-2 polypeptide is an IL-2 variant biased to IL2Rα, i.e., the invention provides, among other things, immunosuppressive IL-2 variants that have a higher affinity for IL-2Ra than wild-type IL-2. In some embodiments, IL-2 variants contain one or more mutations in positions of the IL-2 sequence that either contact IL-2Rα or alter the orientation of other positions contacting IL-2Rα, resulting in higher affinity for IL-2Rα. The mutations may be in or near areas known to be in close proximity to IL-2Rα based on crystal structures.

In some embodiments, the invention provides immunosuppressive IL-2 variants that have a lower affinity for IL-2RO than wild-type IL-2.

Immunosuppressive IL-2 variants also include variants that demonstrate altered signaling through certain pathways activated by wild-type IL-2 via the IL-2R and result in preferential proliferation/survival/activation of ST2+ cells, e.g., ST2+T-reg cells. Signaling molecules known to be phosphorylated upon activation of the IL-2R include, for example, STAT5, p38, ERK, SYK, LCK, AKT and mTOR.

In some embodiments, as used herein, IL-2 variants suitable for the present invention include any wild-type and modified IL-2 variants (e.g., IL-2 proteins with amino acid mutations, deletions, insertions, and/or fusion proteins) that retain substantial IL-2 biological activity. Typically, a recombinant IL-2 protein is produced using recombinant technology. However, IL-2 proteins (wild-type or modified) purified from natural resources or synthesized chemically can be used according to the present invention.

IL-2 variants (or “IL-2 muteins”) comprise a sequence of amino acids at least 70%, at least 75%, at least 80%, at least 85%, at least 90%, at least 91%, at least 92%, at least 93% at least 94%, at least 95%, at least 96%, at least 97%, at least 98%, at least 99% identical to wild-type IL-2. Further, IL-2 variants include a sequence of amino acids at least 70%, at least 75%, at least 80%, at least 85%, at least 90%, at least 91%, at least 92%, at least 93% at least 94%, at least 95%, at least 96%, at least 97%, at least 98%, at least 99% identical to a functional fragment of wild-type IL-2.

Variants may contain one or more substitutions, deletions, or insertions within the wild-type IL-2 amino acid sequence. Residues are designated herein by the one letter amino acid code followed by the IL-2 amino acid position. Substitutions are designated herein by the one letter amino acid code followed by the IL-2 amino acid position followed by the substituting one letter amino acid code.

In some embodiments, the present invention provides human interleukin-2 variants comprising at least one amino acid substitution that can selectively activate proliferation of regulatory T cells.

In some embodiments, the IL-2 variant comprises one or more amino acid substitutions selected from in relation to the wild-type IL-2 selected from a group consisting of T3N, T3A, L12G, L12K, L12Q, L12S, Q13G, E15A, E15G, E15S, H16A, H16D, H16E, H16G, H16K, H16M, H16N, H16R, H16S, H16T, H16V, H16Y, L19A, L19D, L19E, L19G, L19N, L19R, L19S, L19T, L19V, D20A, D20E, D20H, D201, D20Y, D20F, D20G, D20T, D20W, M23R, V69A, Q47P, R81A, R81G, R81S, R81T, D84A, D84E, D84G, D84K, D841, D84M, D84Q D84R, D84S, D84T, S87R, N88A, N88D, N88R, N88E, N88I, N88F, N88G, N88M, N88R, N88S, N88V, N88W, V91D, V91E, V91K, V91G, V91N, V91R, V91S, 192K, I92R, E95G, C125A, C125S, Q1261, Q126L, and Q126F. It is understood that any mutation can be combined with any other mutation in any order from the above list. It is understood that any number of mutations from the above list can be combined in any order. It is understood that every position described above may be mutated and replaced by an amino acid substitution as described above or an equivalent amino acid.

In some embodiments of the bifunctional fusion protein, the IL-2 polypeptide comprises at least one amino acid substitution selected from N88R, N88S, C125S, V69A and Q74P. In some embodiments, the IL-2 polypeptide comprises an N88R amino acid substitution. In some embodiments, the IL-2 polypeptide comprises an N88S amino acid substitution. In some embodiments, the IL-2 polypeptide comprises an C125S amino acid substitution. In some embodiments, the IL-2 polypeptide comprises an V69A amino acid substitution. In some embodiments, the IL-2 polypeptide comprises an Q74P amino acid substitution.

In some embodiments, the IL-2 polypeptide comprises 2, 3, 4, or 5 mutations selected from N88R, N88S, C125S, V69A and Q74P. In some embodiments, the IL-2 polypeptide comprises N88R and C125S mutations. In some embodiments, the IL-2 polypeptide comprises N88R and V69A mutations. In some embodiments, the IL-2 polypeptide comprises N88R and Q74P mutations. In some embodiments, the IL-2 polypeptide comprises N88S and C125S mutations. In some embodiments, the IL-2 polypeptide comprises N88S and V69A mutations. In some embodiments, the IL-2 polypeptide comprises N88S and Q74P mutations. In some embodiments, the IL-2 polypeptide comprises C125S and V69A mutations. In some embodiments, the IL-2 polypeptide comprises C125S and Q74P mutations. In some embodiments, the IL-2 polypeptide comprises V69A and Q74P mutations.

In some embodiments, the IL-2 polypeptide comprises N88R, C125S and Q74P mutations. In some embodiments, the IL-2 polypeptide comprises N88S, C125S and Q74P mutations. In some embodiments, the IL-2 polypeptide comprises N88R, V69A and Q74P mutations. In some embodiments, the IL-2 polypeptide comprises N88S, V69A and Q74P mutations. In some embodiments, the IL-2 polypeptide comprises N88R, C125S and V69A mutations. In some embodiments, the IL-2 polypeptide comprises N88S, C125S and V69A mutations.

In some embodiments, the IL-2 variant comprises the amino acid substitution N88R. In some embodiments, the IL-2 variant comprises the amino acid substitution N88S.

In some embodiments, the IL-2 variant comprises the amino acid substitutions of N88D and C125S.

In some embodiments, the IL-2 variant comprises the amino acid substitutions of V69A, Q47P, N88D and C125S.

In some embodiments, a suitable recombinant IL-2 variant has an in vivo half-life of or greater than about 1 minute, 2 minutes, 10 minutes, 15 minutes, 30 minutes, 60 minutes, 70 minutes, 80 minutes, 90 minutes, 100 minutes, 2 hours, 3 hours, 4 hours, 5 hours, 6 hours, 7 hours, 8 hours, 9 hours, 10 hours, 12 hours, or 24 hours. In some embodiments, a suitable recombinant IL-2 mutein or a recombinant IL-2 fusion protein has an in vivo half-life of or greater than about 24 hours, 30 hours, 36 hours, 42 hours, 48 hours, 54 hours, or 60 hours. In some embodiments, a recombinant IL-2 mutein has an in vivo half-life of between 0.5 and 24 hours, between 1 day and 10 days, between 1 day and 9 days, between 1 day and 8 days, between 1 day and 7 days, between 1 day and 6 days, or between 1 day and 5 days.

The sequences of wild-type IL-2 and exemplary IL-2 variants are provided in Table 2.

### Interleukin-33 (IL-33) Variants

In some embodiments of the bifunctional protein, the interleukin-33 (IL-33) polypeptide is a variant or mutein comprising one or more mutations relative to a wild-type IL-33 polypeptide. IL-33 is a pleiotropic cytokine, also referred to as “alarmin”, which is released upon tissue injury. Tissue-derived immune cells, including Tregs, innate lymphoid cells (ILC2s), mast cells constitutively express the IL-33 receptor, also known as the ST2. IL-33 also activates additional immune cells, such as, M2 macrophages, and dendritic cells. In addition, immune cells including NK cells, iNKT cells and neutrophils, in some embodiments, constitutively express ST2. In some embodiments, ST2 expression is inducible and regulated by the tissue microenvironment.

Human IL-33 is a 270 amino acid protein belonging to the IL-1 cytokine family, and is composed of two evolutionary conserved domains, the N-terminal nuclear domain and the C-terminal IL-1-like cytokine domain, separated by a divergent ‘protease sensor’ domain. The N-terminal domain (aa 1-65) is required for nuclear localization and comprises a chromatin-binding motif (CBM). The ‘protease sensor’ domain of IL-33 (aa 66 to 111) is a protease cleavage and activation site.

The C-terminal domain (cytokine domain, aa 112-270) possesses cytokine activity and has a three-dimensional structure similar to interleukin 1 (IL-1). The C-terminal is responsible for binding to the ST2 receptor along with a co-receptor IL-1 receptor accessory protein (IL-1RAcP) that facilitates the interaction. The IL-33-ST2 interaction is mediated by surface charge complementarity. Protease cleavage of IL-33 yields an 18 kDa C-terminal fragment, which is referred to herein as truncated IL-33. Truncated IL-33 by itself can stimulate Treg cells, ILCs, Th2 cells, macrophages, and dendritic cells, and thereby upregulate the expression of IL-2, IL-4, IL-5, and IL-13, and also reduce pro-inflammatory Th1 and Th17 responses. Fusion proteins comprising truncated WT IL-33 domains have been described in the art, for example, fusions with IL-2, U.S. Pat. No. 9,840,545B2; U.S. Pat. No. 10,851,145B2; and US 20210261640A1; the disclosures of which are hereby incorporated by reference.

The present invention provides, among other things, IL-33 variants or IL-33 muteins having improved properties including manufacturability and/or activity.

In some aspects, provided herein is a recombinant human interleukin-33 (IL-33) variant comprising at least one amino acid substitution at N60, C97, C116, C121, or C148, wherein the substitution at C97, C116, C121, or C148 is not a serine and wherein the IL-33 has improved activity and/or manufacturability relative to wild-type IL-33. As used herein, wild-type IL-33 used for comparison is a truncated IL-33 lacking site-specific mutations and the amino acid positions noted here are relative to truncated IL-33. In some embodiments, provided herein is a recombinant human interleukin-33 (IL-33) variant comprising an amino acid substitution at C97, wherein the substitution is not a serine and wherein the IL-33 has improved activity and/or manufacturability relative to wild-type IL-33. In some embodiments, provided herein is a recombinant human interleukin-33 (IL-33) variant comprising an amino acid substitution at C116, wherein the substitution is not a serine and wherein the IL-33 has improved activity and/or manufacturability relative to wild-type IL-33. In some embodiments, provided herein is a recombinant human interleukin-33 (IL-33) variant comprising an amino acid substitution at C121, wherein the substitution is not a serine and wherein the IL-33 has improved activity and/or manufacturability relative to wild-type IL-33. In some embodiments, provided herein is a recombinant human interleukin-33 (IL-33) variant comprising an amino acid substitution at C148, wherein the substitution is not a serine and wherein the IL-33 has improved activity and/or manufacturability relative to wild-type IL-33. In some embodiments, provided herein is a recombinant human interleukin-33 (IL-33) variant comprising an amino acid substitution at N60, and wherein the IL-33 has improved activity and/or manufacturability relative to wild-type IL-33.

In some aspects, provided herein is a recombinant human interleukin-33 (IL-33) variant comprising an amino acid substitution at N60 and any one of C97, C116, C121, or C148. In some embodiments, provided herein is a recombinant human interleukin-33 (IL-33) variant comprising an amino acid substitution at N60 and C97. In some embodiments, provided herein is a recombinant human interleukin-33 (IL-33) variant comprising an amino acid substitution at N60 and C116. In some embodiments, provided herein is a recombinant human interleukin-33 (IL-33) variant comprising an amino acid substitution at N60 and C121. In some embodiments, provided herein is a recombinant human interleukin-33 (IL-33) variant comprising an amino acid substitution at N60 and C148.

In some aspects, provided herein is a recombinant human interleukin-33 (IL-33) variant comprising an amino acid substitution C116F and an additional amino acid substitution at any one of C97, C121, or C148. In some embodiments, provided herein is a recombinant human interleukin-33 (IL-33) variant comprising an amino acid substitution C116F and an additional amino acid substitution at C97. In some embodiments, provided herein is a recombinant human interleukin-33 (IL-33) variant comprising an amino acid substitution C116F and an additional amino acid substitution at C121. In some embodiments, provided herein is a recombinant human interleukin-33 (IL-33) variant comprising an amino acid substitution C116F and an additional amino acid substitution at C148.

In some aspects, provided herein is a recombinant human interleukin-33 (IL-33) variant comprising at least one amino acid substitution selected from N60S, N60D, C97G, and C116F. In some embodiments, provided herein is a recombinant human interleukin-33 (IL-33) variant comprising an N60S amino acid substitution. In some embodiments, provided herein is a recombinant human interleukin-33 (IL-33) variant comprising an N60D amino acid substitution. In some embodiments, provided herein is a recombinant human interleukin-33 (IL-33) variant comprising an C97G amino acid substitution. In some embodiments, provided herein is a recombinant human interleukin-33 (IL-33) variant comprising an C116F amino acid substitution. In some embodiments, provided herein is a recombinant human interleukin-33 (IL-33) variant comprising an N60S and a C97G amino acid substitution. In some embodiments, provided herein is a recombinant human interleukin-33 (IL-33) variant comprising an N60D and a C97G amino acid substitution. In some embodiments, provided herein is a recombinant human interleukin-33 (IL-33) variant comprising an N60S and a C116F amino acid substitution. In some embodiments, provided herein is a recombinant human interleukin-33 (IL-33) variant comprising an N60D and a C116F amino acid substitution. In some embodiments, provided herein is a recombinant human interleukin-33 (IL-33) variant comprising an N60S, C97G and a C116F amino acid substitution. In some embodiments, provided herein is a recombinant human interleukin-33 (IL-33) variant comprising an N60D, C97G and a C116F amino acid substitution.

In some embodiments, the recombinant human IL-33 variant comprises the amino acid substitutions of C97G and C116F.

In some embodiments, the recombinant human IL-33 variant is a truncated IL-33.

In some embodiments, the recombinant human IL-33 variant polypeptide comprises the sequence of any one of SEQ ID NO: 22 to SEQ ID NO:28.

In some embodiments, IL-33 variants described herein are associated with a second therapeutic agent. In some embodiments, the second therapeutic agent is a cytokine. In some embodiments, IL-33 variants are associated with an IL-2 polypeptide. In some embodiments, IL-33 variants are fused with an IL-2 polypeptide in a bifunctional fusion protein.

The sequences of wild-type human IL-33 and exemplary IL-33 variants are provided in Table 3.

### Bifunctional IL-33 and IL-2 Fusion Protein

In some aspects, an IL-2 polypeptide is associated with an IL-33 variant in a bifunctional protein that synergistically facilitates a therapeutic effect, for example, by enhancing or increasing stability, potency and/or delivery of IL-2 protein, reducing or eliminating immunogenicity, or clearance, etc. The present invention provides bifunctional proteins comprising variants that preferentially expand Tregs over, for example, T effector or NK cells. In some embodiments, the bifunctional protein described herein is a fusion protein between an IL-2 domain and an IL-33 variant.

In some aspects, provided herein is a fusion protein comprising, an IL-2 polypeptide, and an IL-33 polypeptide comprising at least one amino acid substitution at positions selected from N60, C97, C116, C121, and C148 according to the sequence of SEQ ID NO. 21, wherein the IL-2 polypeptide is linked to the IL-33 polypeptide via a linker.

In some aspects, provided herein is a fusion protein comprising, an IL-2 polypeptide, and an IL-33 polypeptide comprising amino acid substitutions of N60S, C97G, and C116F, according to the sequence of SEQ ID NO: 21, wherein the IL-2 polypeptide is linked to the IL-33 polypeptide via a linker.

In some embodiments of the fusion protein, the IL-33 polypeptide further comprises an amino acid substitution C148G.

In some embodiments of the fusion protein, the IL-33 polypeptide further comprises an amino acid substitution C121S.

In some aspects, provided herein is a fusion protein comprising, an IL-2 polypeptide, and an IL-33 polypeptide comprising at least one amino acid substitution at position C97 according to the sequence of SEQ ID NO. 21, wherein the IL-2 polypeptide is linked to the IL-33 polypeptide via a linker. In some aspects, provided herein is a fusion protein comprising, an IL-2 polypeptide, and an IL-33 polypeptide comprising at least one amino acid substitution at position C116 according to the sequence of SEQ ID NO. 21, wherein the IL-2 polypeptide is linked to the IL-33 polypeptide via a linker. In some aspects, provided herein is a fusion protein comprising, an IL-2 polypeptide, and an IL-33 polypeptide comprising at least one amino acid substitution at position C121 according to the sequence of SEQ ID NO. 21, wherein the IL-2 polypeptide is linked to the IL-33 polypeptide via a linker. In some aspects, provided herein is a fusion protein comprising, an IL-2 polypeptide, and an IL-33 polypeptide comprising at least one amino acid substitution at position C148 according to the sequence of SEQ ID NO. 21, wherein the IL-2 polypeptide is linked to the IL-33 polypeptide via a linker.

In some aspects, provided herein is a fusion protein comprising, an IL-2 polypeptide, and an IL-33 polypeptide comprising at least one amino acid substitution at positions selected from N60, C97, C116, C121, and C148 according to the sequence of SEQ ID NO: 21, wherein the substitution at C97, C116, C121, or C148 is not a serine, wherein the IL-2 polypeptide is linked to the IL-33 polypeptide via a linker.

In some aspects, provided herein is a fusion protein comprising, an IL-2 polypeptide, and an IL-33 polypeptide comprising at least one amino acid substitution at position N60, wherein the IL-2 polypeptide is linked to the IL-33 polypeptide via a linker. In some aspects, provided herein is a fusion protein comprising, an IL-2 polypeptide, and an IL-33 polypeptide comprising at least one amino acid substitution at position C97 according to the sequence of SEQ ID NO. 21, wherein the substitution at C97 is not a serine, wherein the IL-2 polypeptide is linked to the IL-33 polypeptide via a linker. In some aspects, provided herein is a fusion protein comprising, an IL-2 polypeptide, and an IL-33 polypeptide comprising at least one amino acid substitution at position C116 according to the sequence of SEQ ID NO. 21, wherein the substitution at C116 is not a serine, wherein the IL-2 polypeptide is linked to the IL-33 polypeptide via a linker. In some aspects, provided herein is a fusion protein comprising, an IL-2 polypeptide, and an IL-33 polypeptide comprising at least one amino acid substitution at position C121 according to the sequence of SEQ ID NO. 21, wherein the substitution at C121 is not a serine, wherein the IL-2 polypeptide is linked to the IL-33 polypeptide via a linker.

In some aspects, provided herein is a fusion protein comprising, an IL-2 polypeptide, and an IL-33 polypeptide comprising at least one amino acid substitution at position C148 according to the sequence of SEQ ID NO. 21, wherein the substitution at C148 is not a serine, wherein the IL-2 polypeptide is linked to the IL-33 polypeptide via a linker.

In some aspects, provided herein is a fusion protein comprising, an IL-2 polypeptide, and an IL-33 polypeptide comprising the amino acid substitution C116F, C116Y or C116A according to the sequence of SEQ ID NO: 21, wherein the IL-2 polypeptide is linked to the IL-33 polypeptide via a linker. In some aspects, provided herein is a fusion protein comprising, an IL-2 polypeptide, and an IL-33 polypeptide comprising the amino acid substitution C116F according to the sequence of SEQ ID NO: 21, wherein the IL-2 polypeptide is linked to the IL-33 polypeptide via a linker. In some aspects, provided herein is a fusion protein comprising, an IL-2 polypeptide, and an IL-33 polypeptide comprising the amino acid substitution C116Y according to the sequence of SEQ ID NO: 21, wherein the IL-2 polypeptide is linked to the IL-33 polypeptide via a linker. In some aspects, provided herein is a fusion protein comprising, an IL-2 polypeptide, and an IL-33 polypeptide comprising the amino acid substitution C116A according to the sequence of SEQ ID NO: 21, wherein the IL-2 polypeptide is linked to the IL-33 polypeptide via a linker.

In some embodiments of the fusion protein, the IL-33 polypeptide further comprises amino acid substitution at positions N60, C97, C121 and/or C148.

In some embodiments of the fusion protein, the IL-33 polypeptide further comprises at least one amino acid substitution at positions selected from N60, C97, C121 and C148. In some embodiments, the IL-33 polypeptide further comprises at least one amino acid substitution at N60. In some embodiments of the fusion protein, the IL-33 polypeptide further comprises at least one amino acid substitution at C97. In some embodiments of the fusion protein, the IL-33 polypeptide further comprises at least one amino acid substitution at C121. In some embodiments of the fusion protein, the IL-33 polypeptide further comprises at least one amino acid substitution at C148. In some embodiments, the IL-33 polypeptide comprises one or more amino acid substitutions N60S, N60D, C97G, C116F, C121S or C148G. In some embodiments, the IL-33 polypeptide comprises the amino acid substitutions N60S, C97G, and C116F. In some embodiments, the IL-33 polypeptide further comprises the amino acid substitution C148G. In some embodiments, the IL-33 polypeptide further comprises the amino acid substitution C121S.

In some embodiments of the fusion protein, the IL-33 polypeptide comprises one amino acid substitution of N60S, N60D, C97G, or C116F. In some embodiments of the fusion protein, the IL-33 polypeptide comprises one amino acid substitution of N60S. In some embodiments of the fusion protein, the IL-33 polypeptide comprises one amino acid substitution of N60D. In some embodiments of the fusion protein, the IL-33 polypeptide comprises one amino acid substitution of C97G. In some embodiments of the fusion protein, the IL-33 polypeptide comprises one amino acid substitution of C116F.

In some embodiments of the fusion protein, the IL-33 polypeptide comprises two amino acid substitutions selected from N60S, C97G, and C116F. In some embodiments, the IL-33 polypeptide comprises two amino acid substitutions of N60S and C97G. In some embodiments, the IL-33 polypeptide comprises two amino acid substitutions of N60S and C116F. In some embodiments, the IL-33 polypeptide comprises two amino acid substitutions of C97G and C116F.

In some embodiments of the fusion protein, the IL-33 polypeptide comprises three amino acid substitutions of N60S, C97G, and C116F.

In some embodiments of the fusion protein, the IL-33 polypeptide further comprises two substitutions N60S and C97G.

In some embodiments of the fusion protein, the IL-2 polypeptide is an IL-2 variant biased to IL2Rα.

In some embodiments of the fusion protein, the IL-2 variant comprises one or more amino acid substitutions selected from T3N, T3A, L12G, L12K, L12Q, L12S, Q13G, E15A, E15G, E15S, H16A, H16D, H16E, H16G, H16K, H16M, H16N, H16R, H16S, H16T, H16V, H16Y, L19A, L19D, L19E, L19G, L19N, L19R, L19S, L19T, L19V, D20A, D20E, D20H, D201, D20Y, D20F, D20G, D20T, D20W, M23R, V69A, Q47P, R81A, R81G, R81S, R81T, D84A, D84E, D84G, D84K, D841, D84M, D84Q D84R, D84S, D84T, S87R, N88A, N88D, N88R, N88E, N88I, N88F, N88G, N88M, N88R, N88S, N88V, N88W, V91D, V91E, V91K, V91G, V91N, V91R, V91S, 192K, I92R, E95G, C125A, C125S Q1261, Q126L, and Q126F.

In some embodiments of the fusion protein, the IL-2 variant comprises one or more amino acid substitutions selected from V69A, Q47P, N88D and C125S. In some embodiments, the IL-2 variant comprises an amino acid substitution at V69A. In some embodiments, the IL-2 variant comprises an amino acid substitution at Q74P. In some embodiments, the IL-2 variant comprises an amino acid substitution at N88D. In some embodiments, the IL-2 variant comprises an amino acid substitution at C125S. In some embodiments, the IL-2 variant comprises two amino acid substitutions selected from V69A, Q47P, N88D and C125S, in any order and in any combination. In some embodiments, the IL-2 variant comprises three amino acid substitutions selected from V69A, Q47P, N88D and C125S, in any order and in any combination. In some embodiments, the IL-2 variant comprises three amino acid substitutions at Q47P, N88D and C125S. In some embodiments, the IL-2 variant comprises three amino acid substitutions at V69A, N88D and C125S. In some embodiments, the IL-2 variant comprises three amino acid substitutions at V69A, Q47P and C125S. In some embodiments, the IL-2 variant comprises three amino acid substitutions at V69A, Q47P and N88D. In some embodiments, the IL-2 variant comprises four amino acid substitutions at V69A, Q47P, N88D and C125S.

In some embodiments of the bifunctional protein or the fusion protein, the IL-2 variant or polypeptide comprises an amino acid selected from SEQ ID NO: 3 to SEQ ID NO: 20, and wherein the IL-33 variant or polypeptide comprises an amino acid sequence selected from SEQ ID NO: 22 to SEQ ID NO: 28.

In some embodiments of the bifunctional protein or the fusion protein, wherein the peptide linker is selected from GGGGS (SEQ ID NO: 29; n repeats of SEQ ID NO: 29, n=1-5), GGGGSGGGGSGGGGS (SEQ ID NO: 31), EAAAK (SEQ ID NO: 32; n repeats of SEQ ID NO: 32, n=1-3), GGGGGGGG (SEQ ID NO: 33), GSGSGSGSGS (SEQ ID NO: 34), GSGSGSGSGSGSGSGSGSGS (SEQ ID NO: 35), GGSGGSGGS (SEQ ID NO: 36), GSGGS (SEQ ID NO: 37), GSSGS (SEQ ID NO: 38), a SEG-linker, a GSAT (SEQ ID NO: 39), SSSSGSSSSG (SEQ ID NO: 40), a flexible 22 amino acid linker IEGSGQGPGSGQGSGSPGSGQG (SEQ ID NO. 41), and GGGGSEAAAK (SEQ ID NO: 42, n=1-2), EGKSSGSGSESKST (SEQ ID NO: 44), GGGGSLVPRGSGGGGS (SEQ ID NO: 45), KESGSVSSEQLAQFRSLD (SEQ ID NO: 46), GGGSEGGGSEGGGSEGGG (SEQ ID NO: 47) or rigid peptide linkers PAPAP (SEQ ID NO: 48), (Ala-Pro)ₙ, AEAAAKEAAAKA (SEQ ID NO: 49), AEAAAKEAAAKEAAAKEAAAKALEAEAAAKEAAAKEAAAKEAAAKA (SEQ ID NO: 50).

In some embodiments of the bifunctional protein or the fusion protein, wherein the linker comprises GGGGSGGGGSGGGGS (SEQ ID NO: 31).

In some embodiments of the bifunctional protein or the fusion protein, wherein the protein further comprises a signal peptide.

In some embodiments of the bifunctional protein or the fusion protein, wherein the IL-33 variant or polypeptide has increased activity and/or manufacturability as compared to human WT IL-33.

In some embodiments of a bifunctional protein comprising an IL-2 variant associated with an IL-33 variant, wherein the IL-2 variant has amino acid substitution N88D and the IL-33 variant has amino acid substitution C116F.

In some embodiments of the bifunctional protein, wherein the IL-2 variant has amino acid substitutions N88D and C125S and the IL-33 variant has amino acid substitutions C97G, C116F, C121S and/or C148G.

In some embodiments of the bifunctional protein, wherein the IL-2 variant is linked to the IL-33 variant by a linker.

In some embodiments of the bifunctional protein, wherein the linker is a peptide linker or a chemical linker. In some embodiments of the bifunctional protein, wherein the linker is a peptide linker. In some embodiments of the bifunctional protein, wherein the linker is a chemical linker.

In some embodiments of the bifunctional protein, wherein the bifunctional protein, upon administration in vivo, results in eosinophil level at less than 5-fold, less than 4 fold, less than 3 fold or less than 2 fold as compared to the level before the treatment. In some embodiments of the bifunctional protein, wherein the bifunctional protein, upon administration in vivo, results in eosinophil level at less than 5-fold as compared to the level before the treatment. In some embodiments of the bifunctional protein, wherein the bifunctional protein, upon administration in vivo, results in eosinophil levels at less than 4 fold as compared to the level before the treatment. In some embodiments of the bifunctional protein, wherein the bifunctional protein, upon administration in vivo, results in eosinophil levels at less than 3 fold as compared to the level before the treatment. In some embodiments of the bifunctional protein, wherein the bifunctional protein, upon administration in vivo, results in eosinophil levels at less than 2 fold as compared to the level before the treatment.

In some aspects, provided herein is fusion protein comprising an amino acid sequence at least 85%, 90%, 95%, 96%, 97%, 98%, or 100% identical to any one of the sequences of SEQ ID NO: 54-75 and SEQ ID NO: 80-81. In some aspects, provided herein is fusion protein comprising an amino acid sequence at least 85% identical to any one of the sequences of SEQ ID NO: 54-75 and SEQ ID NO: 80-81. In some aspects, provided herein is fusion protein comprising an amino acid sequence at least 90% identical to any one of the sequences of SEQ ID NO: 54-75 and SEQ ID NO: 80-81. In some aspects, provided herein is fusion protein comprising an amino acid sequence at least 95% identical to any one of the sequences of SEQ ID NO: 54-75 and SEQ ID NO: 80-81. In some aspects, provided herein is fusion protein comprising an amino acid sequence at least 96% identical to any one of the sequences of SEQ ID NO: 54-75 and SEQ ID NO: 80-81. In some aspects, provided herein is fusion protein comprising an amino acid sequence at least 97% identical to any one of the sequences of SEQ ID NO: 54-75 and SEQ ID NO: 80-81. In some aspects, provided herein is fusion protein comprising an amino acid sequence at least 98% identical to any one of the sequences of SEQ ID NO: 54-75 and SEQ ID NO: 80-81. In some aspects, provided herein is fusion protein comprising an amino acid sequence at least 100% identical to any one of the sequences of SEQ ID NO: 54-75 and SEQ ID NO: 80-81.

In some aspects, provided herein is a bifunctional fusion protein comprising an amino acid sequence having at least 90% identity to SEQ ID NO: 57. In some embodiments, the bifunctional fusion protein comprises an amino acid sequence having at least 95% identity to SEQ ID NO: 57. In some embodiments, the bifunctional fusion protein comprises an amino acid sequence having 100% identity to SEQ ID NO: 57.

In some aspects, provided herein is a bifunctional fusion protein comprising an amino acid sequence having at least 90% identity to SEQ ID NO: 61. In some embodiments, the bifunctional fusion protein comprises an amino acid sequence having at least 95% identity to SEQ ID NO: 61. In some embodiments, the bifunctional fusion protein comprises an amino acid sequence having 100% identity to SEQ ID NO: 61.

In some aspects, provided herein is a bifunctional fusion protein comprising an amino acid sequence having at least 90% identity to SEQ ID NO: 69. In some embodiments, the bifunctional fusion protein comprises an amino acid sequence having at least 95% identity to SEQ ID NO: 69. In some embodiments, the bifunctional fusion protein comprises an amino acid sequence having 100% identity to SEQ ID NO: 69.

In some aspects, provided herein is a bifunctional fusion protein comprising an amino acid sequence having at least 90% identity to SEQ ID NO: 70. In some embodiments, the bifunctional fusion protein comprises an amino acid sequence having at least 95% identity to SEQ ID NO: 70. In some embodiments, the bifunctional fusion protein comprises an amino acid sequence having 100% identity to SEQ ID NO: 70.

In some aspects, provided herein is a bifunctional fusion protein comprising an amino acid sequence having at least 90% identity to SEQ ID NO: 80. In some aspects, provided herein is a bifunctional fusion protein comprising an amino acid sequence having at least 95% identity to SEQ ID NO: 80. In some aspects, provided herein is a bifunctional fusion protein comprising an amino acid sequence having 100% identity to SEQ ID NO: 80.

In some aspects, provided herein is a bifunctional fusion protein comprising an amino acid sequence having at least 90% identity to SEQ ID NO: 81. In some aspects, provided herein is a bifunctional fusion protein comprising an amino acid sequence having at least 95% identity to SEQ ID NO: 81. In some aspects, provided herein is a bifunctional fusion protein comprising an amino acid sequence having 100% identity to SEQ ID NO: 81.

In some aspects, provided herein is a bifunctional fusion protein, wherein the IL-33 polypeptide is a variant wherein the variant has improved activity and/or manufacturability relative to wild-type IL-33. In some aspects, provided herein is a bifunctional fusion protein, wherein the IL-33 polypeptide is a variant wherein the variant has improved activity relative to wild-type IL-33. In some aspects, provided herein is a bifunctional fusion protein, wherein the IL-33 polypeptide is a variant wherein the variant has improved manufacturability relative to wild-type IL-33.

In some aspects, provided herein is a bifunctional fusion protein, comprising at least one amino acid substitution at N60, C97, C116, C121, or C148. In some embodiments, provided herein is a bifunctional fusion protein, comprising at least one amino acid substitution at N60. In some embodiments, provided herein is a bifunctional fusion protein, comprising at least one amino acid substitution at C97. In some embodiments, provided herein is a bifunctional fusion protein, comprising at least one amino acid substitution at C116. In some embodiments, provided herein is a bifunctional fusion protein, comprising at least one amino acid substitution at C121. In some embodiments, provided herein is a bifunctional fusion protein, comprising at least one amino acid substitution at C148.

In some aspects, provided herein is a bifunctional fusion protein, comprising at least one amino acid substitution at N60, C97, C116, C121, or C148, and wherein the substitution at any one of the cysteines C97, C116, C121, or C148 is not a serine.

In some embodiments, provided herein is a bifunctional fusion protein, comprising at least one amino acid substitution at N60. In some embodiments, provided herein is a bifunctional fusion protein, comprising at least one amino acid substitution at C97, wherein the substitution at C97 is not a serine. In some embodiments, provided herein is a bifunctional fusion protein, comprising at least one amino acid substitution at C116, wherein the substitution at C116 is not a serine. In some embodiments, provided herein is a bifunctional fusion protein, comprising at least one amino acid substitution at C121, wherein the substitution at C121 is not a serine. In some embodiments, provided herein is a bifunctional fusion protein, comprising at least one amino acid substitution at C148, wherein the substitution at C148 is not a serine.

In some embodiments, provided herein is a bifunctional fusion protein, comprising amino acid substitutions at N60 and C97, and wherein the substitution at C97 is not a serine. In some embodiments, provided herein is a bifunctional fusion protein, comprising amino acid substitutions at N60 and C116, and wherein the substitution at C116 is not a serine. In some embodiments, provided herein is a bifunctional fusion protein, comprising amino acid substitutions at N60 and C121, and wherein the substitution at C121 is not a serine. In some embodiments, provided herein is a bifunctional fusion protein, comprising amino acid substitutions at N60 and C148, and wherein the substitution at C148 is not a serine. In some embodiments, provided herein is a bifunctional fusion protein, comprising amino acid substitutions at C97 and C116, and wherein the substitution at C97 and C116 is not a serine. In some embodiments, provided herein is a bifunctional fusion protein, comprising amino acid substitutions at C97 and C121, and wherein the substitution at C97 and C121 is not a serine. In some embodiments, provided herein is a bifunctional fusion protein, comprising amino acid substitutions at C97 and C148, and wherein the substitution at C97 and C148 is not a serine. In some embodiments, provided herein is a bifunctional fusion protein, comprising amino acid substitutions at C116 and C121, and wherein the substitution at C116 and C121 is not a serine. In some embodiments, provided herein is a bifunctional fusion protein, comprising amino acid substitutions at C116 and C148, and wherein the substitution at C116 and C148 is not a serine. In some embodiments, provided herein is a bifunctional fusion protein, comprising amino acid substitutions at C121 and C148, and wherein the substitution at C121 and C148 is not a serine. In some embodiments, provided herein is a bifunctional fusion protein, comprising amino acid substitutions at C97 and C121, and wherein the substitution at C97 and C121 is not a serine.

In some embodiments, provided herein is a bifunctional fusion protein, comprising amino acid substitutions at C97, C116 and C121, and wherein the substitution at C97, C116 and C121 is not a serine. In some embodiments, provided herein is a bifunctional fusion protein, comprising amino acid substitutions at C97, C121 and C148, and wherein the substitution at C97, C121 and C148 is not a serine. In some embodiments, provided herein is a bifunctional fusion protein, comprising amino acid substitutions at C97, C116 and C148, and wherein the substitution at C97, C116 and C148 is not a serine. In some embodiments, provided herein is a bifunctional fusion protein, comprising amino acid substitutions at C116, C121 and C148, and wherein the substitution at C116, C121 and C148 is not a serine.

In some embodiments, the bifunctional fusion protein comprises an amino acid substitution at N60 and any one of C97, C116, C121, or C148. In some embodiments, the bifunctional fusion protein comprises amino acid substitutions at N60 and C97. In some embodiments, the bifunctional fusion protein comprises amino acid substitutions at N60 and C116. In some embodiments, the bifunctional fusion protein comprises amino acid substitutions at N60 and C121. In some embodiments, the bifunctional fusion protein comprises amino acid substitutions at N60 and C148.

In some embodiments, the bifunctional fusion protein comprises an amino acid substitution at C116F and an additional amino acid substitution at any one of C97, C121, or C148. In some embodiments, the bifunctional fusion protein comprises an amino acid substitution at C116F and an additional amino acid substitution at C97. In some embodiments, the bifunctional fusion protein comprises an amino acid substitution at C116F and an additional amino acid substitution at C121. In some embodiments, the bifunctional fusion protein comprises an amino acid substitution at C116F and an additional amino acid substitution at C148.

In some embodiments, the bifunctional fusion protein comprises at least one amino acid substitution selected from N60S, N60D, C97G, and C116F. In some embodiments, the bifunctional fusion protein comprises an amino acid substitution N60S. In some embodiments, the bifunctional fusion protein comprises an amino acid substitution N60D. In some embodiments, the bifunctional fusion protein comprises an amino acid substitution C97G. In some embodiments, the bifunctional fusion protein comprises an amino acid substitution C116F.

In some embodiments, the bifunctional fusion protein comprises the amino acid substitutions of C97G and C116F.

In some embodiments of the bifunctional fusion protein, the IL-33 is truncated.

In some embodiments of the bifunctional fusion protein, the IL-33 polypeptide comprises the sequence of any one of SEQ ID NO: 22-28.

In some aspects, provided herein is a bifunctional protein comprising: an IL-2 polypeptide, and an IL-33 variant that has improved activity and/or manufacturability as compared to human WT IL-33, wherein the IL-2 polypeptide is associated with the IL-33 variant, and wherein the bifunctional protein preferentially expands ST2+ Treg cells.

The sequences of exemplary bifunctional proteins are provided at SEQ ID NOs. 54 to SEQ ID NO:75 and SEQ ID NO: 80 to SEQ ID NO: 81, in Table 5.

In some embodiments of the bifunctional fusion protein, wherein the one or more mutations are selected from N297A, N297Q, N297G, L234A, L235E, G237A, P238S, H268Q, H268A, V309L, A330S and P331S. In some embodiments, the one or more mutations are N297A. In some embodiments, the mutation is N297Q. In some embodiments, the mutation is N297G. In some embodiments, the mutation is L234A. In some embodiments, the mutation is L235E. In some embodiments, the mutation is G237A. In some embodiments, the mutation is P238S. In some embodiments, the mutation is H268Q. In some embodiments, the mutation is H268A. In some embodiments, the mutation is V309L. In some embodiments, the mutation is A330S. In some embodiments, the mutation is P331S.

In some embodiments, the bifunctional fusion protein comprises two mutations selected from N297A, N297Q, N297G, L234A, L235E, G237A, P238S, H268Q, H268A, V309L, A330S and P331S. It is to be understood by a skilled person that this includes any and all permutations and combinations of two mutations from the above list selected in any order.

In some embodiments, the bifunctional fusion protein comprises 3 mutations selected from N297A, N297Q, N297G, L234A, L235E, G237A, P238S, H268Q, H268A, V309L, A330S and P331S. It is to be understood by a skilled person that this includes any and all permutations and combinations of 3 mutations from the above list selected in any order.

In some embodiments, the bifunctional fusion protein comprises 4 mutations selected from N297A, N297Q, N297G, L234A, L235E, G237A, P238S, H268Q, H268A, V309L, A330S and P331S. It is to be understood by a skilled person that this includes any and all permutations and combinations of 4 mutations from the above list selected in any order.

In some embodiments, the bifunctional fusion protein comprises 5 mutations selected from N297A, N297Q, N297G, L234A, L235E, G237A, P238S, H268Q, H268A, V309L, A330S and P331S. It is to be understood by a skilled person that this includes any and all permutations and combinations of 5 mutations from the above list selected in any order.

In some embodiments, the bifunctional fusion protein comprises 6 mutations selected from N297A, N297Q, N297G, L234A, L235E, G237A, P238S, H268Q, H268A, V309L, A330S and P331S. It is to be understood by a skilled person that this includes any and all permutations and combinations of 6 mutations from the above list selected in any order.

In some embodiments, the bifunctional fusion protein comprises 7 mutations selected from N297A, N297Q, N297G, L234A, L235E, G237A, P238S, H268Q, H268A, V309L, A330S and P331S. It is to be understood by a skilled person that this includes any and all permutations and combinations of 7 mutations from the above list selected in any order.

In some embodiments, the bifunctional fusion protein comprises 8 mutations selected from N297A, N297Q, N297G, L234A, L235E, G237A, P238S, H268Q, H268A, V309L, A330S and P331S. It is to be understood by a skilled person that this includes any and all permutations and combinations of 8 mutations from the above list selected in any order.

In some embodiments, the bifunctional fusion protein comprises 9 mutations selected from N297A, N297Q, N297G, L234A, L235E, G237A, P238S, H268Q, H268A, V309L, A330S and P331S. It is to be understood by a skilled person that this includes any and all permutations and combinations of 9 mutations from the above list selected in any order.

In some embodiments, the bifunctional fusion protein comprises 10 mutations selected from N297A, N297Q, N297G, L234A, L235E, G237A, P238S, H268Q, H268A, V309L, A330S and P331S. It is to be understood by a skilled person that this includes any and all permutations and combinations of 10 mutations from the above list selected in any order.

In some embodiments, the bifunctional fusion protein comprises 11 mutations selected from N297A, N297Q, N297G, L234A, L235E, G237A, P238S, H268Q, H268A, V309L, A330S and P331S. It is to be understood by a skilled person that this includes any and all permutations and combinations of 11 mutations from the above list selected in any order. In some embodiments, the bifunctional fusion protein comprises 11 mutations selected from N297Q, N297G, L234A, L235E, G237A, P238S, H268Q, H268A, V309L, A330S and P331S. In some embodiments, the bifunctional fusion protein comprises 11 mutations selected from N297A, N297G, L234A, L235E, G237A, P238S, H268Q, H268A, V309L, A330S and P331S. In some embodiments, the bifunctional fusion protein comprises 11 mutations selected from N297A, N297Q, L234A, L235E, G237A, P238S, H268Q, H268A, V309L, A330S and P331S. In some embodiments, the bifunctional fusion protein comprises 11 mutations selected from N297A, N297Q, N297G, L235E, G237A, P238S, H268Q, H268A, V309L, A330S and P331S. In some embodiments, the bifunctional fusion protein comprises 11 mutations selected from N297A, N297Q, N297G, L234A, G237A, P238S, H268Q, H268A, V309L, A330S and P33iS. In some embodiments, the bifunctional fusion protein comprises 11 mutations selected from N297A, N297Q, N297G, L234A, L235E, P238S, H268Q, H268A, V309L, A330S and P331S. In some embodiments, the bifunctional fusion protein comprises 11 mutations selected from N297A, N297Q, N297G, L234A, L235E, G237A, H268Q, H268A, V309L, A330S and P331S. In some embodiments, the bifunctional fusion protein comprises 11 mutations selected from N297A, N297Q, N297G, L234A, L235E, G237A, P238S, H268A, V309L, A330S and P331S. In some embodiments, the bifunctional fusion protein comprises 11 mutations selected from N297A, N297Q, N297G, L234A, L235E, G237A, P238S, H268Q, V309L, A330S and P331S. In some embodiments, the bifunctional fusion protein comprises 11 mutations selected from N297A, N297Q, N297G, L234A, L235E, G237A, P238S, H268Q, H268A, A330S and P331S. In some embodiments, the bifunctional fusion protein comprises 11 mutations selected from N297A, N297Q, N297G, L234A, L235E, G237A, P238S, H268Q, H268A, V309L and A330S.

In some embodiments, the bifunctional fusion protein comprises 12 mutations comprising N297A, N297Q, N297G, L234A, L235E, G237A, P238S, H268Q, H268A, V309L, A330S and P331S.

In some embodiments of the bifunctional fusion protein, the IL-2 and IL-33 polypeptides are 10 to 100 angstrom units (Å) apart. In some embodiments of the bifunctional fusion protein, the IL-2 and IL-33 polypeptides are 10 angstrom units (Å) apart. In some embodiments of the bifunctional fusion protein, the IL-2 and IL-33 polypeptides are 20 angstrom units (Å) apart. In some embodiments of the bifunctional fusion protein, the IL-2 and IL-33 polypeptides are 30 angstrom units (Å) apart. In some embodiments of the bifunctional fusion protein, the IL-2 and IL-33 polypeptides are 40 angstrom units (Å) apart. In some embodiments of the bifunctional fusion protein, the IL-2 and IL-33 polypeptides are 50 angstrom units (Å) apart. In some embodiments of the bifunctional fusion protein, the IL-2 and IL-33 polypeptides are 60 angstrom units (Å) apart. In some embodiments of the bifunctional fusion protein, the IL-2 and IL-33 polypeptides are 70 angstrom units (Å) apart. In some embodiments of the bifunctional fusion protein, the IL-2 and IL-33 polypeptides are 80 angstrom units (Å) apart. In some embodiments of the bifunctional fusion protein, the IL-2 and IL-33 polypeptides are 90 angstrom units (Å) apart. In some embodiments of the bifunctional fusion protein, the IL-2 and IL-33 polypeptides are 100 angstrom units (Å) apart.

### Linker

In some embodiments, the bifunctional protein further comprises a linker. Each domain of the fusion protein may be linked by a linker, for example, a non-immunogenic linker that is long enough to allow free rotation of individual cytokines.

In some embodiments of the bifunctional fusion protein, the linker is a peptide linker or a chemical linker. In some embodiments, the linker is a peptide linker. In some embodiments, the linker is a chemical linker.

In some embodiments, provided herein is the bifunctional protein or the fusion protein, wherein the peptide linker is selected from GGGGS (SEQ ID NO: 29; n repeats of SEQ ID NO: 29, n=1-5), GGGGSGGGGSGGGGS (SEQ ID NO: 31), EAAAK (SEQ ID NO: 32; n repeats of SEQ ID NO: 32, n=1-3), GGGGGGGG (SEQ ID NO: 33), GSGSGSGSGS (SEQ ID NO: 34), GSGSGSGSGSGSGSGSGSGS (SEQ ID NO: 35), GGSGGSGGS (SEQ ID NO: 36), GSGGS (SEQ ID NO: 37), GSSGS (SEQ ID NO: 38), a SEG-linker, a GSAT (SEQ ID NO: 39), SSSSGSSSSG (SEQ ID NO: 40), a flexible 22 amino acid linker LEGSGQGPGSGQGSGSPGSGQG (SEQ ID NO: 41), and GGGGSEAAAK (SEQ ID NO: 42, n=1-2), EGKSSGSGSESKST (SEQ ID NO: 44), GGGGSLVPRGSGGGGS (SEQ ID NO: 45), KESGSVSSEQLAQFRSLD (SEQ ID NO: 46), GGGSEGGGSEGGGSEGGG (SEQ ID NO: 47) or rigid peptide linkers PAPAP (SEQ ID NO: 48), (Ala-Pro)ₙ, AEAAAKEAAAKA (SEQ ID NO: 49), AEAAAKEAAAKEAAAKEAAAKALEAEAAAKEAAAKEAAAKEAAAKA (SEQ ID NO: 50).

In some embodiments, the linker may be a cleavable peptide linker, such as a 2A self-cleaving peptide. Exemplary 2A peptides include T2A, P2A, F2A and E2A.

Exemplary linker sequences are provided in Table 4.

In some embodiments, the bifunctional fusion protein further comprises a tag at the N-terminus or C-terminus.

The tag herein is any affinity tag known in the art suitable for recombinant protein purification. In some embodiments, the tag is a Flag tag. In some embodiments, the tag is a His tag. In some embodiments, the tag is an HA tag. In some embodiments, the tag is a CBP tag. In some embodiments, the tag is a MBP tag. In some embodiments, the tag is a GST tag. In some embodiments, the tag is a Myc tag. In some embodiments, the tag is a SUMO tag. In some embodiments, the tag is a TAP tag. In some embodiments, the tag is a V5 tag. In some embodiments, the tag is a TRX tag.

In some embodiments, the bifunctional fusion protein comprises the sequence of any one of SEQ ID NO: 54 to SEQ ID NO:75 and SEQ ID NO: 80 to SEQ ID NO: 81.

In some embodiments, the fusion protein is lyophilized.

In some embodiments, provided herein is a polynucleotide encoding the IL-33 polypeptide or the bifunctional fusion protein described herein.

### Gene Therapy

In some embodiments, provided herein is a polynucleotide encoding the bifunctional protein or the bifunctional fusion protein described herein. In some embodiments, provided herein is a gene therapy vector comprising the polynucleotide described herein.

In some embodiments, the gene therapy vector is a viral vector or a non-viral vector. In some embodiments, the gene therapy vector is a viral vector. In some embodiments, the gene therapy vector is a non-viral vector.

In some embodiments, the gene therapy vector is a viral vector, including, for example and without limitation, adeno-associated virus vectors (AAV), lentiviral vectors, and adenovirus vectors. In some embodiments, the viral vector is without limitation, helper-dependent adenoviral, hybrid adenoviral, herpes simplex virus, poxvirus, Epstein-Barr virus, vaccinia virus, and human cytomegalovirus vector, including recombinant versions thereof.

Gene therapy vectors provide a means for delivering nucleic acids into a broad range of cells, including dividing and non-dividing cells. Gene therapy vectors can be employed to deliver a nucleic acid of interest to a cell in vitro, e.g., for ex vivo gene therapy. The vectors are additionally useful in a method of delivering a nucleic acid to a subject in need thereof, e.g., to express an immunogenic or therapeutic polypeptide or a functional RNA, thus the polypeptide or functional RNA can be produced in vivo in the subject.

Gene therapy vectors are used to treat and/or prevent any disease state for which it is beneficial to deliver a therapeutic polypeptide or functional nucleic acid.

The nucleic acid delivery vectors may also be employed to provide a functional nucleic acid to a cell in vitro or in vivo. Expression of the functional nucleic acid in the cell, for example, can diminish expression of a particular target protein by the cell, e.g., a downstream target in a signaling pathway. Accordingly, functional nucleic acid can be administered to decrease expression of a particular protein in a subject in need thereof. The nucleic acid delivery vectors can also be used for the purpose of evaluating safety (spread, toxicity, immunogenicity, etc.).

### Delivery

The nucleic acids described herein encoding IL-2 and IL-33 cytokines, can be delivered to a cell of interest by various delivery systems such as vectors, e.g., plasmids and delivery vectors.

In some embodiments, the cytokine variants and/or bifunctional fusion proteins can be delivered by nanoparticles, which can be organic or inorganic. Nanoparticles are well known in the art. Any suitable nanoparticle design can be used to deliver cytokines or nucleic acids encoding cytokines. For instance, organic (e.g. lipid and/or polymer) nanoparticles can be suitable for use as delivery vehicles in certain embodiments of this disclosure. Exemplary lipids for use in nanoparticle formulations, and/or gene transfer are shown in Table 1 (below).

In some embodiments, provided herein is a microparticle comprising the polynucleotide or the bifunctional fusion protein described herein. In some embodiments, the microparticle is a liposome, nanoparticle, microsphere, nanosphere, microcapsule, nanocapsule, extracellular vesicle or a lipid nanoparticle. In some embodiments, microparticles comprise poly(lactide-co-glycolide), aliphatic polyesters including, but not limited to, poly-glycolic acid and poly-lactic acid, hyaluronic acid, modified polysaccharides, chitosan, cellulose, dextran, polyurethanes, polyacrylic acids, pseudo-poly(amino acids), polyhydroxybutrate-related copolymers, polyanhydrides, polymethylmethacrylate, poly(ethylene oxide), lecithin and phospholipids. In some embodiments, microparticles are used for a controlled release of therapeutic agent.

In some embodiments, delivery is by any one of the means listed in Table 1 below, as non-limiting examples.

TABLE 1
Modes of delivery of Recombinant IL-33, IL-2 or Fusions Thereof

| | | Delivery into | | | Type of |
|---|---|---|---|---|---|
| | | Non-Dividing | Duration of | Genome | Molecule |
| Delivery | Vector/Mode | Cells | Expression | Integration | Delivered |
| Physical | (e.g., | YES | Transient | NO | Nucleic Acids |
| | electroporation, | | | | and Proteins |
| | particle gun, | | | | |
| | Calcium | | | | |
| | Phosphate | | | | |
| | transfection | | | | |
| Viral | Retrovirus | NO | Stable | YES | RNA |
| | Lentivirus | YES | Stable | YES/NO with | RNA |
| | | | | modification | |
| | Adenovirus | YES | Transient | NO | DNA |
| | Adeno- | YES | Stable | NO | DNA |
| | Associated | | | | |
| | Virus (AAV) | | | | |
| | Vaccinia Virus | YES | Very | NO | DNA |
| | | | Transient | | |
| | Herpes Simplex | YES | Stable | NO | DNA |
| | Virus | | | | |
| Non-Viral | Cationic | YES | Transient | Depends on | Nucleic Acids |
| | Liposomes | | | what is | and Proteins |
| | | | | delivered | |
| | Polymeric | YES | Transient | Depends on | Nucleic Acids |
| | Nanoparticles | | | what is | and Proteins |
| | | | | delivered | |
| Biological | Attenuated | YES | Transient | NO | Nucleic Acids |
| Non-Viral | Bacteria | | | | |
| | Engineered | | | | |
| | Lactobacilli | YES | Transient | NO | Nucleic Acids |
| Delivery | Engineered | YES | Transient | NO | Nucleic Acids |
| Vehicles | Bacteriophages | | | | |
| | Mammalian | YES | Transient | NO | Nucleic Acids |
| | Virus-like | | | | |
| | Particles | | | | |
| | Biological | YES | Transient | NO | Nucleic Acids |
| | liposomes: | | | | |
| | Erythrocyte | | | | |
| | Ghosts and | | | | |
| | Exosomes | | | | |

Any suitable promoter can be used to drive expression of cytokines IL-33 and IL-2. A promoter used to drive expression of cytokines can include AAV ITR, which eliminates the need for an additional promoter element in a compact vector. ITR activity is relatively weak, so it can be used to reduce potential toxicity due to over expression of the chosen nuclease. For ubiquitous expression, promoters that can be used include CMV, CAG, CBh, PGK, SV40, Ferritin heavy or light chains, etc. For brain or other CNS cell expression, suitable promoters can include: SynapsinI for all neurons, CaMKIIalpha for excitatory neurons, GAD67 or GAD65 or VGAT for GABAergic neurons, etc. For liver cell expression, suitable promoters include the Albumin promoter. For lung cell expression, suitable promoters can include SP-B. For endothelial cells, suitable promoters can include ICAM. For hematopoietic cells suitable promoters can include IFNbeta or CD45. For osteoblasts, suitable promoters can include OG-2.

In some cases, separate promoters drive expression of the IL-2 and IL-33 within the same nucleic acid molecule. For instance, a vector or viral vector can comprise a first promoter operably linked to a nucleic acid encoding IL-2 and a second promoter operably linked to a nucleic acid encoding IL-33. Promoters include Pol III promoters such as U6 or H1.

IL-33 variants or bifunctional fusion proteins comprising IL-33 and IL-2 can be delivered using adeno associated virus (AAV), lentivirus, adenovirus or other plasmid or viral vector types, in particular, using formulations and doses from, for example, U.S. Pat. No. 8,454,972 (formulations, doses for adenovirus), U.S. Pat. No. 8,404,658 (formulations, doses for AAV) and U.S. Pat. No. 5,846,946 (formulations, doses for DNA plasmids) and from clinical trials and publications regarding the clinical trials involving lentivirus, AAV and adenovirus. For example, for AAV, the route of administration, formulation and dose can be as in U.S. Pat. No. 8,454,972 and as in clinical trials involving AAV. For Adenovirus, the route of administration, formulation and dose can be as in U.S. Pat. No. 8,404,658 and as in clinical trials involving adenovirus. For plasmid delivery, the route of administration, formulation and dose can be as in U.S. Pat. No. 5,846,946 and as in clinical studies involving plasmids. Doses can be based on or extrapolated to an average 70 kg individual (e.g., a male adult human), and can be adjusted for patients, subjects, mammals of different weight and species. Frequency of administration is within the ambit of the medical or veterinary practitioner (e.g., physician, veterinarian), depending on usual factors including the age, sex, general health, other conditions of the patient or subject and the particular condition or symptoms being addressed. The viral vectors can be injected into the tissue of interest. For cell-type specific base editing, the expression of the base editor and optional guide nucleic acid can be driven by a cell-type specific promoter.

For in vivo delivery, AAV can be advantageous over other viral vectors. In some cases, AAV allows low toxicity, which can be due to the purification method not requiring ultra-centrifugation of cell particles that can activate the immune response. In some cases, AAV allows low probability of causing insertional mutagenesis because it doesn't integrate into the host genome. An AAV can be AAV1, AAV2, AAV4, AAV5, AAV8, AAV9 or any combination thereof. One can select the type of AAV with regard to the cells to be targeted; e.g., one can select AAV serotypes 1, 2, 5, 8, 9 or a hybrid capsid AAV1, AAV2, AAV5 or any combination thereof for targeting brain or neuronal cells; and one can select AAV4 for targeting cardiac tissue. AAV8 is useful for delivery to the liver. A tabulation of certain AAV serotypes as to these cells can be found in Grimm, D. et al, J. Virol. 82: 5887-5911 (2008)).

Lentiviruses are complex retroviruses that have the ability to infect and express their genes in both mitotic and post-mitotic cells. The most commonly known lentivirus is the human immunodeficiency virus (HIV), which uses the envelope glycoproteins of other viruses to target a broad range of cell types. In another embodiment, minimal non-primate lentiviral vectors based on the equine infectious anemia virus (EIAV) are also contemplated. In another embodiment, RetinoStat®, an equine infectious anemia virus-based lentiviral gene therapy vector that expresses angiostatic proteins endostatin and angiostatin that is contemplated to be delivered via a subretinal injection. In another embodiment, use of self-inactivating lentiviral vectors is contemplated.

Any RNA of the systems, for example an IL-33 or IL-2 encoding mRNA, or an mRNA encoding the bifunctional fusion protein can be delivered in the form of RNA. mRNA can be generated using in vitro transcription, for example, from a PCR cassette containing the following elements: T7 promoter, optional kozak sequence, nuclease sequence, and 3′ UTR such as a 3′ UTR from beta globin-polyA tail. The cassette can be used for transcription by T7 polymerase.

To enhance expression and reduce possible toxicity, the nucleotide sequence can be modified to include one or more modified nucleoside e.g. using pseudo-U or 5-Methyl-C.

The system can comprise one or more different vectors. In an aspect, the IL-33 and IL-2 nucleic acids are codon optimized for expression the desired cell type, preferentially a eukaryotic cell, preferably a mammalian cell or a human cell.

In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/(visited Jul. 9, 2002), and these tables can be adapted in a number of ways. See, Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g. 1, 2, 3, 4, 5, 10, 15, 20, 25, 50, or more, or all codons) in a sequence encoding an engineered nuclease correspond to the most frequently used codon for a particular amino acid.

Packaging cells are typically used to form virus particles that are capable of infecting a host cell. Such cells include 293 cells, which package adenovirus, and psi.2 cells or PA317 cells, which package retrovirus. Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA can be packaged in a cell line, which contains a helper plasmid encoding the other AAV genes, namely rep and cap, but lacking ITR sequences. The cell line can also be infected with adenovirus as a helper. The helper virus can promote replication of the AAV vector and expression of AAV genes from the helper plasmid. The helper plasmid in some cases is not packaged in significant amounts due to a lack of ITR sequences. Contamination with adenovirus can be reduced by, e.g., heat treatment to which adenovirus is more sensitive than AAV.

### Regulatory T Cells (Tregs) in Suppressing Autoimmune Inflammation

Regulatory T cells (Treg) cells are FOXP3+CD4+CD25+ cells that play an important role in maintaining self-tolerance and normal immune homeostasis and suppressing autoimmune inflammation. Current immunosuppressive therapeutics generally target individual proinflammatory pathways and often exhibit partial efficacy or are applicable only to specific diseases. The present invention provides a method to suppress autoimmune disease involving increased selective production and activation of natural suppressor cells.

Described herein are therapeutic agents that selectively promote T-reg cell proliferation, survival, activation and/or function. By “selectively promote,” it is meant the therapeutic agent promotes the activity in T-reg cells but has limited or lacks the ability to promote activity in non-regulatory T cells. The methods and compositions provided herein promote these activities of T-reg cell growth/survival but have a reduced ability, as compared to wild-type IL-33 and/or IL-2, to promote non-regulatory T-cell (FOXP3 CD25−) and NK cell proliferation, survival, activation and/or function, thus minimizing side-effects, providing an improved safer autoimmune therapy.

### ST2+ Treg Cells

Tregs are robustly activated by IL-2, but IL-2 also activates many other cell types, which can result in significant toxicity. Subsets of Tregs are defined by expression of specific molecular markers or by their roles in different immunological responses. One Treg subset is the ST2+ Treg subset, also interchangeably referred to herein as reparative Treg cells, suppressive Treg cells or tissue resident Treg cells. ST2+ Tregs contain the ST2 cell surface marker, which is a component of a cytokine receptor also known interleukin 1 receptor-like 1 protein (IL1RL1), which is a subunit of the IL-33 receptor. IL-33 is an immunomodulatory cytokine associated with acute inflammatory responses, also referred to as alarmin. ST2+ Tregs are found in tissues such as muscle, visceral adipose, colon, and lung, and possess immunoregulatory and tissue repair functions.

In some embodiments, provided herein is a method for modulating regulatory T cells (Treg) in a subject comprising administering to the subject the bifunctional protein or the fusion protein, or the composition disclosed herein.

In some embodiments, the method expands ST2+ Treg cells, CD25^{bright} Treg cells and/or circulating Foxp3+ Treg and Foxp3-negative Tr1 or Th3 cells. In some embodiments, the method expands ST2+ Treg cells. In some embodiments, the method expands CD25^{bright} Treg cells. In some embodiments, the method expands circulating Foxp3+ Treg cells. In some embodiments, the method expands Foxp3-negative Tr1 and Th3 cells. In some embodiments, the method expands ST2-negative regulatory T-cells, that may have down-regulated the expression of ST2 after activation or have been activated by the ST2+ Tregs in a bystander manner.

In some embodiments, the ST2+ Treg cells comprise ST2⁺CD25^{bright} double positive Tregs.

In some embodiments, a proportion of ST2+ Treg and/or ST2⁺CD25^{bright} double positive Tregs traffic to a tissue at the site of disease.

In some embodiments, the method increases immune tolerance in the tissue microenvironment.

In some embodiments, provided herein is a method for promoting local immune homeostasis in a tissue microenvironment to support Treg durability in a subject comprising administering to the subject the bifunctional protein or the fusion protein, or the composition disclosed herein.

In some embodiments of the method, the fusion protein upregulates ST2+ Treg cells in the tissue microenvironment.

In some embodiments, the ST2+ Treg cells comprise ST2⁺CD25^{bright} double positive Treg cells.

In some embodiments, the fusion protein further activates M2 macrophages, eosinophils, type 2 innate lymphoid cells (ILC2), and/or Th17+ Treg cells in the tissue microenvironment.

In some embodiments, the fusion protein inhibits NK cells, M1 macrophages, T effector cells, and/or pro-inflammatory cytokines.

In some embodiments, the fusion protein further decreases immunogenic response in the tissue.

In some embodiments, the tolerogenic tissue environment restores durable response to defend a disease.

In some embodiments, the durable immune response modulates inflammatory suppression in the tissues at the site of disease.

In some embodiments, provided herein is a method for selectively expanding ST2+ Treg cells in a subject comprising administering to the subject a bifunctional protein comprising an IL-2 variant associated with an IL-33 variant, wherein the IL-2 variant has amino acid substitutions N88D and the IL-33 variant has amino acid substitutions C116F, wherein the administration of the bifunctional protein results in eosinophil level at less than 5 fold, less than 4 fold, less than 3 fold or less than 2 fold as compared to the level before the treatment.

In some embodiments of the method, the IL-2 variant has amino acid substitutions N88D and C125S and the IL-33 variant has amino acid substitutions C97G, C116F, C121S and/or C148G. In some embodiments, the IL-2 variant has amino acid substitutions N88D and C125S and the IL-33 variant has amino acid substitutions selected from: (i) C97G and C116F; (ii) C97G, C116F and C148G; and (iii) C97G, C116F, C121S and C148G.

In some embodiments, the administration of the bifunctional protein results in eosinophil level at less than 3 fold or less than 2 fold as compared to the level before the treatment.

In some embodiments, provided herein is a method of treating an autoimmune or inflammatory disease in a subject comprising administering to the subject the bifunctional protein or the fusion protein, or the composition disclosed herein.

In some embodiments, the disease is an autoimmune disease.

In some embodiments, provided herein is a population of Treg cells generated by contacting a T cell containing sample with the bifunctional protein or the fusion protein, or the composition disclosed herein.

In some embodiments, provided herein is a population of Treg cells, wherein the Treg cells are ST2+ Treg cells.

In some embodiments, the T cell containing sample is a blood sample, a cell culture, or an iPSC-derived cell sample.

In some embodiments, the ST2+ cells are ST2+T regulatory cells, tissue resident ST2+ cells or a combination of tissue resident ST2+ and CD25^{bright} cells. In some embodiments, the ST2+ cells are ST2+T regulatory cells. In some embodiments, the ST2+ cells are tissue resident ST2+ cells. In some embodiments, the ST2+ cells comprise a combination of tissue resident ST2+ and CD25^{bright} cells.

### Engineered Host Cell

The disclosure in some embodiments provides a method of modifying a cell or organism. The cell can be a prokaryotic cell or a eukaryotic cell. The cell can be a mammalian cell. The mammalian cell many be a non-human primate, bovine, porcine, rodent or mouse cell. The modification introduced to the cell by the IL-2 and IL-33 of the present disclosure can be such that the cell and progeny of the cell are altered for improved cytokine expression or other desired cellular output. The modification introduced to the cell by the methods of the present disclosure can be such that the cell and progeny of the cell include an alteration that changes the biologic product produced. In some embodiments, the engineered host cell is used for manufacturing recombinant protein. In some embodiments, the engineered host cell is used for ex vivo therapy.

In some embodiments, provided herein is an engineered host cell comprising the polynucleotide or the vector described herein.

In some embodiments, the cell is a mammalian cell. In some embodiments, the cell is a Chinese hamster ovary (CHO) cell.

In some embodiments, the cell is a bacterial cell. In some embodiments, the cell is an E. coli cell.

In some embodiments, the cell is an insect cell. In some embodiments, the cell is a yeast cell.

### Pharmaceutical Composition

In some embodiments, provided herein is a pharmaceutical composition comprising the bifunctional fusion protein described herein.

Aspects of the present disclosure relate to pharmaceutical compositions comprising IL-2 and/or IL-33 variants or nucleic acids or vectors encoding the same. The term “pharmaceutical composition”, as used herein, refers to a composition formulated for pharmaceutical use. In some embodiments, the pharmaceutical composition further comprises a pharmaceutically acceptable carrier. In some embodiments, the pharmaceutical composition comprises additional agents (e.g., for specific delivery, increasing half-life, or other therapeutic compounds). As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the compound from one site (e.g., the delivery site) of the body, to another site (e.g., organ, tissue or portion of the body). A pharmaceutically acceptable carrier is “acceptable” in the sense of being compatible with the other ingredients of the formulation and not injurious to the tissue of the subject (e.g., physiologically compatible, sterile, physiologic pH, etc.).

Some nonlimiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient,” “carrier,” “pharmaceutically acceptable carrier,” “vehicle,” or the like are used interchangeably herein.

Pharmaceutical compositions can comprise one or more pH buffering compounds to maintain the pH of the formulation at a predetermined level that reflects physiological pH, such as in the range of about 5.0 to about 8.0. The pH buffering compound used in the aqueous liquid formulation can be an amino acid or mixture of amino acids, such as histidine or a mixture of amino acids such as histidine and glycine. Alternatively, the pH buffering compound is preferably an agent which maintains the pH of the formulation at a predetermined level, such as in the range of about 5.0 to about 8.0, and which does not chelate calcium ions. Illustrative examples of such pH buffering compounds include, but are not limited to, imidazole and acetate ions. The pH buffering compound may be present in any amount suitable to maintain the pH of the formulation at a predetermined level.

Pharmaceutical compositions can also contain one or more osmotic modulating agents, i.e., a compound that modulates the osmotic properties (e.g., tonicity, osmolality, and/or osmotic pressure) of the formulation to a level that is acceptable to the blood stream and blood cells of recipient individuals. The osmotic modulating agent can be an agent that does not chelate calcium ions. The osmotic modulating agent can be any compound known or available to those skilled in the art that modulates the osmotic properties of the formulation. One skilled in the art may empirically determine the suitability of a given osmotic modulating agent for use in the inventive formulation. Illustrative examples of suitable types of osmotic modulating agents include, but are not limited to: salts, such as sodium chloride and sodium acetate; sugars, such as sucrose, dextrose, and mannitol; amino acids, such as glycine; and mixtures of one or more of these agents and/or types of agents. The osmotic modulating agent(s) may be present in any concentration sufficient to modulate the osmotic properties of the formulation.

In some embodiments, the pharmaceutical composition is formulated for delivery to a subject, e.g., for gene editing. Suitable routes of administrating the pharmaceutical composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration.

In some embodiments, the pharmaceutical composition described herein is administered locally to a diseased site. In some embodiments, the pharmaceutical composition described herein is administered to a subject by injection, by means of a catheter, by means of a suppository, or by means of an implant, the implant being of a porous, non-porous, or gelatinous material, including a membrane, such as a sialastic membrane, or a fiber.

In other embodiments, the pharmaceutical composition described herein is delivered in a controlled release system. In one embodiment, a pump can be used (See, e.g., Langer, 1990, Science 249: 1527-1533; Sefton, 1989, CRC Crit. Ref Biomed. Eng. 14:201; Buchwald et al., 1980, Surgery 88:507; Saudek et al., 1989, N. Engl. J. Med. 321:574). In another embodiment, polymeric materials can be used. (See, e.g., Medical Applications of Controlled Release (Langer and Wise eds., CRC Press, Boca Raton, Fla., 1974); Controlled Drug Bioavailability, Drug Product Design and Performance (Smolen and Ball eds., Wiley, New York, 1984); Ranger and Peppas, 1983, Macromol. Sci. Rev. Macromol. Chem. 23:61. See also Levy et al., 1985, Science 228: 190; During et al., 1989, Ann. Neurol. 25:351; Howard et ah, 1989, J. Neurosurg. 71: 105.) Other controlled release systems are discussed, for example, in Langer, supra.

In some embodiments, the pharmaceutical composition is formulated in accordance with routine procedures as a composition adapted for intravenous or subcutaneous administration to a subject, e.g., a human. In some embodiments, pharmaceutical composition for administration by injection are solutions in sterile isotonic use as solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical is to be administered by infusion, it can be dispensed with an infusion bottle containing sterile pharmaceutical grade water or saline. Where the pharmaceutical composition is administered by injection, an ampoule of sterile water for injection or saline can be provided so that the ingredients can be mixed prior to administration.

A pharmaceutical composition for systemic administration can be a liquid, e.g., sterile saline, lactated Ringer's or Hank's solution. In addition, the pharmaceutical composition can be in solid forms and re-dissolved or suspended immediately prior to use. Lyophilized forms are also contemplated. The pharmaceutical composition can be contained within a lipid particle or vesicle, such as a liposome or microcrystal, which is also suitable for parenteral administration. The particles can be of any suitable structure, such as unilamellar or plurilamellar, so long as compositions are contained therein. Compounds can be entrapped in “stabilized plasmid-lipid particles” (SPLP) containing the fusogenic lipid dioleoylphosphatidylethanolamine (DOPE), low levels (5-10 mol %) of cationic lipid, and stabilized by a polyethyleneglycol (PEG) coating (Zhang Y. P. et ah, Gene Ther. 1999, 6: 1438-47). Positively charged lipids such as N-[1-(2,3-dioleoyloxi)propyl]-N,N,N-trimethyl-amoniummethylsulfate, or “DOTAP,” are particularly preferred for such particles and vesicles. The preparation of such lipid particles is well known. See, e.g., U.S. Pat. Nos. 4,880,635; 4,906,477; 4,911,928; 4,917,951; 4,920,016; and 4,921,757; each of which is incorporated herein by reference.

The pharmaceutical composition described herein can be administered or packaged as a unit dose, for example. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier, or vehicle.

Further, the pharmaceutical composition can be provided as a pharmaceutical kit comprising (a) a container containing a compound of the invention in lyophilized form and (b) a second container containing a pharmaceutically acceptable diluent (e.g., sterile used for reconstitution or dilution of the lyophilized compound of the invention. Alternatively, the pharmaceutical composition is in a prefilled syringe. Optionally associated with such container(s) can be a notice in the form prescribed by a governmental agency regulating the manufacture, use or sale of pharmaceuticals or biological products, which notice reflects approval by the agency of manufacture, use or sale for human administration.

In another aspect, an article of manufacture containing materials useful for the treatment of the diseases described above is included. In some embodiments, the article of manufacture comprises a container and a label. Suitable containers include, for example, bottles, vials, syringes, and test tubes. The containers can be formed from a variety of materials such as glass or plastic. In some embodiments, the container holds a composition that is effective for treating a disease described herein and can have a sterile access port. For example, the container can be an intravenous solution bag or a vial having a stopper pierceable by a hypodermic injection needle. The active agent in the composition is a compound of the invention. In some embodiments, the label on or associated with the container indicates that the composition is used for treating the disease of choice. The article of manufacture can further comprise a second container comprising a pharmaceutically-acceptable buffer, such as phosphate-buffered saline, Ringer's solution, or dextrose solution. It can further include other materials desirable from a commercial and user standpoint, including other buffers, diluents, filters, needles, syringes, and package inserts with instructions for use.

In some embodiments, the IL-33 and IL-2 and/or bifunctional proteins or nucleic acids or RNA encoding the same are provided as part of a pharmaceutical composition. In some embodiments, the pharmaceutical composition comprises any of the fusion proteins provided herein (e.g., fusion of IL-2 and IL-3 polypeptides, including variants thereof, the fusion comprising, for example, a linker and half-life extension moiety). In some embodiments, the pharmaceutical composition comprises any of the complexes provided herein. In some embodiments, the pharmaceutical composition comprises a nucleic acid encoding IL-2 and IL-33. In some embodiments, the pharmaceutical composition comprises a messenger RNA encoding IL-2 and IL-33. In some embodiments, the pharmaceutical composition comprises a circular messenger RNA encoding IL-2 and IL-33. Pharmaceutical compositions can optionally comprise one or more additional therapeutically active substances.

In some embodiments, provided herein is a method of treating autoimmune disease, the method comprising administering to a subject in need thereof a therapeutically effective dose of the bifunctional fusion protein or the pharmaceutical composition or the engineered cell described herein.

General considerations in the formulation and/or manufacture of pharmaceutical agents may be found, for example, in Remington: The Science and Practice of Pharmacy 21^{st} ed., Lippincott Williams & Wilkins, 2005 (incorporated herein by reference).

Treating Autoimmune and/or Inflammatory Disease or Disorders

In some embodiments, administration of the composition removes or reduces the effect of antibodies in a subject. In some embodiments, the subject has an autoimmune disease. In some embodiments, the antibodies are pathogenic autoantibodies. In some embodiments, administration of the composition is used to treat an autoimmune disorder.

Autoimmune and/or inflammatory diseases, disorders, or conditions may be amenable to treatment with or may be prevented by administration of IL-33 and IL-2 cytokines that synergistically promote ST2+ Treg proliferation and/or activity in a subject. In some embodiments, one or more IL-33 and IL-2 variants described herein are used to treat an autoimmune disease, disorder or condition.

Such diseases, disorders, and conditions that may be diminished in onset and/or severity include, but are not limited to, inflammation, autoimmune disease, paraneoplastic autoimmune diseases, cartilage inflammation, fibrotic disease and/or bone degradation, arthritis, rheumatoid arthritis, juvenile arthritis, juvenile rheumatoid arthritis, pauciarticular juvenile rheumatoid arthritis, polyarticular juvenile rheumatoid arthritis, systemic onset juvenile rheumatoid arthritis, juvenile ankylosing spondylitis, juvenile enteropathic arthritis, juvenile reactive arthritis, juvenile Reter's Syndrome, SEA Syndrome (Seronegativity, Enthesopathy, Arthropathy Syndrome), juvenile dermatomyositis, juvenile psoriatic arthritis, juvenile scleroderma, juvenile systemic lupus erythematosus, juvenile vasculitis, pauciarticular rheumatoid arthritis, polyarticular rheumatoid arthritis, systemic onset rheumatoid arthritis, ankylosing spondylitis, enteropathic arthritis, reactive arthritis, Reter's Syndrome, SEA Syndrome (Seronegativity, Enthesopathy, Arthropathy Syndrome), dermatomyositis, psoriatic arthritis, scleroderma, systemic lupus erythematosus, vasculitis, myolitis, polymyolitis, dermatomyolitis, osteoarthritis, polyarteritis nodossa, Wegener's granulomatosis, arteritis, poloymyalgia rheumatica, sarcoidosis, scleroderma, sclerosis, primary biliary sclerosis, sclerosing cholangitis, Sjogren's syndrome, atopic dermatitis, IgA nephropathy, membranous nephropathy, psoriasis, plaque psoriasis, guttate psoriasis, inverse psoriasis, pustular psoriasis, erythrodermic psoriasis, dermatitis, atopic dermatitis, atherosclerosis, lupus, Still's disease, Systemic Lupus Erythematosus (SLE), myasthenia gravis, inflammatory bowel disease (IBD), Crohn's disease, ulcerative colitis, celiac disease, multiple sclerosis (MS), asthma, COPD, Guillain-Barre disease, Type I diabetes mellitus, thyroiditis (e.g., Graves' disease), Addison's disease, Raynaud's phenomenon, autoimmune hepatitis, GVHD, transplantation rejection, and the like. In specific embodiments, pharmaceutical compositions comprising a therapeutically effective amount of an IL-33 and IL-2 fusion protein are provided.

In some embodiments, the disease is selected from a group consisting of MS, lupus, ankylosing spondylitis, arthritis, colitis, Type I diabetes, mitigate severity of inflammatory disease, Crohn's disease, heart disease, mitigate complications from immune response in pregnancy, mitigate complications from graft-versus-host disease (GVHD), mitigate severity of allergies, reduce rejection of HSC or allogeneic solid organ transplant, depression, ALS and/or myasthenia gravis.

The term “treatment” encompasses alleviation or prevention of at least one symptom or other aspect of a disorder, or reduction of disease severity, and the like. An ST2+T-reg-selective IL-33 variant or fusion protein comprising IL-33 variant and IL-2 together with a half-life extension moiety need not effect a complete cure, or eradicate every symptom or manifestation of a disease, to constitute a viable therapeutic agent. As is recognized in the pertinent field, drugs employed as therapeutic agents may reduce the severity of a given disease state but need not abolish every manifestation of the disease to be regarded as useful therapeutic agents. Similarly, a prophylactically administered treatment need not be completely effective in preventing the onset of a condition in order to constitute a viable prophylactic agent. Simply reducing the impact of a disease (for example, by reducing the number or severity of its symptoms, or by increasing the effectiveness of another treatment, or by producing another beneficial effect), or reducing the likelihood that the disease will occur or worsen in a subject, is sufficient. One embodiment of the invention is directed to a method comprising administering to a patient an ST2+T-reg-selective bifunctional protein comprising IL-33 variant, IL-2 polypeptide and a half-life extension moiety in an amount and for a time sufficient to prevent or treat i.e. induce a sustained improvement over baseline of an indicator that reflects the severity of the particular disorder.

In some embodiments, provided herein is a method of treating autoimmune disease, the method comprising administering to a subject in need thereof a therapeutically effective dose of the bifunctional fusion protein, the pharmaceutical composition or the engineered cell described herein. In some embodiments, the administration is subcutaneous, intramuscular or intravenous. In some embodiments, the administration is subcutaneous, intramuscular or intravenous. In some embodiments, the administration is subcutaneous. In some embodiments, the administration is intramuscular. In some embodiments, the administration is intravenous. Other routes of administration are discussed in greater detail in another section.

In some embodiments, the administration is subcutaneous. In some embodiments, the administration stimulates proliferation and/or activation of immune cells.

In some embodiments, the administration stimulates proliferation and/or activation of immune cells. In some embodiments, the immune cells are ST2+ cells, Foxp3− ST2+ cells, Foxp3− ST2− Tr1 cells, Th3 cells, ILC2, macrophages, MDSCs, regulatory B cells, B1B cells, or tolerogenic dendritic cells.

### Routes of Administration

A bifunctional protein described herein (or a composition or medicament containing a recombinant bifunctional protein described herein) can be administered by any appropriate route. In some embodiments, a recombinant IL-33 variant or recombinant IL-2 and IL-33 variant bifunctional fusion protein or a pharmaceutical composition containing the same is administered systemically. Systemic administration may be intravenous, intradermal, inhalation, transdermal (topical), intraocular, intramuscular, subcutaneous, intramuscular, oral and/or transmucosal administration. In some embodiments, a recombinant IL-33 variant or recombinant IL-2 and IL-33 variant bifunctional fusion protein or a pharmaceutical composition containing the same is administered subcutaneously. As used herein, the term “subcutaneous tissue”, is defined as a layer of loose, irregular connective tissue immediately beneath the skin. For example, the subcutaneous administration may be performed by injecting a composition into areas including, but not limited to, the thigh region, abdominal region, gluteal region, or scapular region. In some embodiments, a recombinant IL-33 variant or recombinant IL-2 and IL-33 variant bifunctional fusion protein or a pharmaceutical composition containing the same is administered intravenously. In some embodiments, a recombinant IL-33 variant or recombinant IL-2 and IL-33 variant bifunctional fusion protein or a pharmaceutical composition containing the same is administered orally. In some embodiments, a recombinant IL-33 variant or recombinant IL-2 and IL-33 variant bifunctional fusion protein or a pharmaceutical composition containing the same is administered intramuscularly. In some embodiments, more than one route can be used concurrently.

In some embodiments, administration results only in a localized effect in an individual, while in other embodiments, administration results in effects throughout multiple portions of an individual, for example, systemic effects. Typically, administration results in delivery of a recombinant IL-33 variant or bifunctional fusion protein systemically. In some embodiments, the recombinant IL-33 variant or bifunctional fusion protein is delivered to one or more target tissues including, but not limited to, heart, brain, spinal cord, striated muscle (e.g., skeletal muscle), smooth muscle, kidney, liver, lung, and/or spleen.

Combination therapies: In further embodiments, IL-2 and IL-33 variants or fusion proteins comprising the same described herein are administered in combination with other agents useful for treating a condition with which the patient is afflicted. Examples of such agents include both proteinaceous and non-proteinaceous drugs. When multiple therapeutics are co-administered, dosages may be adjusted accordingly, as is recognized in the pertinent art. “Co-administration” and combination therapy are not limited to simultaneous administration, but also include treatment regimens in which a T-reg-selective JL-2 and/or IL-33 variant is administered at least once during a course of treatment that involves administering at least one other therapeutic agent to the patient. In certain embodiments, an IL-2 or IL-33 variant is administered in combination with an inhibitor of the PI3-K/AKT/mTOR pathway, e.g., rapamycin (Rapamune, sirolimus). Inhibitors of this pathway in combination with IL-2 and/or IL-33 favor T-reg enrichment.

Although methods and materials similar or equivalent to those described herein can be used in the practice or testing of the present invention, suitable methods and materials are described below. All publications, patent applications, patents, and other references mentioned herein are incorporated by reference in their entirety. The references cited herein are not admitted to be prior art to the claimed invention. In addition, the materials, methods, and examples are illustrative only and are not intended to be limiting.

Sequences provided herein:

TABLE 2
Sequences of IL-2 variants

| | | |
|---|---|---|
| SEQ ID NO: 1 | human IL-2 | MYRMQLLSCIALSLALVTNSAPTSSSTKKTQLQLEHLLLDLQ |
| | with signal | MILNGINNYKNPKLTRMLTFKFYMPKKATELKHLQCLEEELK |
| | peptide | PLEEVLNLAQSKNFHLRPRDLISNINVIVLELKGSETTFMCE |
| | | YADETATIVEFLNRWITFCQSIISTLT |
| SEQ ID NO: 2 | wild type human | APTSSSTKKTQLQLEHLLLDLQMILNGINNYKNPKLTRMLTF |
| | IL-2 (mature) | KFYMPKKATELKHLQCLEEELKPLEEVLNLAQSKNFHLRPRD |
| | | LISNINVIVLELKGSETTFMCEYADETATIVEFLNRWITFCQ |
| | | SIISTLT |
| SEQ ID NO: 3 | human IL-2 | APTSSSTKKTQLQLEHLLLDLQMILNGINNYKNPKLTRMLTF |
| | V69A | KFYMPKKATELKHLQCLEEELKPLEEALNLAQSKNFHLRPRD |
| | | LISNINVIVLELKGSETTFMCEYADETATIVEFLNRWITFCQ |
| | | SIISTLT |
| SEQ ID NO: 4 | human IL-2 | APTSSSTKKTQLQLEHLLLDLQMILNGINNYKNPKLTRMLTF |
| | Q74P | KFYMPKKATELKHLQCLEEELKPLEEVLNLAQSKNFHLRPRD |
| | | LISNINVIVLELKGSETTFMCEYADETATIVEFLNRWITFCQ |
| | | SIISTLT |
| SEQ ID NO: 5 | human IL-2 | APTSSSTKKTQLQLEHLLLDLQMILNGINNYKNPKLTRMLTF |
| | N88D | KFYMPKKATELKHLQCLEEELKPLEEVLNLAQSKNFHLRPRD |
| | | LISDINVIVLELKGSETTFMCEYADETATIVEFLNRWITFCQ |
| | | SIISTLT |
| SEQ ID NO: 6 | human IL-2 | APTSSSTKKTQLQLEHLLLDLQMILNGINNYKNPKLTRMLTF |
| | N88R | KFYMPKKATELKHLQCLEEELKPLEEVLNLAQSKNFHLRPRD |
| | | LISRINVIVLELKGSETTFMCEYADETATIVEFLNRWITFCQ |
| | | SIISTLT |
| SEQ ID NO: 7 | human IL-2 | APTSSSTKKTQLQLEHLLLDLQMILNGINNYKNPKLTRMLTF |
| | N88S | KFYMPKKATELKHLQCLEEELKPLEEVLNLAQSKNFHLRPRD |
| | | LISSINVIVLELKGSETTFMCEYADETATIVEFLNRWITFCQ |
| | | SIISTLT |
| SEQ ID NO: 8 | human IL-2 | APTSSSTKKTQLQLEHLLLDLQMILNGINNYKNPKLTRMLTF |
| | C125S | KFYMPKKATELKHLQCLEEELKPLEEVLNLAQSKNFHLRPRD |
| | | LISNINVIVLELKGSETTFMCEYADETATIVEFLNRWITFSQ |
| | | SIISTLT |
| SEQ ID NO: 9 | human IL-2 | APTSSSTKKTQLQLEHLLLDLQMILNGINNYKNPKLTRMLTF |
| | N88D + C125S | KFYMPKKATELKHLQCLEEELKPLEEVLNLAQSKNFHLRPRD |
| | | LISDINVIVLELKGSETTFMCEYADETATIVEFLNRWITFSQ |
| | | SIISTLT |
| SEQ ID NO: 10 | human IL-2 | APTSSSTKKTQLQLEHLLLDLQMILNGINNYKNPKLTRMLTF |
| | N88R + C125S | KFYMPKKATELKHLQCLEEELKPLEEVLNLAQSKNFHLRPRD |
| | | LISRINVIVLELKGSETTFMCEYADETATIVEFLNRWITFSQ |
| | | SIISTLT |
| SEQ ID NO: 11 | human IL-2 | APTSSSTKKTQLQLEHLLLDLQMILNGINNYKNPKLTRMLTF |
| | N88S + C125S | KFYMPKKATELKHLQCLEEELKPLEEVLNLAQSKNFHLRPRD |
| | | LISSINVIVLELKGSETTFMCEYADETATIVEFLNRWITFSQ |
| | | SIISTLT |
| SEQ ID NO: 12 | human IL-2 | APTSSSTKKTQLQLEHLLLDLQMILNGINNYKNPKLTRMLTF |
| | V69A + | KFYMPKKATELKHLQCLEEELKPLEEALNLAQSKNFHLRPRD |
| | N88D + C125S | LISDINVIVLELKGSETTFMCEYADETATIVEFLNRWITFSQ |
| | | SIISTLT |
| SEQ ID NO: 13 | human IL-2 | APTSSSTKKTQLQLEHLLLDLQMILNGINNYKNPKLTRMLTF |
| | V69A + N88R + | KFYMPKKATELKHLQCLEEELKPLEEALNLAQSKNFHLRPRD |
| | C125S | LISRINVIVLELKGSETTFMCEYADETATIVEFLNRWITFSQ |
| | | SIISTLT |
| SEQ ID NO: 14 | human IL-2 | APTSSSTKKTQLQLEHLLLDLQMILNGINNYKNPKLTRMLTF |
| | V69A + | KFYMPKKATELKHLQCLEEELKPLEEALNLAQSKNFHLRPRD |
| | N88S + C125S | LISSINVIVLELKGSETTFMCEYADETATIVEFLNRWITFSQ |
| | | SIISTLT |
| SEQ ID NO: 15 | human IL-2 | APTSSSTKKTQLQLEHLLLDLQMILNGINNYKNPKLTRMLTF |
| | Q74P + | KFYMPKKATELKHLQCLEEELKPLEEVLNLAPSKNFHLRPRD |
| | N88D + C125S | LISDINVIVLELKGSETTFMCEYADETATIVEFLNRWITFSQ |
| | | SIISTLT |
| SEQ ID NO: 16 | human IL-2 | APTSSSTKKTQLQLEHLLLDLQMILNGINNYKNPKLTRMLTF |
| | Q74P + | KFYMPKKATELKHLQCLEEELKPLEEVLNLAPSKNFHLRPRD |
| | N88D + C125S | LISDINVIVLELKGSETTFMCEYADETATIVEFLNRWITFSQ |
| | | SIISTLT |
| SEQ ID NO: 17 | human IL-2 | APTSSSTKKTQLQLEHLLLDLQMILNGINNYKNPKLTRMLTF |
| | Q74P + | KFYMPKKATELKHLQCLEEELKPLEEVLNLAPSKNFHLRPRD |
| | N88D + C125S | LISDINVIVLELKGSETTFMCEYADETATIVEFLNRWITFSQ |
| | | SIISTLT |
| SEQ ID NO: 18 | human IL-2 | APTSSSTKKTQLQLEHLLLDLQMILNGINNYKNPKLTRMLTF |
| | V69A + Q74P + | KFYMPKKATELKHLQCLEEELKPLEEALNLAPSKNFHLRPRD |
| | N88D + C125S | LISDINVIVLELKGSETTFMCEYADETATIVEFLNRWITFSQ |
| | | SIISTLT |
| SEQ ID NO: 19 | human IL-2 | APTSSSTKKTQLQLEHLLLDLQMILNGINNYKNPKLTRMLTF |
| | V69A + Q74P + | KFYMPKKATELKHLQCLEEELKPLEEALNLAPSKNFHLRPRD |
| | N88R + C125S | LISRINVIVLELKGSETTFMCEYADETATIVEFLNRWITFSQ |
| | | SIISTLT |
| SEQ ID NO: 20 | human IL-2 | APTSSSTKKTQLQLEHLLLDLQMILNGINNYKNPKLTRMLTF |
| | V69A + Q74P + | KFYMPKKATELKHLQCLEEELKPLEEALNLAPSKNFHLRPRD |
| | N88S + C125S | LISSINVIVLELKGSETTFMCEYADETATIVEFLNRWITFSQ |
| | | SIISTLT |

TABLE 3
Sequences of IL-33 variants

| | | |
|---|---|---|
| SEQ ID NO: 21 | truncated wild | SITGISPITEYLASLSTYNDQSITFALEDESYEIYVEDLKKD |
| | type human IL- | EKKDKVLLSYYESQHPSNESGDGVDGKMLMVTLSPTKDFWLH |
| | 33 | ANNKEHSVELHKCEKPLPDQAFFVLHNMHSNCVSFECKTDPG |
| | | VFIGVKDNHLALIKVDSSENLCTENILFKLSET |
| SEQ ID NO: 22 | truncated N60S | SITGISPITEYLASLSTYNDQSITFALEDESYEIYVEDLKKD |
| | human IL-33 | EKKDKVLLSYYESQHPSSESGDGVDGKMLMVTLSPTKDFWLH |
| | | ANNKEHSVELHKCEKPLPDQAFFVLHNMHSNCVSFECKTDPG |
| | | VFIGVKDNHLALIKVDSSENLCTENILFKLSET |
| SEQ ID NO: 23 | truncated | SITGISPITEYLASLSTYNDQSITFALEDESYEIYVEDLKKD |
| | C116F human | EKKDKVLLSYYESQHPSNESGDGVDGKMLMVTLSPTKDFWLH |
| | IL-33 | ANNKEHSVELHKCEKPLPDQAFFVLHNMHSNFVSFECKTDPG |
| | | VFIGVKDNHLALIKVDSSENLCTENILFKLSET |
| SEQ ID NO: 24 | truncated C97G | SITGISPITEYLASLSTYNDQSITFALEDESYEIYVEDLKKD |
| | human IL-33 | EKKDKVLLSYYESQHPSNESGDGVDGKMLMVTLSPTKDFWLH |
| | | ANNKEHSVELHKGEKPLPDQAFFVLHNMHSNCVSFECKTDPG |
| | | VFIGVKDNHLALIKVDSSENLCTENILFKLSET |
| SEQ ID NO: 25 | truncated | SITGISPITEYLASLSTYNDQSITFALEDESYEIYVEDLKKD |
| | N60S + C116F | EKKDKVLLSYYESQHPSSESGDGVDGKMLMVTLSPTKDFWLH |
| | human IL-33 | ANNKEHSVELHKCEKPLPDQAFFVLHNMHSNFVSFECKTDPG |
| | | VFIGVKDNHLALIKVDSSENLCTENILFKLSET |
| SEQ ID NO: 26 | truncated | SITGISPITEYLASLSTYNDQSITFALEDESYEIYVEDLKKD |
| | N60S + C97G | EKKDKVLLSYYESQHPSSESGDGVDGKMLMVTLSPTKDFWLH |
| | human IL-33 | ANNKEHSVELHKGEKPLPDQAFFVLHNMHSNCVSFECKTDPG |
| | | VFIGVKDNHLALIKVDSSENLCTENILFKLSET |
| SEQ ID NO: 27 | truncated | SITGISPITEYLASLSTYNDQSITFALEDESYEIYVEDLKKD |
| | C116F + C97G | EKKDKVLLSYYESQHPSNESGDGVDGKMLMVTLSPTKDFWLH |
| | human IL-33 | ANNKEHSVELHKGEKPLPDQAFFVLHNMHSNFVSFECKTDPG |
| | | VFIGVKDNHLALIKVDSSENLCTENILFKLSET |
| SEQ ID NO: 28 | truncated | SITGISPITEYLASLSTYNDQSITFALEDESYEIYVEDLKKD |
| | N60S + C116F + | EKKDKVLLSYYESQHPSSESGDGVDGKMLMVTLSPTKDFWLH |
| | C97G human | ANNKEHSVELHKGEKPLPDQAFFVLHNMHSNFVSFECKTDPG |
| | IL-33 | VFIGVKDNHLALIKVDSSENLCTENILFKLSET |
| SEQ ID NO: 78 | truncated | SITGISPITEYLASLSTYNDQSITFALEDESYEIYVEDLKKD |
| | N60S + C116F + | EKKDKVLLSYYESQHPSSESGDGVDGKMLMVTLSPTKDFWLH |
| | C97G + C148G | ANNKEHSVELHKGEKPLPDQAFFVLHNMHSNFVSFECKTDPG |
| | human IL-33 | VFIGVKDNHLALIKVDSSENLGTENILFKLSET |
| SEQ ID NO: 79 | truncated | SITGISPITEYLASLSTYNDQSITFALEDESYEIYVEDLKKD |
| | N60S + C116F + | EKKDKVLLSYYESQHPSSESGDGVDGKMLMVTLSPTKDFWLH |
| | C97G + C121S + | ANNKEHSVELHKGEKPLPDQAFFVLHNMHSNFVSFESKTDPG |
| | C148G human | VFIGVKDNHLALIKVDSSENLGTENILFKLSET |
| | IL-33 | |

TABLE 4
Sequences of Linkers

| | | |
|---|---|---|
| SEQ ID NO: 29 | linker sequence | GGGGS |
| SEQ ID NO: 30 | linker sequence | GGGGSGGGGSGGGGS |
| SEQ ID NO: 31 | linker sequence | GGSGGGS |
| SEQ ID NO: 32 | linker sequence | EAAAK |
| SEQ ID NO: 33 | linker sequence | GGGGGGGG |
| SEQ ID NO: 34 | linker sequence | GSGSGSGSGS |
| SEQ ID NO: 35 | linker sequence | GSGSGSGSGSGSGSGSGSGS |
| SEQ ID NO: 36 | linker sequence | GGSGGSGGS |
| SEQ ID NO: 37 | linker sequence | GSGGS |
| SEQ ID NO: 38 | linker sequence | GSSGS |
| SEQ ID NO: 39 | linker sequence | GSAT |
| SEQ ID NO: 40 | linker sequence | SSSSGSSSSG |
| SEQ ID NO: 41 | linker sequence | LEGSGQGPGSGQGSGSPGSGQG |
| SEQ ID NO: 42 | linker sequence | GGGGSEAAAK |
| SEQ ID NO: 43 | linker sequence | GGGGSEAAAKGGGGSEAAAK |
| SEQ ID NO: 44 | linker sequence | EGKSSGSGSESKST |
| SEQ ID NO: 45 | linker sequence | GGGGSLVPRGSGGGGS |
| SEQ ID NO: 46 | linker sequence | KESGSVSSEQLAQFRSLD |
| SEQ ID NO: 47 | linker sequence | GGGSEGGGSEGGGSEGGG |
| SEQ ID NO: 48 | linker sequence | PAPAP |
| SEQ ID NO: 49 | linker sequence | AEAAAKEAAAKA |
| SEQ ID NO: 50 | linker sequence | AEAAAKEAAAKEAAAKEAAAKALEAEAAAKEAAAKEAAAKEA |
| | | AAKA |
| SEQ ID NO: 51 | CTP peptide | KAPPPS*LPSPS*RLPGPS*DTPILPQ |
| SEQ ID NO: 52 | signal peptide | MGWSCIILFLVATATGVHS |
| SEQ ID NO: 53 | Flag tag | DYKDDDDK |

TABLE 5
Sequences of Bifunctional Fusion Proteins

| | | |
|---|---|---|
| SEQ ID NO: 54 | IL233 Fusion | MGWSCIILFLVATATGVHSAPTSSSTKKTQLQLEHLLLDLQMI |
| | protein (SLT1) | LNGINNYKNPKLTRMLTFKFYMPKKATELKHLQCLEEELKPLE |
| | IL233 WT | EVLNLAQSKNFHLRPRDLISNINVIVLELKGSETTFMCEYADE |
| | (signal peptide | TATIVEFLNRWITFCQSIISTLTGGGGSGGGGSGGGGSSITGI |
| | underlined; WT | SPITEYLASLSTYNDQSITFALEDESYEIYVEDLKKDEKKDKV |
| | IL-2 | LLSYYESQHPSNESGDGVDGKMLMVTLSPTKDFWLHANNKEHS |
| | polypeptide in | VELHKCEKPLPDQAFFVLHNMHSNCVSFECKTDPGVFIGVKDN |
| | italics (N88); | HLALIKVDSSENLCTENILFKLSET |
| | peptide linker in | |
| | bold italics; WT | |
| | IL-33 | |
| | polypeptide | |
| | N60, C97, | |
| | C116, C121, | |
| | C148) | |
| SEQ ID NO: 55 | IL233 Fusion | MGWSCIILFLVATATGVHSAPTSSSTKKTQLQLEHLLLDLQMI |
| | protein (SLT2) | LNGINNYKNPKLTRMLTFKFYMPKKATELKHLQCLEEELKPLE |
| | N88D/WT N60, | EVLNLAQSKNFHLRPRDLISDINVIVLELKGSETTFMCEYADE |
| | C97, C116, | TATIVEFLNRWITFCQSIISTLTGGGGSGGGGSGGGGSSITGI |
| | C121, C148 | SPITEYLASLSTYNDQSITFALEDESYEIYVEDLKKDEKKDKV |
| | | LLSYYESQHPSNESGDGVDGKMLMVTLSPTKDFWLHANNKEHS |
| | | VELHKCEKPLPDQAFFVLHNMHSNCVSFECKTDPGVFIGVKDN |
| | | HLALIKVDSSENLCTENILFKLSET |
| SEQ ID NO: 56 | IL233 Fusion | MGWSCIILFLVATATGVHSAPTSSSTKKTQLQLEHLLLDLQMI |
| | protein (SLT3) | LNGINNYKNPKLTRMLTFKFYMPKKATELKHLQCLEEELKPLE |
| | N88D/N60S | EVLNLAQSKNFHLRPRDLISDINVIVLELKGSETTFMCEYADE |
| | | TATIVEFLNRWITFCQSIISTLTGGGGSGGGGSGGGGSSITGI |
| | | SPITEYLASLSTYNDQSITFALEDESYEIYVEDLKKDEKKDKV |
| | | LLSYYESQHPSSESGDGVDGKMLMVTLSPTKDFWLHANNKEHS |
| | | VELHKCEKPLPDQAFFVLHNMHSNCVSFECKTDPGVFIGVKDN |
| | | HLALIKVDSSENLCTENILFKLSET |
| SEQ ID NO: 57 | IL233 Fusion | MGWSCIILFLVATATGVHSAPTSSSTKKTQLQLEHLLLDLQMI |
| | protein (SLT4) | LNGINNYKNPKLTRMLTFKFYMPKKATELKHLQCLEEELKPLE |
| | N88D/C116F | EVLNLAQSKNFHLRPRDLISDINVIVLELKGSETTFMCEYADE |
| | | TATIVEFLNRWITFCQSIISTLTGGGGSGGGGSGGGGSSITGI |
| | | SPITEYLASLSTYNDQSITFALEDESYEIYVEDLKKDEKKDKV |
| | | LLSYYESQHPSNESGDGVDGKMLMVTLSPTKDFWLHANNKEHS |
| | | VELHKCEKPLPDQAFFVLHNMHSNFVSFECKTDPGVFIGVKDN |
| | | HLALIKVDSSENLCTENILFKLSET |
| SEQ ID NO: 58 | IL233 Fusion | MGWSCIILFLVATATGVHSAPTSSSTKKTQLQLEHLLLDLQMI |
| | protein (SLT5) | LNGINNYKNPKLTRMLTFKFYMPKKATELKHLQCLEEELKPLE |
| | N88D/C97G | EVLNLAQSKNFHLRPRDLISDINVIVLELKGSETTFMCEYADE |
| | | TATIVEFLNRWITFCQSIISTLTGGGGSGGGGSGGGGSSITGI |
| | | SPITEYLASLSTYNDQSITFALEDESYEIYVEDLKKDEKKDKV |
| | | LLSYYESQHPSNESGDGVDGKMLMVTLSPTKDFWLHANNKEHS |
| | | VELHKGEKPLPDQAFFVLHNMHSNCVSFECKTDPGVFIGVKDN |
| | | HLALIKVDSSENLCTENILFKLSET |
| SEQ ID NO: 59 | IL233 Fusion | MGWSCIILFLVATATGVHSAPTSSSTKKTQLQLEHLLLDLQMI |
| | protein (SLT6) | LNGINNYKNPKLTRMLTFKFYMPKKATELKHLQCLEEELKPLE |
| | N88D/N60S + | EVLNLAQSKNFHLRPRDLISDINVIVLELKGSETTFMCEYADE |
| | C116F + C97G | TATIVEFLNRWITFCQSIISTLTGGGGSGGGGSGGGGSSITGI |
| | | SPITEYLASLSTYNDQSITFALEDESYEIYVEDLKKDEKKDKV |
| | | LLSYYESQHPSSESGDGVDGKMLMVTLSPTKDFWLHANNKEHS |
| | | VELHKGEKPLPDQAFFVLHNMHSNFVSFECKTDPGVFIGVKDN |
| | | HLALIKVDSSENLCTENILFKLSET |
| SEQ ID NO: 60 | IL233 Fusion | MGWSCIILFLVATATGVHSAPTSSSTKKTQLQLEHLLLDLQMI |
| | protein (SLT7) | LNGINNYKNPKLTRMLTFKFYMPKKATELKHLQCLEEELKPLE |
| | N88D/C116F+ | EVLNLAQSKNFHLRPRDLISDINVIVLELKGSETTFMCEYADE |
| | C97G | TATIVEFLNRWITFCQSIISTLTGGGGSGGGGSGGGGSSITGI |
| | | SPITEYLASLSTYNDQSITFALEDESYEIYVEDLKKDEKKDKV |
| | | LLSYYESQHPSNESGDGVDGKMLMVTLSPTKDFWLHANNKEHS |
| | | VELHKGEKPLPDQAFFVLHNMHSNFVSFECKTDPGVFIGVKDN |
| | | HLALIKVDSSENLCTENILFKLSET |
| SEQ ID NO: 61 | IL233 Fusion | MGWSCIILFLVATATGVHSAPTSSSTKKTQLQLEHLLLDLQMI |
| | protein (SLT8) | LNGINNYKNPKLTRMLTFKFYMPKKATELKHLQCLEEELKPLE |
| | N88D + C125S/ | EVLNLAQSKNFHLRPRDLISDINVIVLELKGSETTFMCEYADE |
| | C116F + C97G | TATIVEFLNRWITFSQSIISTLTGGGGSGGGGSGGGGSSITGI |
| | | SPITEYLASLSTYNDQSITFALEDESYEIYVEDLKKDEKKDKV |
| | | LLSYYESQHPSNESGDGVDGKMLMVTLSPTKDFWLHANNKEHS |
| | | VELHKGEKPLPDQAFFVLHNMHSNFVSFECKTDPGVFIGVKDN |
| | | HLALIKVDSSENLCTENILFKLSET |
| SEQ ID NO: 62 | IL233 Fusion | MGWSCIILFLVATATGVHSAPTSSSTKKTQLQLEHLLLDLQMI |
| | protein (SLT9) | LNGINNYKNPKLTRMLTFKFYMPKKATELKHLQCLEEELKPLE |
| | N88D + C125S/ | EVLNLAQSKNFHLRPRDLISDINVIVLELKGSETTFMCEYADE |
| | C116F | TATIVEFLNRWITFSQSIISTLTGGGGSGGGGSGGGGSSITGI |
| | | SPITEYLASLSTYNDQSITFALEDESYEIYVEDLKKDEKKDKV |
| | | LLSYYESQHPSNESGDGVDGKMLMVTLSPTKDFWLHANNKEHS |
| | | VELHKCEKPLPDQAFFVLHNMHSNFVSFECKTDPGVFIGVKDN |
| | | HLALIKVDSSENLCTENILFKLSET |
| SEQ ID NO: 63 | IL233 Fusion | MGWSCIILFLVATATGVHSAPTSSSTKKTQLQLEHLLLDLQMI |
| | protein (SLT10) | LNGINNYKNPKLTRMLTFKFYMPKKATELKHLQCLEEELKPLE |
| | N88D + C125S/ | EVLNLAQSKNFHLRPRDLISDINVIVLELKGSETTFMCEYADE |
| | C116F/Fc- | TATIVEFLNRWITFSQSIISTLTGGGGSGGGGSGGGGSSITGI |
| | K77A | SPITEYLASLSTYNDQSITFALEDESYEIYVEDLKKDEKKDKV |
| | | LLSYYESQHPSNESGDGVDGKMLMVTLSPTKDFWLHANNKEHS |
| | | VELHKCEKPLPDQAFFVLHNMHSNFVSFECKTDPGVFIGVKDN |
| | | HLALIKVDSSENLCTENILFKLSETGGGGSGGGGSGGGGSDKT |
| | | HTCPPCPAPELLGGPSVFLFPPKPKDTLMISRTPEVTCVVVDV |
| | | SHEDPEVKFNWYVDGVEVHNAKTKPREEQYASTYRVVSVLTVL |
| | | HQDWLNGKEYKCKVSNKALPAPIEKTISKAKGQPREPQVYTLP |
| | | PSRDELTKNQVSLTCLVKGFYPSDIAVEWESNGQPENNYKTTP |
| | | PVLDSDGSFFLYSKLTVDKSRWQQGNVFSCSVMHEALHNHYTQ |
| | | KSLSLSPG |
| SEQ ID NO: 64 | IL233 Fusion | MGWSCIILFLVATATGVHSAPTSSSTKKTQLQLEHLLLDLQMI |
| | protein (SLT11) | LNGINNYKNPKLTRMLTFKFYMPKKATELKHLQCLEEELKPLE |
| | N88 + C125/C116F | EVLNLAQSKNFHLRPRDLISNINVIVLELKGSETTFMCEYADE |
| | | TATIVEFLNRWITFCQSIISTLTGGGGSGGGGSGGGGSSITGI |
| | | SPITEYLASLSTYNDQSITFALEDESYEIYVEDLKKDEKKDKV |
| | | LLSYYESQHPSNESGDGVDGKMLMVTLSPTKDFWLHANNKEHS |
| | | VELHKCEKPLPDQAFFVLHNMHSNFVSFECKTDPGVFIGVKDN |
| | | HLALIKVDSSENLCTENILFKLSET |
| SEQ ID NO: 65 | IL233 Fusion | MGWSCIILFLVATATGVHSAPTSSSTKKTQLQLEHLLLDLQMI |
| | protein (SLT12) | LNGINNYKNPKLTRMLTFKFYMPKKATELKHLQCLEEELKPLE |
| | C125S/C116F/ | EVLNLAQSKNFHLRPRDLISNINVIVLELKGSETTFMCEYADE |
| | Fc-K77A | TATIVEFLNRWITFSQSIISTLTGGGGSGGGGSGGGGSSITGI |
| | | SPITEYLASLSTYNDQSITFALEDESYEIYVEDLKKDEKKDKV |
| | | LLSYYESQHPSNESGDGVDGKMLMVTLSPTKDFWLHANNKEHS |
| | | VELHKCEKPLPDQAFFVLHNMHSNFVSFECKTDPGVFIGVKDN |
| | | HLALIKVDSSENLCTENILFKLSETGGGGSGGGGSGGGGSDKT |
| | | HTCPPCPAPELLGGPSVFLFPPKPKDTLMISRTPEVTCVVVDV |
| | | SHEDPEVKFNWYVDGVEVHNAKTKPREEQYASTYRVVSVLTVL |
| | | HQDWLNGKEYKCKVSNKALPAPIEKTISKAKGQPREPQVYTLP |
| | | PSRDELTKNQVSLTCLVKGFYPSDIAVEWESNGQPENNYKTTP |
| | | PVLDSDGSFFLYSKLTVDKSRWQQGNVFSCSVMHEALHNHYTQ |
| | | KSLSLSPG |
| SEQ ID NO: 66 | IL233 Fusion | MGWSCIILFLVATATGVHSDKTHTCPPCPAPELLGGPSVFLFP |
| | protein (SLT13) | PKPKDTLMISRTPEVTCVVVDVSHEDPEVKFNWYVDGVEVHNA |
| | Fc-K77A, | KTKPREEQYASTYRVVSVLTVLHQDWLNGKEYKCKVSNKALPA |
| | N88 + C125S/ | PIEKTISKAKGQPREPQVYTLPPSRDELTKNQVSLTCLVKGFY |
| | C116F | PSDIAVEWESNGQPENNYKTTPPVLDSDGSFFLYSKLTVDKSR |
| | | WQQGNVFSCSVMHEALHNHYTQKSLSLSPGKGGGGSGGGGSGG |
| | | GGSAPTSSSTKKTQLQLEHLLLDLQMILNGINNYKNPKLTRML |
| | | TFKFYMPKKATELKHLQCLEEELKPLEEVLNLAQSKNFHLRPR |
| | | DLISNINVIVLELKGSETTFMCEYADETATIVEFLNRWITFSQ |
| | | SIISTLTGGGGSGGGGSGGGGSSITGISPITEYLASLSTYNDQ |
| | | SITFALEDESYEIYVEDLKKDEKKDKVLLSYYESQHPSNESGD |
| | | GVDGKMLMVTLSPTKDFWLHANNKEHSVELHKCEKPLPDQAFF |
| | | VLHNMHSNFVSFECKTDPGVFIGVKDNHLALIKVDSSENLCTE |
| | | NILFKLSET |
| SEQ ID NO: 67 | IL233 Fusion | MGWSCIILFLVATATGVHSDKTHTCPPCPAPELLGGPSVFLFP |
| | protein (SLT14) | PKPKDTLMISRTPEVTCVVVDVSHEDPEVKFNWYVDGVEVHNA |
| | Fc- | KTKPREEQYASTYRVVSVLTVLHQDWLNGKEYKCKVSNKALPA |
| | K77/N88D + | PIEKTISKAKGQPREPQVYTLPPSRDELTKNQVSLTCLVKGFY |
| | C125S/C116F | PSDIAVEWESNGQPENNYKTTPPVLDSDGSFFLYSKLTVDKSR |
| | | WQQGNVFSCSVMHEALHNHYTQKSLSLSPGKGGGGSGGGGSGG |
| | | GGSAPTSSSTKKTQLQLEHLLLDLQMILNGINNYKNPKLTRML |
| | | TFKFYMPKKATELKHLQCLEEELKPLEEVLNLAQSKNFHLRPR |
| | | DLISDINVIVLELKGSETTFMCEYADETATIVEFLNRWITFSQ |
| | | SIISTLTGGGGSGGGGSGGGGSSITGISPITEYLASLSTYNDQ |
| | | SITFALEDESYEIYVEDLKKDEKKDKVLLSYYESQHPSNESGD |
| | | GVDGKMLMVTLSPTKDFWLHANNKEHSVELHKGEKPLPDQAFF |
| | | VLHNMHSNFVSFECKTDPGVFIGVKDNHLALIKVDSSENLCTE |
| | | NILFKLSET |
| SEQ ID NO: 68 | IL233 Fusion | MGWSCIILFLVATATGVHSAPTSSSTKKTQLQLEHLLLDLQMI |
| | protein (SLT15) | LNGINNYKNPKLTRMLTFKFYMPKKATELKHLQCLEEELKPLE |
| | V69A, Q74P, | EALNLAPSKNFHLRPRDLISDINVIVLELKGSETTFMCEYADE |
| | N88D + C125S/ | TATIVEFLNRWITFSQSIISTLTGGGGSGGGGSGGGGSSITGI |
| | C116F | SPITEYLASLSTYNDQSITFALEDESYEIYVEDLKKDEKKDKV |
| | | LLSYYESQHPSNESGDGVDGKMLMVTLSPTKDFWLHANNKEHS |
| | | VELHKCEKPLPDQAFFVLHNMHSNFVSFECKTDPGVFIGVKDN |
| | | HLALIKVDSSENLCTENILFKLSET |
| SEQ ID NO: 69 | IL233 Fusion | MGWSCIILFLVATATGVHSAPTSSSTKKTQLQLEHLLLDLQMI |
| | protein (SLT16) | LNGINNYKNPKLTRMLTFKFYMPKKATELKHLQCLEEELKPLE |
| | V69A, Q74P, | EALNLAPSKNFHLRPRDLISDINVIVLELKGSETTFMCEYADE |
| | N88D + C125S/ | TATIVEFLNRWITFSQSIISTLTGGGGSGGGGSGGGGSSITGI |
| | C97G + C116F | SPITEYLASLSTYNDQSITFALEDESYEIYVEDLKKDEKKDKV |
| | | LLSYYESQHPSNESGDGVDGKMLMVTLSPTKDFWLHANNKEHS |
| | | VELHKGEKPLPDQAFFVLHNMHSNFVSFECKTDPGVFIGVKDN |
| | | HLALIKVDSSENLCTENILFKLSET |
| SEQ ID NO: 70 | IL233 Fusion | MGWSCIILFLVATATGVHSAPTSSSTKKTQLQLEHLLLDLQMI |
| | protein (SLT17) | LNGINNYKNPKLTRMLTFKFYMPKKATELKHLQCLEEELKPLE |
| | V69A, Q74P, | EALNLAPSKNFHLRPRDLISDINVIVLELKGSETTFMCEYADE |
| | N88D + C125/ | TATIVEFLNRWITFCQSIISTLTGGGGSGGGGSGGGGSSITGI |
| | C97 + C116F | SPITEYLASLSTYNDQSITFALEDESYEIYVEDLKKDEKKDKV |
| | | LLSYYESQHPSNESGDGVDGKMLMVTLSPTKDFWLHANNKEHS |
| | | VELHKCEKPLPDQAFFVLHNMHSNFVSFECKTDPGVFIGVKDN |
| | | HLALIKVDSSENLCTENILFKLSET |
| SEQ ID NO: 71 | IL233 Fusion | MGWSCIILFLVATATGVHSDYKDDDDKAPTSSSTKKTQLQLEH |
| | protein (SLT18: | LLLDLQMILNGINNYKNPKLTRMLTFKFYMPKKATELKHLQCL |
| | SLT8 flag) | EEELKPLEEVLNLAQSKNFHLRPRDLISDINVIVLELKGSETT |
| | N88D + C125S/ | FMCEYADETATIVEFLNRWITFSQSIISTLTGGGGSGGGGSGG |
| | C116F + C97G | GGSSITGISPITEYLASLSTYNDQSITFALEDESYEIYVEDLK |
| | Flag | KDEKKDKVLLSYYESQHPSNESGDGVDGKMLMVTLSPTKDFWL |
| | | HANNKEHSVELHKGEKPLPDQAFFVLHNMHSNFVSFECKTDPG |
| | | VFIGVKDNHLALIKVDSSENLCTENILFKLSET |
| SEQ ID NO: 72 | IL233 Fusion | MGWSCIILFLVATATGVHSDYKDDDDKAPTSSSTKKTQLQLEH |
| | protein (SLT19: | LLLDLQMILNGINNYKNPKLTRMLTFKFYMPKKATELKHLQCL |
| | SLT9 flag) | EEELKPLEEVLNLAQSKNFHLRPRDLISDINVIVLELKGSETT |
| | N88D + C125S/ | FMCEYADETATIVEFLNRWITFSQSIISTLTGGGGSGGGGSGG |
| | C116F flag | GGSSITGISPITEYLASLSTYNDQSITFALEDESYEIYVEDLK |
| | | KDEKKDKVLLSYYESQHPSNESGDGVDGKMLMVTLSPTKDFWL |
| | | HANNKEHSVELHKCEKPLPDQAFFVLHNMHSNFVSFECKTDPG |
| | | VFIGVKDNHLALIKVDSSENLCTENILFKLSET |
| SEQ ID NO: 73 | IL233 Fusion | MGWSCIILFLVATATGVHSDYKDDDDKAPTSSSTKKTQLQLEH |
| | protein (SLT20: | LLLDLQMILNGINNYKNPKLTRMLTFKFYMPKKATELKHLQCL |
| | SLT15 flag) | EEELKPLEEALNLAPSKNFHLRPRDLISDINVIVLELKGSETT |
| | V69A, Q74P, | FMCEYADETATIVEFLNRWITFSQSIISTLTGGGGSGGGGSGG |
| | N88D + C125S/ | GGSSITGISPITEYLASLSTYNDQSITFALEDESYEIYVEDLK |
| | C116F | KDEKKDKVLLSYYESQHPSNESGDGVDGKMLMVTLSPTKDFWL |
| | Flag | HANNKEHSVELHKCEKPLPDQAFFVLHNMHSNFVSFECKTDPG |
| | | VFIGVKDNHLALIKVDSSENLCTENILFKLSET |
| SEQ ID NO: 74 | IL233 Fusion | MGWSCIILFLVATATGVHSDYKDDDDKAPTSSSTKKTQLQLEH |
| | protein (SLT21: | LLLDLQMILNGINNYKNPKLTRMLTFKFYMPKKATELKHLQCL |
| | SLT16 flag) | EEELKPLEEALNLAPSKNFHLRPRDLISDINVIVLELKGSETT |
| | V69A, Q74P, | FMCEYADETATIVEFLNRWITFSQSIISTLTGGGGSGGGGSGG |
| | N88D + C125S/ | GGSSITGISPITEYLASLSTYNDQSITFALEDESYEIYVEDLK |
| | C97G + C116F | KDEKKDKVLLSYYESQHPSNESGDGVDGKMLMVTLSPTKDEWL |
| | Flag | HANNKEHSVELHKGEKPLPDQAFFVLHNMHSNFVSFECKTDPG |
| | | VFIGVKDNHLALIKVDSSENLCTENILFKLSET |
| SEQ ID NO: 75 | IL233 Fusion | MGWSCIILFLVATATGVHSDYKDDDDKAPTSSSTKKTQLQLEH |
| | protein (SLT22: | LLLDLQMILNGINNYKNPKLTRMLTFKFYMPKKATELKHLQCL |
| | SLT17 flag) | EEELKPLEEALNLAPSKNFHLRPRDLISDINVIVLELKGSETT |
| | V69A, Q74P, | FMCEYADETATIVEFLNRWITFCQSIISTLTGGGGSGGGGSGG |
| | N88D + C125/ | GGSSITGISPITEYLASLSTYNDQSITFALEDESYEIYVEDLK |
| | C97 + C116F Flag | KDEKKDKVLLSYYESQHPSNESGDGVDGKMLMVTLSPTKDFWL |
| | | HANNKEHSVELHKCEKPLPDQAFFVLHNMHSNFVSFECKTDPG |
| | | VFIGVKDNHLALIKVDSSENLCTENILFKLSET |
| SEQ ID NO. 80 | IL233 Fusion | MGWSCIILFLVATATGVHSAPTSSSTKKTQLQLEHLLLDLQMI |
| | protein (SLT27) | LNGINNYKNPKLTRMLTFKFYMPKKATELKHLQCLEEELKPLE |
| | | EVLNLAQSKNFHLRPRDLISDINVIVLELKGSETTFMCEYADE |
| | | TATIVEFLNRWITFSQSIISTLTGGGGSGGGGSGGGGSSITGI |
| | | SPITEYLASLSTYNDQSITFALEDESYEIYVEDLKKDEKKDKV |
| | | LLSYYESQHPSNESGDGVDGKMLMVTLSPTKDFWLHANNKEHS |
| | | VELHKGEKPLPDQAFFVLHNMHSNFVSFECKTDPGVFIGVKDN |
| | | HLALIKVDSSENLGTENILEKLSET |
| SEQ ID NO. 81 | IL233 Fusion | MGWSCIILFLVATATGVHSAPTSSSTKKTQLQLEHLLLDLQMI |
| | protein (SLT28 | LNGINNYKNPKLTRMLTFKFYMPKKATELKHLQCLEEELKPLE |
| | | EVLNLAQSKNFHLRPRDLISDINVIVLELKGSETTFMCEYADE |
| | | TATIVEFLNRWITFSQSIISTLTGGGGSGGGGSGGGGSSITGI |
| | | SPITEYLASLSTYNDQSITFALEDESYEIYVEDLKKDEKKDKV |
| | | LLSYYESQHPSNESGDGVDGKMLMVTLSPTKDFWLHANNKEHS |
| | | VELHKGEKPLPDQAFFVLHNMHSNFVSFESKTDPGVFIGVKDN |
| | | HLALIKVDSSENLGTENILFKLSET |

Signal peptides are underlined.
Flag tag is in bold font.
IL-2 polypeptide is in italics
Linker is bold and italics.
IL-33 polypeptide is in regular font.
Amino acid mutations are bold and underlined (WT residues at the same position are in bold).
Half-life extension moiety (e.g., Fc sequences) are in bold.

TABLE 6
Exemplary IL-33 Variant and WT with Signal Peptide and Tag

| | | |
|---|---|---|
| SEQ ID NO: 76 | IL-33 WT (C97, | MGWSCIILFLVATATGVHSDYKDDDDKSITGISPITEYLASLS |
| | C116, C121, | TYNDQSITFALEDESYEIYVEDLKKDEKKDKVLLSYYESQHPS |
| | C148) with | NESGDGVDGKMLMVTLSPTKDFWLHANNKEHSVELHKCEKPLP |
| | signal peptide | DQAFFVLHNMHSNCVSFECKTDPGVFIGVKDNHLALIKVDSSE |
| | and Flag tag | NLCTENILFKLSET |
| SEQ ID NO: 77 | Exemplary IL- | MGWSCIILFLVATATGVHSDYKDDDDKSITGISPITEYLASLS |
| | 33 variant | TYNDQSITFALEDESYEIYVEDLKKDEKKDKVLLSYYESQHPS |
| | (C97G and | NESGDGVDGKMLMVTLSPTKDFWLHANNKEHSVELHKGEKPLP |
| | C116F, C121, | DQAFFVLHNMHSNFVSFECKTDPGVFIGVKDNHLALIKVDSSE |
| | C148) with | NLCTENILFKLSET |
| | signal peptide | |
| | and Flag tag | |
| SEQ ID NO: 82 | Exemplary IL- | MGWSCIILFLVATATGVHSDYKDDDDKSITGISPITEYLASLS |
| | 33 variant | TYNDQSITFALEDESYEIYVEDLKKDEKKDKVLLSYYESQHPS |
| | (C97G C116F, | NESGDGVDGKMLMVTLSPTKDFWLHANNKEHSVELHKGEKPLP |
| | C121, C148G) | DQAFFVLHNMHSNFVSFECKTDPGVFIGVKDNHLALIKVDSSE |
| | with signal | NLGTENILFKLSET |
| | peptide and Flag | |
| | tag | |
| SEQ ID NO: 83 | Exemplary IL- | MGWSCIILFLVATATGVHSDYKDDDDKSITGISPITEYLASLS |
| | 33 variant | TYNDQSITFALEDESYEIYVEDLKKDEKKDKVLLSYYESQHPS |
| | (C97G, C116F, | NESGDGVDGKMLMVTLSPTKDFWLHANNKEHSVELHKGEKPLP |
| | C121S and | DQAFFVLHNMHSNFVSFESKTDPGVFIGVKDNHLALIKVDSSE |
| | C148G) with | NLGTENILFKLSET |
| | signal peptide | |
| | and Flag tag | |

### Examples

### Example 1. Design and Construction of IL-33 and IL-2 Bifunctional Proteins

This example illustrates the design and construction of exemplary IL-33 and IL-2 bifunctional proteins.

Standard recombinant DNA techniques were used to manipulate DNA (Sambrook et al., Molecular cloning: A laboratory manual; Cold Spring Harbor Laboratory Press, Cold Spring Harbor, N.Y., 1989) and design constructs containing nucleotide sequences encoding IL-2 and IL-33 proteins. Sequencing of double stranded DNA was carried out to confirm the nucleotide sequence of the constructs.

Exemplary nucleotide residues encoding the WT IL-2 protein (SEQ ID NO: 1 or 2) were mutated to generate exemplary IL-2 variant constructs. Exemplary IL-2 variants include at least one amino acid substitution in relation to the wild-type IL-2 protein. Exemplary IL-2 variants are listed in Table 2.

For example, the IL-2 variant comprises one or more amino acid substitutions selected from in relation to the wild-type IL-2 selected from a group consisting of T3N, T3A, L12G, L12K, L12Q, L12S, Q13G, E15A, E15G, E15S, H16A, H16D, H16E, H16G, H16K, H16M, H16N, H16R, H16S, H16T, H16V, H16Y, L19A, L19D, L19E, L19G, L19N, L19R, L19S, L19T, L19V, D20A, D20E, D20H, D201, D20Y, D20F, D20G, D20T, D20W, M23R, V69A, Q47P, R81A, R81G, R81S, R81T, D84A, D84E, D84G, D84K, D841, D84M, D84Q D84R, D84S, D84T, S87R, N88A, N88D, N88R, N88E, N88I, N88F, N88G, N88M, N88R, N88S, N88V, N88W, V91D, V91E, V91K, V91G, V91N, V91R, V91S, 192K, I92R, E95G, C125A, C125S, Q1261, Q126L, and Q126F.

Exemplary IL-2 variants of the present invention, for example, comprise one or more amino acid substitutions selected from V69A, Q47P, N88D, N88S and C125S.

Exemplary nucleotide residues encoding the truncated WT IL-33 polypeptide (SEQ ID NO: 21) were mutated to generate exemplary IL-33 variant constructs. Exemplary IL-33 variants include at least one amino acid substitution in relation to the wild-type IL-33 protein selected from the group consisting of N60, C97, C116, C121, or C148 (positions numbered relative to truncated IL-33), and wherein the substitution at any one of the cysteines C97, C116, C121, or C148 is not a serine. Exemplary IL-33 variants also include IL-33 variants comprising an amino acid substitution at N60 and any one of C97, C116, C121, or C148. Exemplary IL-33 variants also include variants comprising an amino acid substitution at C116F and an additional amino acid at any one of C97, C121, or C148.

Included herein are IL-33 variants comprising at least one amino acid substitution selected from N60S, N60D, C97G, and C116F. Exemplary IL-33 variants comprise amino acid substitutions of C97G and C116F. Other exemplary IL-33 variants comprise amino acid substitutions of C97G, C116F and C148G; or C97G, C116F, C121S and C148G. Exemplary IL-33 variants are listed in Table 3.

Further, bifunctional fusion proteins were constructed, where the IL-2 polypeptide and IL-33 polypeptide were joined, for example, by a linker (e.g., a peptide linker).

### Example 2. Activity and Potency of a Bifunctional Protein Comprising IL-2 and IL-33 Variants

This example illustrates the activity and potency of an exemplary bifunctional protein comprising IL-2 and IL-33 variant polypeptides.

Briefly, IL-33 WT polypeptide (SEQ ID NO: 76, comprising signal sequence and flag tag), an IL-33 variant (SEQ ID NO: 77, comprising signal sequence and flag tag) and a bifunctional protein comprising an IL-2 variant and an IL-33 variant (SLT8) (SEQ ID NO: 61) were tested for activity in a reporter assay measured by NF-κB/AP-1− inducible secreted embryonic alkaline phosphatase (SEAP) reporter gene levels in an HEK-Blue™ IL-33 reporter cell line (InvivoGen), which provides an indication of downstream IL-33 signaling.

Further, IL-33 WT polypeptide (SEQ ID NO: 76, comprising signal sequence and flag tag), an IL-33 variant (SEQ ID NO: 77, comprising signal sequence and flag tag) and a bifunctional protein comprising an IL-2 variant and an IL-33 variant (SLT8) (SEQ ID NO: 61) were tested for activity in a reporter assay measured by STAT5-inducible secreted embryonic alkaline phosphatase (SEAP) reporter gene levels in an HEK-Blue™ IL-2 reporter cell line (InvivoGen), which provides an indication of downstream IL-2 signaling.

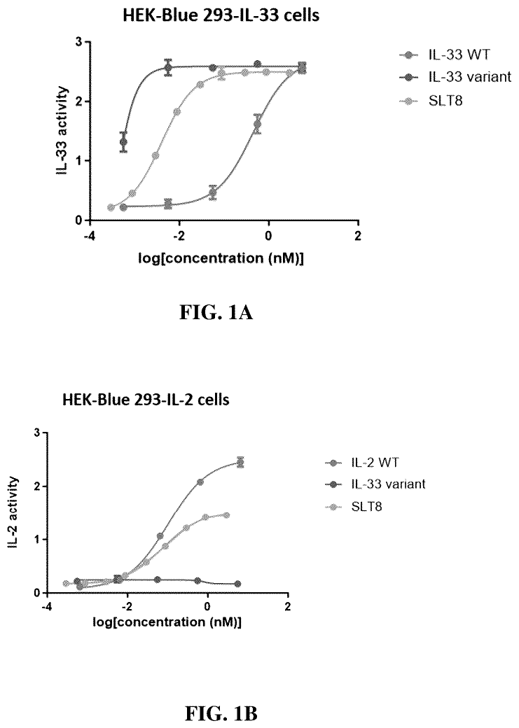

FIG. 1A is a graph showing potency of IL-33 WT, IL-33 variant and a bifunctional protein, SLT8 comprising IL-33 variant and an IL-2 polypeptide as measured by SEAP levels depicting IL-33 activity plotted relative to a log of the concentration of the respective protein in nM. From the graph, the EC50 was determined as depicted in Table 7 below.

FIG. 1B is a graph showing potency of IL-33 variant, IL-2 WT and a bifunctional protein, SLT8 comprising IL-33 variant and an IL-2 polypeptide as measured by SEAP levels depicting IL-2 activity plotted relative to a log of the concentration of IL-33 variant. From the graph, the EC50 was determined as depicted in Table 7 below.

TABLE 7
Relative Potency of IL-33 Variant, IL-33 WT, IL-2 WT
and an exemplary bifunctional fusion protein, SLT8

| Sample | EC50 (nM) |
|---|---|
| IL-33 WT (SEQ ID NO: 76) | 0.4509 |
| IL-33 variant (SEQ ID NO: 77) | 0.0002999 |
| Fusion of IL-2 and IL-33 variant (SLT8) | 0.004017 |
| (SEQ ID NO: 61) | |

The results showed that an exemplary IL-33 variant of the present disclosure (SEQ ID NO: 77) had lower EC50 indicating higher potency in cells relative to truncated IL-33 WT (SEQ ID NO: 76). A bifunctional fusion protein (SEQ ID NO: 61), SLT8 comprising an exemplary IL-33 variant and an IL-2 polypeptide also showed reduced EC50 relative to WT IL-33.

Overall, the results showed that IL-33 variants and bifunctional proteins comprising IL-33 variants and IL-2 had higher potency than truncated IL-33 WT.

### Example 3. Expression, Stability, Potency and Purity of Exemplary Bifunctional Proteins

This example illustrates expression and purity of bifunctional proteins comprising IL-2 and IL-33 variants. Potency is evaluated at various temperatures as a measure of stability.

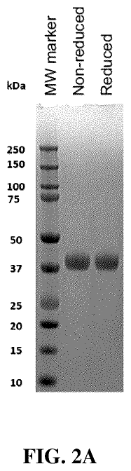

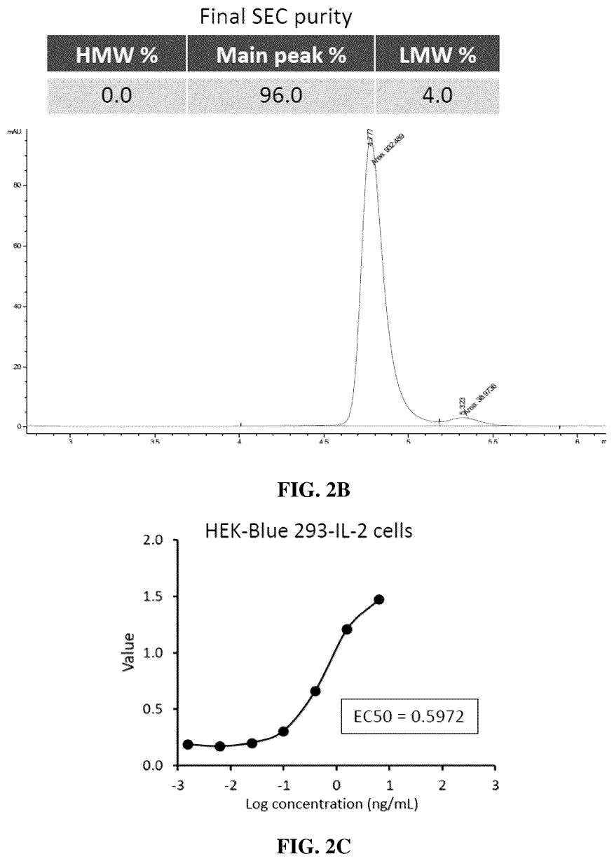

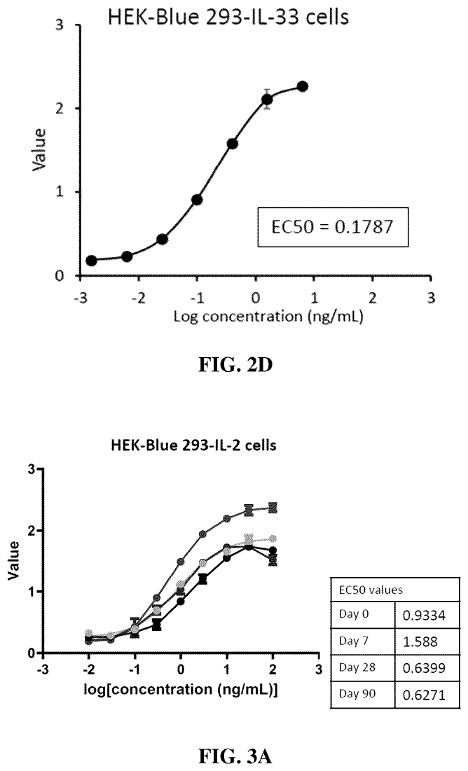

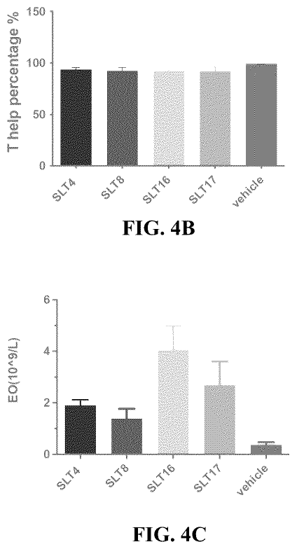

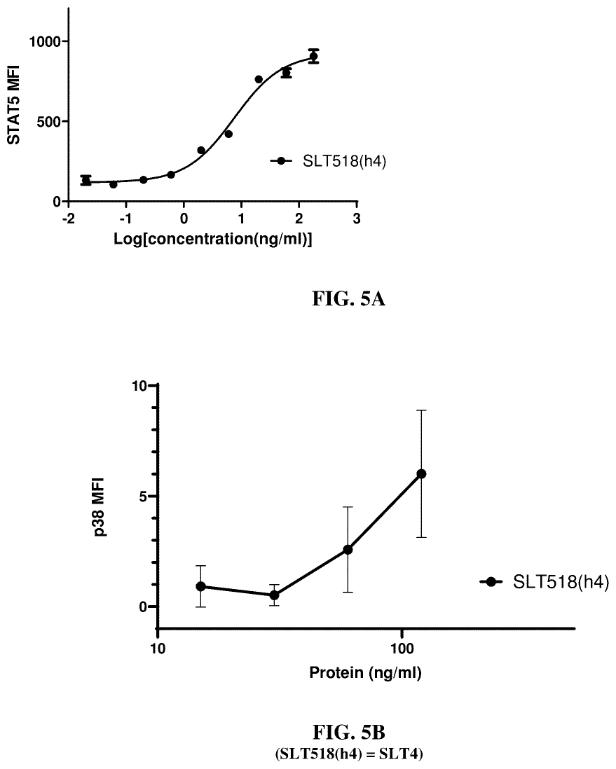

FIG. 4A is an SDS PAGE gel showing expression of an exemplary IL233 bifunctional protein, SLT8, in reducing and non-reducing conditions. The SLT8 protein purified from CHO cells showed greater than 95% purity. FIG. 2B is chromatogram of an exemplary bifunctional protein purified from bacteria (e.g., E. coli) showing a major peak of 96% purity, comprising no high molecular weight (HMW) species and about 4% low molecular weight (LMW) species. FIG. 2C is a graph showing potency of an exemplary bifunctional protein, SLT8 purified from E. coli as measured by SEAP levels plotted relative to a log of the concentration of the protein in a STAT5− inducible secreted embryonic alkaline phosphatase (SEAP) reporter gene levels in an HEK-Blue™ IL-2 reporter cell line (InvivoGen). From the graph, the EC50 was determined as 0.5972, indicative of high potency. FIG. 5D is a graph showing potency of an exemplary bifunctional protein, SLT8 purified from E. coli as measured by SEAP levels plotted relative to a log of the concentration of the protein in a NF-κB/AP-1− inducible secreted embryonic alkaline phosphatase (SEAP) reporter gene levels in an HEK-Blue™ IL-33 reporter cell line (InvivoGen). From the graph, the EC50 was determined as 0.1787, indicative of high potency.

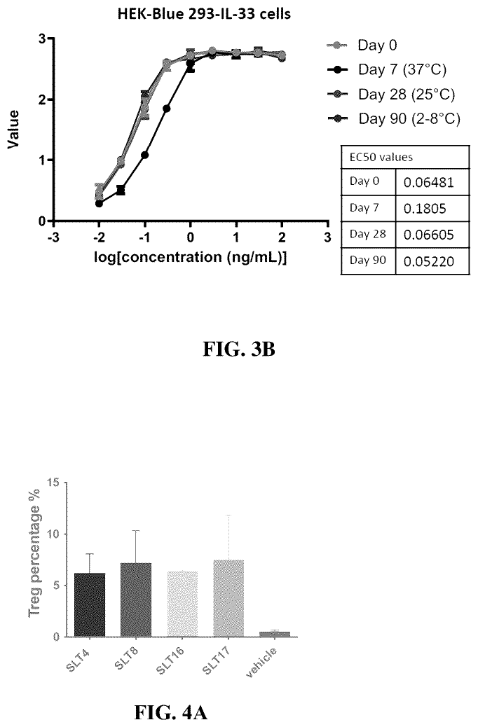

FIG. 3A shows graphs of potency of exemplary bifunctional protein, SLT8 as measured by SEAP levels plotted relative to a log of the concentration of the protein in a STAT5− inducible secreted embryonic alkaline phosphatase (SEAP) reporter gene levels (indicative of IL-2 signaling) in an HEK-Blue™ IL-2 reporter cell line (InvivoGen) at day 0, and upon storage until day 7 at 37° C., day 28 at 25° C. and day 90 at between 2-8° C. From the graph, the EC50 was determined as 0.9334 at day 0, 1.588 at day 7 upon storage at 37° C., 0.6399 at day 28 upon storage at 25° C. and 0.6271 at day 90 upon storage at between 2-8° C., indicative of maintenance of high potency and stability upon storage, for example, upon storage at 2-8° C. and 25° C. FIG. 3B shows graphs of potency of exemplary bifunctional protein, SLT8 as measured by SEAP levels plotted relative to a log of the concentration of the protein in a NF-κB/AP-1− inducible secreted embryonic alkaline phosphatase (SEAP) reporter gene levels (indicative of IL-33 signaling) in an HEK-Blue™ IL-33 reporter cell line (InvivoGen) at day 0, and upon storage until day 7 at 37° C., day 28 at 25° C. and day 90 at between 2-8° C. From the graph, the EC50 was determined as 0.06481 at day 0, 0.1805 at day 7 upon storage at 37° C., 0.06605 at day 28 upon storage at 25° C. and 0.05220 at day 90 upon storage at between 2-8° C., indicative of maintenance of high potency and stability upon storage, for example, upon storage at 2-8° C. and 25° C.

Overall, the results showed that SLT8 can be purified to a high degree of purity from both CHO cells and bacteria (e.g., E. coli), and that SLT8 is stable upon storage at day 7, day 28 and at day 90 at between 2-8° C. and 25° C.

### Example 4. Immune Cell Types in Human Cells Treated with Exemplary Bifunctional Proteins

This example illustrates the types of immune cells induced in human cells treated with bifunctional proteins comprising IL-2 and IL-33 variants. The relative proportion of regulatory T cells, helper T cells, and eosinophils are measured.

FIG. 4A is a bar graph showing percentage of regulatory T cells (Tregs) as measured by treatment of human cells with exemplary bifunctional proteins, SLT4, SLT8, SLT16, SLT17 relative to vehicle control, which was comparable in all the bifunctional proteins shown in this graph. FIG. 4B is a bar graph showing percentage of helper T cells as measured in exemplary bifunctional proteins, SLT4, SLT8, SLT16, SLT17 relative to vehicle control, which was comparable in all the bifunctional proteins shown in this graph. FIG. 4C is a bar graph showing percentage of eosinophils as measured in exemplary bifunctional proteins, SLT4, SLT8, SLT16, SLT17 relative to vehicle control. SLT4 and SLT8 showed a very low percentage of eosinophils, indicative of low immunogenicity.

FIG. 5A is a graph showing downstream IL-2 signaling in human PBMCs by measuring phosphorylated STAT5 levels of an exemplary bifunctional protein, SLT4. Whole blood cells from healthy donors were collected and stimulated with SLT4. The pSTAT5 levels were indicative of primarily IL-2 activity. FIG. 5B is a graph showing downstream IL-33 signaling in human PBMCs by measuring phosphorylated p38 levels indicative of activation of MAPK signaling of an exemplary bifunctional protein, SLT4. Whole blood cells from healthy donors were collected and stimulated with SLT4. The p38 levels were indicative of primarily IL-33 activity.

Overall, the results showed that Treg cells were preferentially induced by bifunctional proteins relative to vehicle control. SLT4 and SLT8 showed reduced eosinophil amounts indicative of low immunogenicity.

### Example 5. Dose Dependent ST2⁺ T_{reg} Proliferation in Fresh Human PBMCs

This example illustrates that ST2+ Treg proliferation is dose dependent in healthy human PBMC donors.

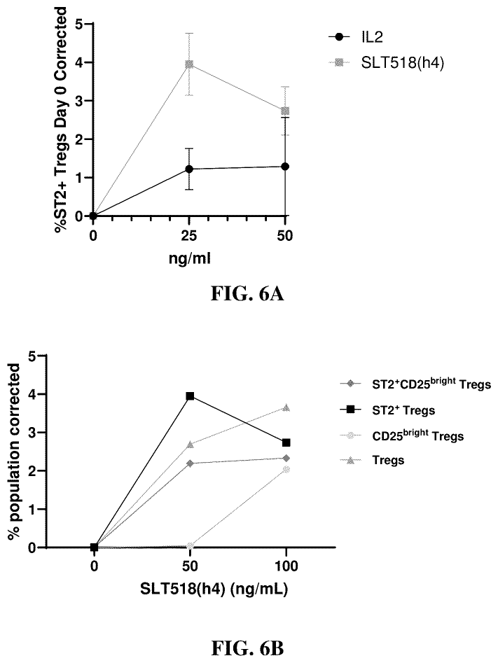

FIG. 6A is a graph showing percentage of ST2+ Tregs in fresh human PBMCs stimulated with an exemplary bifunctional protein, SLT4 or stimulated with IL-2 as a control. FIG. 6B is a graph showing induction of total Treg proliferation by SLT4 as well as the percentage of Treg subsets in SLT4 stimulated PBMCs, comprising mainly ST2+CD25 bright Tregs and ST2+ Tregs. CD25 bright Tregs were induced at higher concentrations of SLT4.

Overall, the results showed induction of ST2 and CD25 bright Tregs by the bifunctional proteins of the present invention, associated with low expression of eosinophils indicating low immunogenicity.

### Example 6. Regulatory T Cells in Synovial Fluid from Injured Knee

This example measured the proportion of Tregs, and specifically, ST2+ Tregs in synovial fluid from injured knee.

Briefly, leukocytes were isolated from fresh synovial fluid from injured knee and stained for ST2+ Tregs. Leukocytes were cultured for 24 hours in the presence of equimolar amounts of human IL-2 or an exemplary bifunctional protein, SLT4. Cells were analyzed by flow cytometry for the proportion and number of total and ST2+ Tregs.

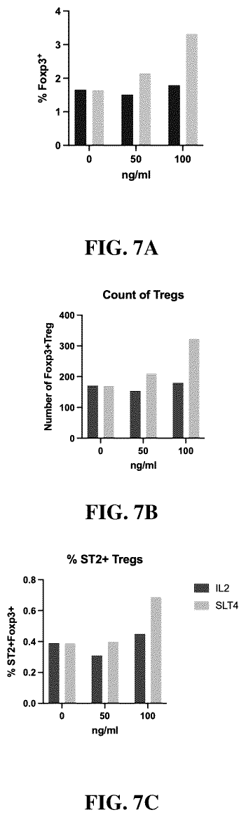

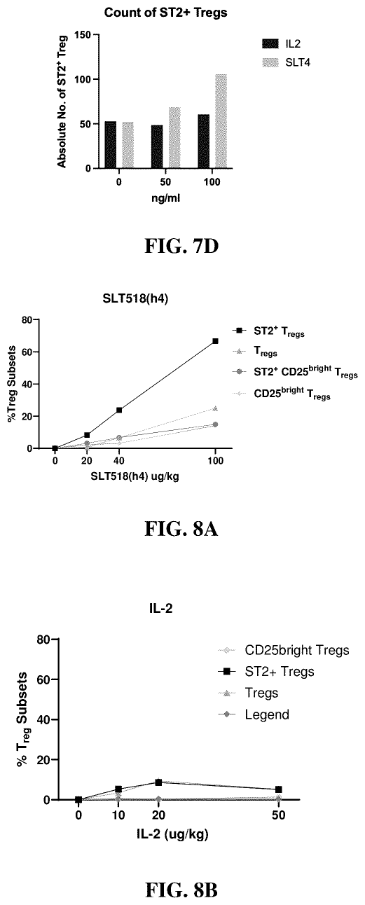

FIG. 7A-7D are bar graphs showing the percentage or absolute number of total Tregs and ST2+ Tregs in leukocytes isolated from fresh synovial fluid from injured knee.

FIG. 7A is a bar graph showing percentage of Fox3+ Tregs at various concentrations of SLT4 and IL-2 WT control ranging from 0 ng/ml, 50 ng/ml and 100 ng/ml. The percentage of total Tregs is increased in the SLT4 sample at both 50 ng/ml and 100 ng/ml concentrations. FIG. 7B is a bar graph showing total number of Fox3+ Tregs at various concentrations of SLT4 and IL-2 WT control ranging from 0 ng/ml, 50 ng/ml and 100 ng/ml. The number of total Tregs is increased in the SLT4 sample at both 50 and 100 ng/ml concentrations. FIG. 7C is a bar graph showing percentage of ST2+ Fox3+ Tregs at various concentrations of SLT4 and IL-2 WT control ranging from 0 ng/ml, 50 ng/ml and 100 ng/ml. The percentage of ST2+ Tregs is increased in the SLT4 sample at both 50 ng/ml and 100 ng/ml concentrations. FIG. 7D is a bar graph showing total number of ST2+ Fox3+ Tregs at various concentrations of SLT4 and IL-2 WT control ranging from 0 ng/ml, 50 ng/ml and 100 ng/ml. The number of total Tregs is increased in the SLT4 sample at both 50 ng/ml and 100 ng/ml concentrations.

Overall, the number of Tregs was increased in samples treated with bifunctional protein relative to IL-2 WT control.

### Example 7. IL233 Bifunctional Proteins Induce Expansion of Tissue Targeting T_{reg} Subsets in a Dose Dependent Manner

This example illustrates that all Treg subsets are induced in healthy non-human primates in a dose dependent manner. These include ST2+, ST3+CD25bright Tregs, and CD25bright Tregs as well as total Tregs.

FIG. 8A is a graph showing the percentage of Treg subsets at various concentrations of an exemplary bifunctional protein, SLT4 ranging from 0 ng/ml, 20 ng/ml, 40 ng/ml and 100 ng/ml in healthy non-human primates. The graph shows that tissue targeting Treg subsets are all expanded in a dose-dependent manner, including predominantly ST2+ Tregs. FIG. 8B is a graph showing the percentage of Treg subsets at various concentrations of IL-2 ranging from 0 ng/ml, 20 ng/ml, 40 ng/ml and 100 ng/ml in healthy non-human primates. The graph shows that IL-2 is not as effective at expanding tissue targeting Treg subsets, including ST2+ Tregs, as compared to SLT4.

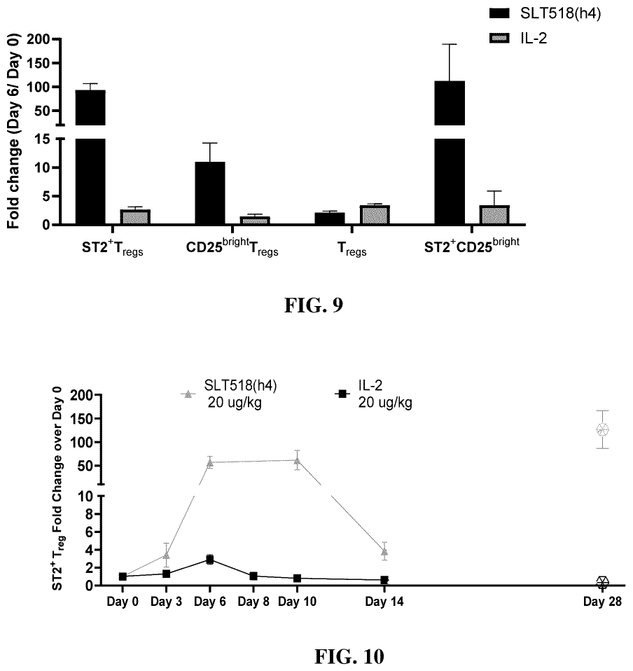

FIG. 9 is a bar graph showing fold change of Treg subsets on day 6 relative to day 0 at a low dose of 20 μg/kg in healthy non-human primates. Baseline levels of Treg cells stimulated by IL-2 treatment are shown for comparison. The graphs show that even at a low dose, an exemplary bifunctional protein, SLT4 shows induction of ST2+ and ST2+CD25 bright Tregs. In addition, CD25+ bright Tregs are also increased.

Overall, the percentage of Tregs subsets was increased in samples treated with bifunctional protein relative to IL-2 control.

### Example 8. ST2+ Tregs Induced by the Bifunctional Proteins Persist in Tissue Over a Long Duration

This example illustrates a time-course of ST2+ Treg cells induced after treatment with an exemplary bifunctional protein, SLT4 or IL-2 WT control from days 1-14 and on day 28 after treatment in non-human primates. ST2+T_{regs} in circulating blood was measured from days 1-14 and tissue targeted ST2+ cells were measured in inguinal lymph node tissue on day 28.

FIG. 10 is a graph showing fold change of ST2+ Treg cells relative to day 0 at a low dose of 20 μg/kg of SLT4 and IL-2 WT as a control in a time course from day 0 to day 14. Relative to IL-2 WT, the SLT4 sample showed a greater fold change induction of ST2+ Treg cells starting at day 0 and increasing until day 6, staying consistently high until 10, when it started decreasing and was approaching comparable levels, although still higher than IL-2 by day 14. On day 28, ST2+ Tregs were higher with SLT4 than IL-2 WT alone. The IL-2 control treatment leads to increased circulating ST2+ Treg cells but not as much as SLT4 treatment, while the SLT4 treatment leads to elevated levels of tissue-targeted ST2+ Tregs in inguinal lymph node.

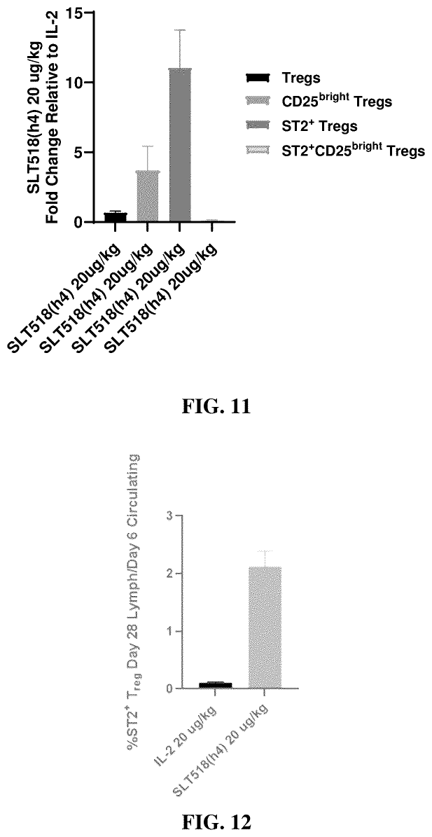

FIG. 11 is a bar graph showing fold change of total Treg and Treg subsets by SLT4 relative to IL-2 WT on day 28 in lymph. Total Tregs were increased by SLT4 relative to IL-2 control. The greatest induction was of ST2+ Treg cells, followed by CD25bright Tregs. ST2+CD25 bright cells induced by SLT4 on day 28 were comparable to IL-2 WT control.

FIG. 12 is a bar graph showing percentage of ST2+ Treg cells on day 28 in lymph relative to ST2+ circulating systemically on day 6 at a low dose of 20 μg/kg of SLT4 relative to an IL-2 WT control. The percentage of ST2+ Tregs in inguinal lymph node tissue remains much higher than ST2+ Tregs circulating in blood, indicative of tissue targeting and a sustained and durable expression and response.

Overall results show that ST2+ Tregs in inguinal lymph node tissue are higher in non-human primates treated with the bifunctional protein than IL-2 alone and that the tissue targeted ST2+ Tregs persist, leading to durable expression and response in tissue.

### Example 9. Bifunctional Protein Selectively Expands Treg Cells in Non-Human Primates

This example illustrates that an exemplary bifunctional protein, SLT4 selectively expands Treg cells in non-human primates.

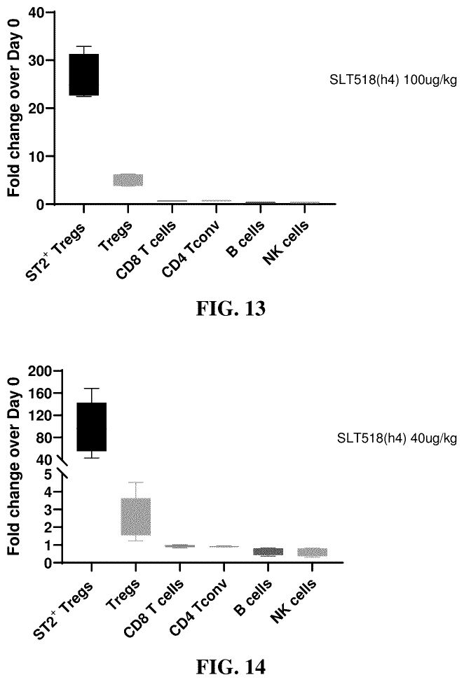

FIG. 13 is a bar graph showing fold change expansion of total Tregs and ST2+ Treg subsets relative to other immune cells, such as T effector cells (CD8, CD4), B cells and NK cells, over day 0 in non-human primates. The greatest expansion was of the ST2+ Treg cells at a dose of 100 μg/kg of SLT4, showing selective expansion of ST2+ Tregs in non-human primates.

FIG. 14 is a bar graph showing fold change expansion of total Tregs and ST2+ Treg subsets relative to other immune cells, such as T effector cells (CD8, CD4), B cells and NK cells, over day 0, in non-human primates. The greatest expansion was of the ST2+ Treg cells at a dose of 40 μg/kg of SLT4, showing selective expansion of ST2+ Tregs in non-human primates.

At both low and high doses of treatment, ST2+ Tregs are expanded the most of all immune cell types tested in non-human primates treated with an exemplary IL233 bifunctional protein of the present invention.

### Example 10. Ratio of Treg Cells to T Effector Cells is High in Non-Human Primates Treated with IL233 Bifunctional Protein

This example illustrates that treatment with bifunctional proteins increases the ratio of Treg to T effector cell, showing a high selectivity index for Tregs.

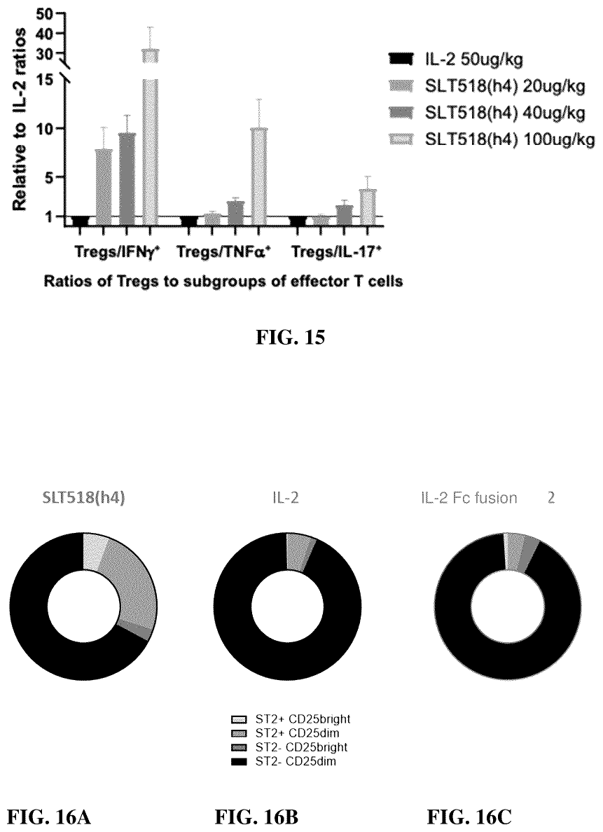

FIG. 15 is a bar graph of ratios of Tregs to subgroups of T effector cells, i.e., Tregs relative to FN, Tregs relative to TNFα and Tregs relative to IL-17, at 20 μg/kg, 40 μg/kg and 100 μg/kg of SLT4 in non-human primates. The ratios obtained with SLT4 are normalized to ratios obtained with 50 μg/kg of IL-2. The graph indicates selectivity of SLT4 to Tregs in non-human primates.

Overall, the results showed higher selectivity for Treg cells over T effector cells, in non-human primates treated with the IL233 bifunctional protein relative to an IL-2 WT control.

### Example 11. Bifunctional Protein is Better than IL-2 or IL-2 Fused to Fc in Equimolar Doses

This example illustrates that equimolar doses of IL233 bifunctional proteins increase Tregs to a greater proportion than an IL-2 control or a commercial IL-2 fused to Fc.

FIG. 16A-16C shows a comparison of Treg subsets upon SLT4 administration in non-human primates (FIG. 16A) relative to IL-2 (FIG. 16B) and IL-2 Fc fusion (FIG. 16C). SLT4 showed the largest expansion of ST2+ Treg cells followed by ST2+CD25bright and was more effective than IL-2 or IL-2 Fc fusion.

Overall, the results showed that IL-2 and IL-2 fused to Fe are less potent at expanding Tregs than a bifunctional protein comprising IL-2 and IL-33 variants. ST2+ Tregs and ST2+CD25bright were the largest subsets expanded.

### Example 12. Expression, Stability, Potency and Purity of Exemplary Bifunctional Protein, SLT27

This example illustrates expression and purity of bifunctional protein SLT27 comprising IL-2 and IL-33 variants. Potency was evaluated at various temperatures as a measure of stability.

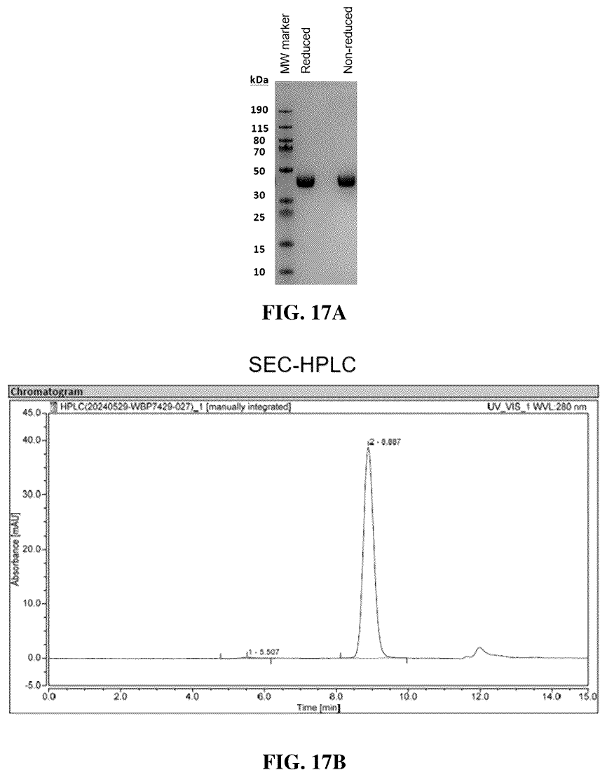

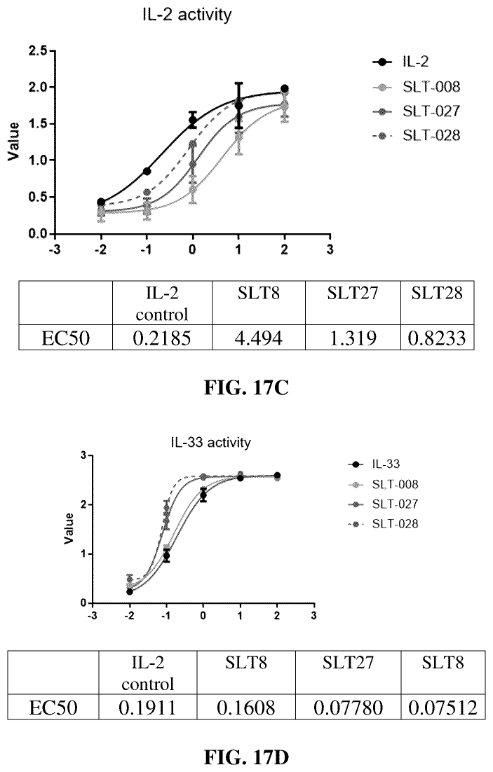

FIG. 17A is an SDS PAGE gel showing expression of an exemplary IL233 bifunctional protein, SLT-27, in reducing and non-reducing conditions. The SLT-27 protein purified from CHO cells showed greater than 99% purity. FIG. 17B is a chromatogram of an exemplary bifunctional protein purified from mammalian culture (e.g., CHO) showing a major peak of 99.45% purity, comprising minimal high molecular weight (HMW) species (about 0.55%) and no low molecular weight (LMW) species. FIG. 17C is a graph showing comparable potency of exemplary bifunctional proteins, SLT8, SLT27 and SLT28 purified from CHO as measured by SEAP levels plotted relative to a log of the concentration of the protein in a STAT5− inducible secreted embryonic alkaline phosphatase (SEAP) reporter gene levels in an HEK-Blue™ IL-2 reporter cell line (InvivoGen). From the graph, the EC50 was determined as 4.494 (SLT8), 1.319 (SLT27) and 0.8233 (SLT28), indicative of high potency. FIG. 17D is a graph showing comparable potency of exemplary bifunctional proteins, SLT8, SLT27 and SLT28 purified from CHO as measured by SEAP levels plotted relative to a log of the concentration of the protein in a NF-κB/AP-1− inducible secreted embryonic alkaline phosphatase (SEAP) reporter gene levels in an HEK-Blue™ IL-33 reporter cell line (InvivoGen). From the graph, the EC50 was determined as 0.1608 (SLT8), 0.07780 (SLT27) and 0.07512 (SLT28), indicative of high potency.

Overall, the results showed that SLT27 was purified to a high degree of purity from CHO cells and showed comparable in vitro potency for both IL-2 and IL-33 activity exhibited by the bifunctional proteins, SLT8, SLT27 and SLT28.

### Example 13. Induction of ST2+ and Total Tregs by Exemplary Bifunctional Proteins, SLT8 and SLT27

Mice were subcutaneously injected with SLT8 or SLT27 (3 mice per study) at two different doses of 10 μg/kg or 100 μg/kg on day 0, 1 and 2. Pharmacodynamic analyses were carried out at day 4, 6 and 10. Complete blood count for measuring total number of eosinophils was carried out at day 4, 6, 10. A PBS control was obtained on day 4 after injection.

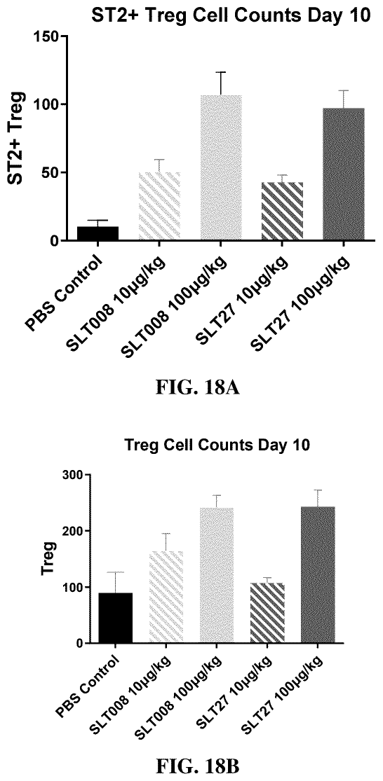

FIG. 18A is a bar graph showing the number of ST2+ Treg cells at day 10, measured from mice subcutaneously injected with a dose of 10 μg/kg or 100 μg/kg of exemplary bifunctional protein, SLT8 or SLT27 at days 0, 1 or 2. Both SLT8 and SLT27 showed a dose-dependent expansion of ST2+ Treg cells by about 5-10 fold. FIG. 18B is a bar graph showing the amount of total Treg cells at day 10, measured from mice subcutaneously injected with a dose of 10 μg/kg or 100 μg/kg of exemplary bifunctional protein, SLT8 or SLT27 at days 0, 1 or 2. Both SLT8 and SLT27 showed a dose-dependent expansion of total Treg cells by about 2-3 fold.

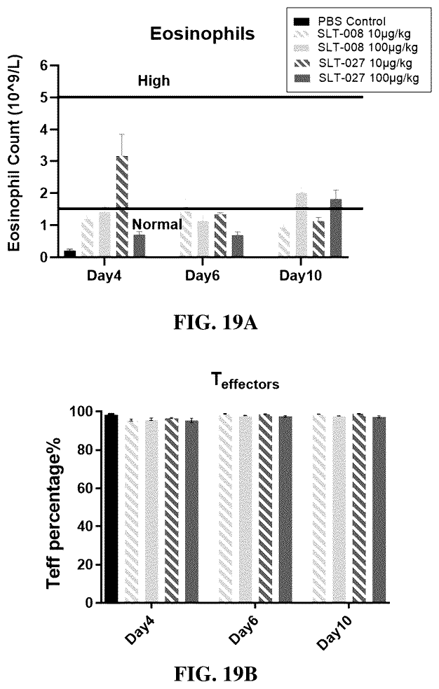

FIG. 19A is a bar graph of the number of eosinophils measured at days 4, 6 and 10 from mice subcutaneously injected with a dose of 10 μg/kg or 100 μg/kg of exemplary bifunctional protein, SLT8 or SLT27 at days 0, 1 or 2. SLT8 and SLT27 showed comparable amounts of eosinophils at the timepoints tested. A normal range for eosinophils is between 0-0.5×10\hat{ }9/L. Greater than 5×10\hat{ }9/L is considered high. Even with a moderate transient increase in eosinophils at day 4, total eosinophil levels remained low, indicative of low immunogenicity.

FIG. 19B is a bar graph of the percentage of T effector cells measured at days 4, 6 and 10 from mice subcutaneously injected with a dose of 10 μg/kg or 100 μg/kg of exemplary bifunctional protein, SLT8 or SLT27 at days 0, 1 or 2. SLT8 and SLT27 showed comparable amounts of T effector cells at the timepoints tested.

Overall, the results showed that SLT8 and SLT27 showed comparable expansion of ST2+ Treg and total Tregs across the timepoints and doses tested. The results also showed that selectivity of Tregs to eosinophils or T_{effector} cells was comparable between SLT8 and SLT27. Eosinophil levels remained low, indicative of low immunogenicity.

### Example 14. Stability of Exemplary Bifunctional SLT27 Protein at Different Storage Temperatures

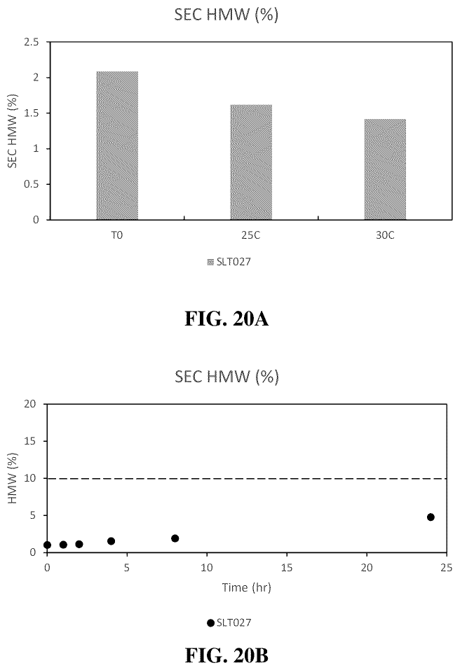

Short-term stability was measured after 2 weeks of storage of purified SLT27 at either 25° C. or 30° C. Percent high molecular weight aggregates is shown in Table 8. FIG. 20A is a bar graph showing aggregation as determined by percentage of high molecular weight products produced from exemplary bifunctional protein, SLT27 on day 0 and after storage for two weeks at either 25° C. or 30° C. From the graph, the percentage of high molecular weight products was determined as 2.09 at day 0, 1.62 upon storage at 25′C and 1.42 upon storage at 30° C., indicative of maintenance of stability upon storage.

TABLE 8
Percent High Molecular Weight Aggregates of
SLT27 at 25° C. and 30° C.

| SLT27 | T₀ | 25° C. | 30° C. |
|---|---|---|---|
| % HMW | 2.09 | 1.62 | 1.42 |

The results in FIG. 20A and Table 8 showed that SLT27 provided significant prevention of aggregation and was stable at 2 weeks with minimal change in high molecular weight aggregates upon storage at both 25° C. and 30° C. compared to the start of storage (To).

FIG. 20B shows a graph of percentage of high molecular weight products produced from storage of exemplary bifunctional protein, SLT27 at a temperature of 37° C. for 0, 1, 2, 4, 8 and 24 hours. Table 9 provides a quantitation of high molecular weight SLT27 aggregates upon storage at 37° C. for up to 24 hours.

TABLE 9
Percent High Molecular Weight Aggregates
and Fragments of SLT27 up to 24 hours

| SLT27 | T₀ | 1 hr | 2 hr | 4 hr | 8 hr | 24 hr |
|---|---|---|---|---|---|---|
| Conc (g/l) | 4.33 | 4.28 | 4.29 | 4.31 | 4.28 | 4.29 |
| SEC HMW (%) | 1.04 | 1.08 | 1.15 | 1.55 | 1.93 | 4.78 |
| NR CE-SDS | 0.57 | 0.61 | 0.61 | 0.61 | 0.68 | 0.77 |
| Fragment (%) | | | | | | |

The results in Table 9 and FIG. 20B showed that SLT27 provided significant prevention of aggregation. A less than 100 increase in HMW aggregates was observed over 8 hours by SEC HMW analysis. SLT27 showed less than 10% increase in HMW aggregates at 4 hours. SLT27 showed a low level of fragments with no significant increase observed in non-reducing CE-SDS fragment analysis. SLT27 did not show visible or measurable precipitation.

Overall, the results showed that SLT27 was stable upon storage and showed less than 10% increase in high molecular weight aggregates up to 24 hours at a higher temperature of 37° C. SLT27 was also stable at 2 weeks at both 25° C. and 30° C.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

What is claimed is:

1. A bifunctional protein comprising:
an IL-2 polypeptide, and
an IL-33 variant that has improved activity and/or manufacturability as compared to human WT IL-33,
wherein the IL-2 polypeptide is associated with the IL-33 variant, and wherein the bifunctional protein preferentially expands ST2+ Treg cells.

2. The bifunctional protein of claim 1, wherein the IL-2 polypeptide is linked to the IL-33 variant via a peptide linker or a chemical linker.

3. The bifunctional protein of claim 2, wherein the linker is a peptide linker comprising 3-30 amino acids.

4. A bifunctional protein comprising an IL-2 polypeptide and an IL-33 polypeptide, wherein the IL-2 polypeptide and the IL-33 polypeptide are associated with each other at a distance of between 1 nm to 10 nm.

5. A bifunctional protein comprising an IL-2 polypeptide and an IL-33 polypeptide, wherein the IL-2 polypeptide and the IL-33 polypeptide are associated with each other at a distance of between 10 Å to 100 Å.

6. The bifunctional protein of claim 4, wherein the distance is about 1.5 nm to 10 nm, 3.5 nm to 10 nm, 3.5 nm to 7 nm, 5 nm to 10 nm, or 5 nm to 7 nm.

7. The bifunctional protein of claim 5, wherein the distance is about 15 Å to 100 Å, 35 Å to 100 Å, 35 to 70 Å, 50 Å to 100 Å, or 50 Å to 70 Å.

8. The bifunctional protein of any one of claims 4-7, wherein the IL-2 polypeptide and the IL-33 polypeptide are linked by a linker.

9. The bifunctional protein of claim 8, wherein the linker is a peptide linker or a chemical linker.

10. A bifunctional protein comprising from the N-terminus to the C-terminus, an IL-2 polypeptide, a linker, and an IL-33 variant that has improved activity and/or manufacturability as compared to human WT IL-33, wherein the linker comprises 3-30 amino acids.

11. The bifunctional protein of any one of claims 1-10, wherein the IL-33 variant or polypeptide is a truncated IL-33 and at least one amino acid substitution at positions selected from N60, C97, C116, C121, and C148 according to the sequence of SEQ ID NO. 21.12. The bifunctional protein of claim 11, wherein the IL-33 variant or polypeptide comprises two, three, four or five amino acid substitutions selected from positions of N60, C97, C116, C121 and C148.

13. The bifunctional protein of claim 12, wherein the IL-33 variant or polypeptide comprises an amino acid substitution at position N60, wherein the substitution is N60S, N60T, N60Q, N60D, N60E or N60A.

14. The bifunctional protein of claim 12, wherein the IL-33 variant or polypeptide comprises an amino acid substitution at position C97, wherein the substitution is C97G, C97A or C97V.

15. The bifunctional protein of claim 12, wherein the IL-33 variant or polypeptide comprises an amino acid substitution at position C116, wherein the substitution is C116F, C116Y or C116A.

16. The bifunctional protein of claim 12, wherein the IL-33 variant or polypeptide comprises an amino acid substitution at position C121, wherein the substitution is C121S or C121A.

17. The bifunctional protein of claim 12, wherein the IL-33 variant or polypeptide comprises an amino acid substitution at position C148, wherein the substitution is C148G, C148S, C148Y, C148N or C148A.

18. The bifunctional protein of claim 12, wherein the IL-33 variant or polypeptide comprises the amino acid substitutions of N60S, C97G and C116F.

19. The bifunctional protein of claim 18, wherein the IL-33 variant or polypeptide further comprises the amino acid substitution C148G.

20. The bifunctional protein of claim 19, wherein the IL-33 variant or polypeptide further comprises the amino acid substitution C121S.

21. The bifunctional protein of any one of the preceding claims, wherein the IL-2 polypeptide comprises one or more amino acid substitutions selected from the group consisting of T3N, T3A, L12G, L12K, L12Q, L12S, Q13G, E15A, E15G, E15S, H16A, H16D, H16E, H16G, H16K, H16M, H16N, H16R, H16S, H16T, H16V, H16Y, L19A, L19D, L19E, L19G, L19N, L19R, L19S, L19T, L19V, D20A, D20E, D20H, D201, D20Y, D20F, D20G, D20T, D20W, M23R, V69A, Q47P, R81A, R81G, R81S, R81T, D84A, D84E, D84G, D84K, D841, D84M, D84Q D84R, D84S, D84T, S87R, N88A, N88D, N88R, N88E, N88I, N88F, N88G, N88M, N88R, N88S, N88V, N88W, V91D, V91E, V91K, V91G, V91N, V91R, V91S, 192K, 192R, E95G, C125A, C125S, Q1261, Q126L, and Q126F.

22. The bifunctional protein of claim 21, wherein the IL-2 polypeptide comprises one or more amino acid substitutions selected from the group consisting of V69A, Q47P, N88D, N88R and C125S.

23. The bifunctional protein of claim 21, wherein the IL-2 polypeptide comprises amino acid substitutions of N88D and C125S.

24. The bifunctional protein of any one of the preceding claims, wherein the bifunctional protein is capable of binding to CD25 and ST2 on the surface of a single cell.

25. The bifunctional protein of any one of the preceding claims, wherein the bifunctional protein is capable of binding to CD25 and ST2 on the surface of different cells.

26. The bifunctional protein of any one of the preceding claims, wherein the bifunctional protein expands circulating FoxP3⁺Treg cells, CD25^{bright} Treg cells, tissue targeting ST2+ Treg cells, CD25^{bright}ST2^{bright} Treg cells, Foxp3− Tr1 cells, and/or Foxp3− Th3 cells.

27. A fusion protein comprising,
an IL-2 polypeptide, and
an IL-33 polypeptide comprising amino acid substitutions of N60S, C97G, and C116F, according to the sequence of SEQ ID NO. 21,
wherein the IL-2 polypeptide is linked to the IL-33 polypeptide via a linker.

28. The fusion protein of claim 27, wherein the IL-33 polypeptide further comprises an amino acid substitution C148G.

29. The fusion protein of claim 28, wherein the IL-33 polypeptide further comprises an amino acid substitution C121S.

30. A fusion protein comprising,
an IL-2 polypeptide, and
an IL-33 polypeptide comprising the amino acid substitution C116F, C116Y or C116A according to the sequence of SEQ ID NO. 21,
wherein the IL-2 polypeptide is linked to the IL-33 polypeptide via a linker.

31. The fusion protein of claim 30, wherein the IL-33 polypeptide further comprises amino acid substitutions at positions N60, C97, C121 and/or C148.

32. The fusion protein of claim 31, wherein the IL-33 polypeptide comprises one or more amino acid substitutions N60S, N60D, C97G, C116F, C121S or C148G.

33. The fusion protein of claim 31, wherein the IL-33 polypeptide comprises the amino acid substitutions N60S, C97G, and C116F.

34. The fusion protein of claim 33, wherein the IL-33 polypeptide further comprises the amino acid substitution C148G.

35. The fusion protein of claim 34, wherein the IL-33 polypeptide further comprises the amino acid substitution C121S.

36. The fusion protein of any one of claims 30-35, wherein the IL-2 polypeptide is an IL-2 variant biased to IL2Rα.

37. The fusion protein of claim 30, wherein the IL-2 variant comprises one or more amino acid substitutions selected from T3N, T3A, L12G, L12K, L 2Q, L12S, Q13G, E15A, E15G, E15S, H16A, H16D, H16E, H16G, H16K, H16M, H16N, H16R, H16S, H16T, H16V, H16Y, L19A, L19D, L19E, L19G, L19N, L19R, L19S, L19T, L19V, D20A, D20E, D20H, D201, D20Y, D20F, D20G, D20T, D20W, M23R, V69A, Q47P, R81A, R81G, R81S, R81T, D84A, D84E, D84G, D84K, D841, D84M, D84Q D84R, D84S, D84T, S87R, N88A, N88D, N88R, N88E, N88I, N88F, N88G, N88M, N88R, N88S, N88V, N88W, V91D, V91E, V91K, V91G, V91N, V91R, V91S, 192K, I92R, E95G, C125A, C125S Q1261, Q126L, and Q126F.

38. The fusion protein of claim 37, wherein the IL-2 variant comprises one or more amino acid substitutions selected from V69A, Q47P, N88D and C125S.

39. The fusion protein of claim 38, wherein the IL-2 variant comprises the amino acid substitution N88D.

40. The fusion protein of claim 39, wherein the IL-2 variant comprises the amino acid substitutions of N88D and C125S.

41. The fusion protein of claim 38, wherein the IL-2 variant comprises the amino acid substitutions of V69A, Q47P, N88D and C125S.

42. The bifunctional protein or the fusion protein of any one of the preceding claims, wherein the IL-2 variant or polypeptide comprises an amino acid sequence selected from SEQ ID NOs: 3-20, and wherein the IL-33 variant or polypeptide comprises an amino acid sequence selected from SEQ ID NOs: 22-28.

43. The bifunctional protein or the fusion protein of any one of the preceding claims, wherein the peptide linker is selected from GGGGS (SEQ ID NO: 29; n repeats of SEQ ID NO: 29, n=1-5), GGGGSGGGGSGGGGS (SEQ ID NO: 31), EAAAK (SEQ ID NO: 32; n repeats of SEQ ID NO: 32, n=1-3), GGGGGGGG (SEQ ID NO: 33), GSGSGSGSGS (SEQ ID NO: 34), GSGSGSGSGSGSGSGSGSGS (SEQ ID NO: 35), GGSGGSGGS (SEQ ID NO: 36), GSGGS (SEQ ID NO: 37), GSSGS (SEQ ID NO: 38), a SEG-linker, a GSAT (SEQ ID NO: 39), SSSSGSSSSG (SEQ ID NO: 40), a flexible 22 amino acid linker LEGSGQGPGSGQGSGSPGSGQG (SEQ ID NO: 41), and GGGGSEAAAK (SEQ ID NO: 42, n=1-2), EGKSSGSGSESKST (SEQ ID NO: 44), GGGGSLVPRGSGGGGS (SEQ ID NO: 45), KESGSVSSEQLAQFRSLD (SEQ ID NO: 46), GGGSEGGGSEGGGSEGGG (SEQ ID NO: 47) or rigid peptide linkers PAPAP (SEQ ID NO: 48), (Ala-Pro)ₙ, AEAAAKEAAAKA (SEQ ID NO: 49), AEAAAKEAAAKEAAAKEAAAKALEAEAAAKEAAAKEAAAKEAAAKA (SEQ ID NO: 50).

44. The bifunctional protein or the fusion protein of claim 43, wherein the linker comprises GGGGSGGGGSGGGGS (SEQ ID NO: 31).

45. The bifunctional protein or the fusion protein of any one of the preceding claims, wherein the protein further comprises a signal peptide.

46. The bifunctional protein or the fusion protein of any one of claims 26-45, wherein the IL-33 variant or polypeptide has increased activity and/or manufacturability as compared to human WT IL-33.

47. A bifunctional protein comprising an IL-2 variant associated with an IL-33 variant, wherein the IL-2 variant has amino acid substitution N88D and the IL-33 variant has amino acid substitution C116F.

48. The bifunctional protein of claim 47, wherein the IL-2 variant has amino acid substitutions N88D and C125S and the IL-33 variant has amino acid substitutions C97G, C116F, C121S and/or C148G.

49. The bifunctional protein of claim 47 or 48, wherein the IL-2 variant is linked to the IL-33 variant by a linker.

50. The bifunctional protein of claim 49, wherein the linker is a peptide linker or a chemical linker.

51. The bifunctional protein of any one of claims 42-50, wherein the bifunctional protein, upon administration in vivo, results in eosinophil level at less than 5-fold, less than 4 fold, less than 3 fold or less than 2 fold as compared to the level before the treatment.

52. A bifunctional fusion protein comprising an amino acid sequence having at least 85%, 90%, 95%, 96%, 97%, 98%, or 100% identity to any one of the sequences of SEQ ID NO: 54 to SEQ ID NO:75 and SEQ ID NO: 80 to SEQ ID NO: 81.

53. A bifunctional fusion protein comprising an amino acid sequence having at least 90% identity to SEQ ID NO: 57.

54. The bifunctional fusion protein of claim 53, comprising an amino acid sequence having at least 95% identity to SEQ ID NO: 57.

55. The bifunctional fusion protein of claim 54, comprising an amino acid sequence having 100% identity to SEQ ID NO: 57.

56. A bifunctional fusion protein comprising an amino acid sequence having at least 90% identity to SEQ ID NO: 61.

57. The bifunctional fusion protein of claim 56, comprising an amino acid sequence having at least 95% identity to SEQ ID NO: 61.

58. The bifunctional fusion protein of claim 57, comprising an amino acid sequence having 100% identity to SEQ ID NO: 61.

59. A bifunctional fusion protein comprising an amino acid sequence having at least 90% identity to SEQ ID NO: 69.

60. The bifunctional fusion protein of claim 59, comprising an amino acid sequence having at least 95% identity to SEQ ID NO: 69.

61. The bifunctional fusion protein of claim 60, comprising an amino acid sequence having 100% identity to SEQ ID NO: 69.

62. A bifunctional fusion protein comprising an amino acid sequence having at least 90% identity to SEQ ID NO: 70.

63. The bifunctional fusion protein of claim 62, comprising an amino acid sequence having at least 95% identity to SEQ ID NO: 70.

64. The bifunctional fusion protein of claim 63, comprising an amino acid sequence having 100% identity to SEQ ID NO: 70.65. A bifunctional fusion protein comprising an amino acid sequence having at least 90% identity to SEQ ID NO: 80.

66. The bifunctional fusion protein of claim 65, comprising an amino acid sequence having at least 95% identity to SEQ ID NO: 80.

67. The bifunctional fusion protein of claim 66, comprising an amino acid sequence having 100% identity to SEQ ID NO: 80.

68. A bifunctional fusion protein comprising an amino acid sequence having at least 90% identity to SEQ ID NO: 81.

69. The bifunctional fusion protein of claim 68, comprising an amino acid sequence having at least 95% identity to SEQ ID NO: 81.

70. The bifunctional fusion protein of claim 69, comprising an amino acid sequence having 100% identity to SEQ ID NO: 81.

71. A polynucleotide encoding the bifunctional protein or the fusion protein of any one of the preceding claims.

72. A composition comprising the bifunctional protein or the fusion protein of any one of claims 1-70.

73. A pharmaceutical composition comprising the bifunctional protein or the fusion protein of any one of claims 1-70, and at least a pharmaceutically acceptable carrier.

74. A vector comprising the polynucleotide of claim 71.

75. The vector of claim 74, wherein the vector is a viral vector or a non-viral vector.

76. An engineered cell comprising the polynucleotide of claim 71 or the vector of claim 74 or 75.

77. The engineered cell of claim 76, wherein the cell is a mammalian cell.

78. The engineered cell of claim 77, wherein the cell is a bacterial cell, yeast cell or insect cell.

79. A method for modulating regulatory T cells (Treg) in a subject comprising administering to the subject the bifunctional protein or the fusion protein of any one of claims 1-70, or the composition of any one of claim 72 or 73.

80. The method of claim 79, wherein the method expands ST2+ Treg cells, CD25^{bright} Treg cells and/or circulating Foxp3+ Treg and Foxp3-negative Tr1 or Th3 cells.

81. The method of claim 80, wherein the ST2+ Treg cells comprise ST2⁺CD25^{bright}double positive Tregs.

82. The method of claim 81, wherein a proportion of ST2+ Treg and/or ST2⁺CD25^{bright}double positive Tregs traffic to a tissue at the site of disease.

83. The method of claim 82, wherein the method increases immune tolerance in the tissue microenvironment.

84. A method for promoting local immune homeostasis in a tissue microenvironment to support Treg durability in a subject comprising administering to the subject the bifunctional protein or the fusion protein of any one of claims 1-70, or the composition of any one of claims 72-73.

85. The method of claim 84, wherein the fusion protein upregulates ST2+ Treg cells in the tissue microenvironment.

86. The method of claim 85, wherein ST2+ Treg cells comprise ST2⁺CD25^{bright} double positive Treg cells.

87. The method of any one of claims 84-86, wherein the fusion protein further activates M2 macrophages, eosinophils, type 2 innate lymphoid cells (ILC2), and/or Th17+ Treg cells in the tissue microenvironment.

88. The method of any one of claims 83-86, wherein the fusion protein inhibits NK cells, M1 macrophages, T effector cells, and/or pro-inflammatory cytokines.

89. The method of any one of claims 84-88, wherein the fusion protein further decreases immunogenic response in the tissue.

90. The method of any one of claims 84-89, wherein the tolerogenic tissue environment restores durable response to defend a disease.

91. The method of any one of claims 84-90, wherein the durable immune response modulates inflammatory suppression in the tissues at the site of disease.

92. A method for selectively expanding ST2+ Treg cells in a subject comprising administering to the subject a bifunctional protein comprising an IL-2 variant associated with an IL-33 variant, wherein the IL-2 variant has amino acid substitution N88D and the IL-33 variant has amino acid substitution C116F, wherein the administration of the bifunctional protein results in eosinophil level at less than 5 fold, less than 4 fold, less than 3 fold or less than 2 fold as compared to the level before the treatment.

93. The method of claim 92, wherein the IL-2 variant has amino acid substitutions N88D and C125S and the IL-33 variant has amino acid substitutions selected from: (i) C97G and C116F; (ii) C97G, C116F and C148G; and (iii) C97G, C116F, C121S and C148G.94. The method of claim 92 or 93, wherein the administration of the bifunctional protein results in eosinophil level at less than 3 fold or less than 2 fold as compared to the level before the treatment.

95. A method of treating an autoimmune or inflammatory disease in a subject comprising administering to the subject the bifunctional protein or the fusion protein of any one of claims 1-70, or the composition of any one of claim 72 or 73.

96. The method of claim 95, wherein the disease is an autoimmune disease.

97. A population of Treg cells generated by contacting a T cell containing sample the bifunctional protein or the fusion protein of any one of claims 1-70, or the composition of any one of claim 72 or 73.

98. The population of Treg cells of claim 97, wherein the Treg cells are ST2+ Treg cells.

99. The population of ST2+ Treg cells of claim 97 or 98, wherein the T cell containing sample is a blood sample, a cell culture, or an iPSC-derived cell sample.
